SEQUENCE LISTING (626 sequences)

SEQ ID NO: 1 - protein, 10 aa, Artificial Sequence
  ligand of HLA-A*01 (A1)
YTDLLRLFEY

SEQ ID NO: 2 - protein, 12 aa, Artificial Sequence
  ligand of HLA-A*02 (A2)
SLLTSSKGQLQK

SEQ ID NO: 3 - protein, 10 aa, Artificial Sequence
  ligand of HLA-A*24 (A24)
EYPDRIMNTF

SEQ ID NO: 4 - protein, 11 aa, Artificial Sequence
  ligand of HLA-A*26 (A26)
ETFYVDGAANR

SEQ ID NO: 5 - protein, 12 aa, Artificial Sequence
  ligand of HLA-B*07 (B7)
VGGLKNWLVHRL

SEQ ID NO: 6 - protein, 9 aa, Artificial Sequence
  ligand of HLA-B*08 (B8)
MPHEKHYTL

SEQ ID NO: 7 - protein, 9 aa, Artificial Sequence
  ligand of HLA-B*35 (B35)
WASRELERF

SEQ ID NO: 8 - protein, 10 aa, Artificial Sequence
  ligand of HLA-B*40 (B40)
REIAQDFKTD

SEQ ID NO: 9 - protein, 9 aa, Artificial Sequence
  ligand of HLA-B*44 (B44)
DEYIYRHFF

SEQ ID NO: 10 - protein, 10 aa, Artificial Sequence
  ligand of HLA-B*51 (B51)
DTPLIPLTIF

SEQ ID NO: 11 - protein, 9 aa, Artificial Sequence
  ligand of HLA-Cw*03 (Cw3)
SAYGEPRKL

SEQ ID NO: 12 - protein, 10 aa, Artificial Sequence
  ligand of HLA-DPB1*0201
EIVDLMCHAT

SEQ ID NO: 13 - protein, 16 aa, Artificial Sequence
  ligand of HLA-DPB1*0401
IAFNSGMEPGVVAEKV

SEQ ID NO: 14 - protein, 15 aa, Artificial Sequence
  ligand of HLA-DQA1*0201
QPQQPQQSFPEQERP

SEQ ID NO: 15 - protein, 15 aa, Artificial Sequence
  ligand of HLA-DQA1*0301
HKLQDASAEVERLRR

SEQ ID NO: 16 - protein, 16 aa, Artificial Sequence
  ligand of HLA-DRB1*03 (DR3)
IPENLFLKSDGRVKYT

SEQ ID NO: 17 - protein, 18 aa, Artificial Sequence
  ligand of HLA-DRB1*0301 (DR17)
ISNQLTLDSNTKYFHKLN

SEQ ID NO: 18 - protein, 23 aa, Artificial Sequence
  ligand of HLA-DRB1*0701 (DR7)
KVDLTFSKQHALLCSDYQADYES

SEQ ID NO: 19 - protein, 15 aa, Artificial Sequence
  ligand of HLA-DRB1*11 (DR11)
KIFYVYMKRKYEAMT

SEQ ID NO: 20 - protein, 16 aa, Artificial Sequence
  ligand of HLA-DRB1*1501 (DR15)
DVGVYRAVTPQGRPDA

SEQ ID NO: 21 - protein, 8 aa, Artificial Sequence
  ligand of HLA-A*01
NTDSPLRY

SEQ ID NO: 22 - protein, 9 aa, Artificial Sequence
  ligand of HLA-A*01
PTDPKVVVY

SEQ ID NO: 23 - protein, 9 aa, Artificial Sequence
  ligand of HLA-A*01
VTEIDQDKY

SEQ ID NO: 24 - protein, 9 aa, Artificial Sequence
  ligand of HLA-A*01
AIDQLHLEY

SEQ ID NO: 25 - protein, 9 aa, Artificial Sequence
  ligand of HLA-A*01
SIDRTVMYY

SEQ ID NO: 26 - protein, 8 aa, Artificial Sequence
  ligand of HLA-A*01
LLDIRSEY

SEQ ID NO: 27 - protein, 11 aa, Artificial Sequence
  ligand of HLA-A*01
ETDLLDIRSEY

SEQ ID NO: 28 - protein, 11 aa, Artificial Sequence
  ligand of HLA-A*01
FLDASGAKLDY

SEQ ID NO: 29 - protein, 9 aa, Artificial Sequence
  ligand of HLA-A*01
FIDASRLVY

SEQ ID NO: 30 - protein, 9 aa, Artificial Sequence
  ligand of HLA-A*01
TVDQVKDLY

SEQ ID NO: 31 - protein, 10 aa, Artificial Sequence
  ligand of HLA-A*01
LLDQGQLNKY

SEQ ID NO: 32 - protein, 10 aa, Artificial Sequence
  ligand of HLA-A*01
YTETEPYHNY

SEQ ID NO: 33 - protein, 10 aa, Artificial Sequence
  ligand of HLA-A*01
STDKFKTDFY

SEQ ID NO: 34 - protein, 9 aa, Artificial Sequence
  ligand of HLA-A*01
IADMGHLKY

SEQ ID NO: 35 - protein, 9 aa, Artificial Sequence
  ligand of HLA-A*01
ATDFKFAMY

SEQ ID NO: 36 - protein, 12 aa, Artificial Sequence
  ligand of HLA-A*01
YTDYGGLIFNSY

SEQ ID NO: 37 - protein, 9 aa, Artificial Sequence
  ligand of HLA-A*01
VAVGRALYY

SEQ ID NO: 38 - protein, 9 aa, Artificial Sequence
  ligand of HLA-A*01
YLDDPDLKY

SEQ ID NO: 39 - protein, 9 aa, Artificial Sequence
  ligand of HLA-A*01
HLDLGILYY

SEQ ID NO: 40 - protein, 9 aa, Artificial Sequence
  ligand of HLA-A*01
VTDSIRDEY

SEQ ID NO: 41 - protein, 9 aa, Artificial Sequence
  ligand of HLA-A*01
LTDRELEEY

SEQ ID NO: 42 - protein, 9 aa, Artificial Sequence
  ligand of HLA-A*01
YTSDYFISY

SEQ ID NO: 43 - protein, 9 aa, Artificial Sequence
  ligand of HLA-A*01
ATDYHVRVY

SEQ ID NO: 44 - protein, 12 aa, Artificial Sequence
  ligand of HLA-A*01
VSDIVGPDGLVY

SEQ ID NO: 45 - protein, 9 aa, Artificial Sequence
  ligand of HLA-A*01
LEDVVIERY

SEQ ID NO: 46 - protein, 10 aa, Artificial Sequence
  ligand of HLA-A*01
ASDFITKMDY

SEQ ID NO: 47 - protein, 9 aa, Artificial Sequence
  ligand of HLA-A*01
YSEEECRQY

SEQ ID NO: 48 - protein, 9 aa, Artificial Sequence
  ligand of HLA-A*01
STDHIPILY

SEQ ID NO: 49 - protein, 10 aa, Artificial Sequence
  ligand of HLA-A*01
FTDVNSILRY

SEQ ID NO: 50 - protein, 10 aa, Artificial Sequence
  ligand of HLA-A*01
HTDDPLTWDY

SEQ ID NO: 51 - protein, 10 aa, Artificial Sequence
  ligand of HLA-A*01
TTEESLRNYY

SEQ ID NO: 52 - protein, 9 aa, Artificial Sequence
  ligand of HLA-A*01
STDHIPILY

SEQ ID NO: 53 - protein, 9 aa, Artificial Sequence
  ligand of HLA-A*01
LSDLGKLSY

SEQ ID NO: 54 - protein, 13 aa, Artificial Sequence
  ligand of HLA-A*01
QSEDGSHTIQIMY

SEQ ID NO: 55 - protein, 9 aa, Artificial Sequence
  ligand of HLA-A*01
YIDEQFERY

SEQ ID NO: 56 - protein, 9 aa, Artificial Sequence
  ligand of HLA-A*01
DSDGSFFLY

SEQ ID NO: 57 - protein, 9 aa, Artificial Sequence
  ligand of HLA-A*01
DSDGSFFLY

SEQ ID NO: 58 - protein, 9 aa, Artificial Sequence
  ligand of HLA-A*01
YTAVVPLVY

SEQ ID NO: 59 - protein, 9 aa, Artificial Sequence
  ligand of HLA-A*01
YTAVVPLVY

SEQ ID NO: 60 - protein, 9 aa, Artificial Sequence
  ligand of HLA-A*01
LTDRGVMSY

SEQ ID NO: 61 - protein, 9 aa, Artificial Sequence
  ligand of HLA-A*01
RADGNFLLY

SEQ ID NO: 62 - protein, 9 aa, Artificial Sequence
  ligand of HLA-A*01
GTDELRLLY

SEQ ID NO: 63 - protein, 9 aa, Artificial Sequence
  ligand of HLA-A*01
VLDPYLLKY

SEQ ID NO: 64 - protein, 10 aa, Artificial Sequence
  ligand of HLA-A*01
QVDPLSALKY

SEQ ID NO: 65 - protein, 9 aa, Artificial Sequence
  ligand of HLA-A*01
YSEKIVDMY

SEQ ID NO: 66 - protein, 9 aa, Artificial Sequence
  ligand of HLA-A*01
TSDQQRQLY

SEQ ID NO: 67 - protein, 9 aa, Artificial Sequence
  ligand of HLA-A*01
LAELAHREY

SEQ ID NO: 68 - protein, 9 aa, Artificial Sequence
  ligand of HLA-A*01
SSEQTFMYY

SEQ ID NO: 69 - protein, 9 aa, Artificial Sequence
  ligand of HLA-A*01
DTDHYFLRY

SEQ ID NO: 70 - protein, 9 aa, Artificial Sequence
  ligand of HLA-A*01
ELEDSTLRY

SEQ ID NO: 71 - protein, 9 aa, Artificial Sequence
  ligand of HLA-A*01
YVDPQFLTY

SEQ ID NO: 72 - protein, 9 aa, Artificial Sequence
  ligand of HLA-A*01
IADMGHLKY

SEQ ID NO: 73 - protein, 9 aa, Artificial Sequence
  ligand of HLA-A*01
YTDPEVFKY

SEQ ID NO: 74 - protein, 11 aa, Artificial Sequence
  ligand of HLA-A*01
STDVSDLLHQY

SEQ ID NO: 75 - protein, 10 aa, Artificial Sequence
  ligand of HLA-A*01
QAQADSLTVY

SEQ ID NO: 76 - protein, 10 aa, Artificial Sequence
  ligand of HLA-A*01
STDKAEYTFY

SEQ ID NO: 77 - protein, 9 aa, Artificial Sequence
  ligand of HLA-A*01
MIEPRTLQY

SEQ ID NO: 78 - protein, 9 aa, Artificial Sequence
  ligand of HLA-A*01
MIEPRTLQY

SEQ ID NO: 79 - protein, 9 aa, Artificial Sequence
  ligand of HLA-A*01
TTEVHPELY

SEQ ID NO: 80 - protein, 9 aa, Artificial Sequence
  ligand of HLA-A*01
TSPSQSLFY

SEQ ID NO: 81 - protein, 9 aa, Artificial Sequence
  ligand of HLA-A*01
TTEQHGARY

SEQ ID NO: 82 - protein, 11 aa, Artificial Sequence
  ligand of HLA-A*01
HTQGPVDGSLY

SEQ ID NO: 83 - protein, 10 aa, Artificial Sequence
  ligand of HLA-A*01
STDPSVLGKY

SEQ ID NO: 84 - protein, 10 aa, Artificial Sequence
  ligand of HLA-A*01
YTDKIDRYTR

SEQ ID NO: 85 - protein, 10 aa, Artificial Sequence
  ligand of HLA-A*01
ITDSAGHILY

SEQ ID NO: 86 - protein, 9 aa, Artificial Sequence
  ligand of HLA-A*01
VTEIFRQAF

SEQ ID NO: 87 - protein, 11 aa, Artificial Sequence
  ligand of HLA-A*01
HTQGPVDGSLY

SEQ ID NO: 88 - protein, 11 aa, Artificial Sequence
  ligand of HLA-A*01
QLEDGRTLSDY

SEQ ID NO: 89 - protein, 9 aa, Artificial Sequence
  ligand of HLA-A*01
IVDCLTEMY

SEQ ID NO: 90 - protein, 11 aa, Artificial Sequence
  ligand of HLA-A*01
ALRPSTSRSLY

SEQ ID NO: 91 - protein, 9 aa, Artificial Sequence
  ligand of HLA-A*01
ISNQEPLKL

SEQ ID NO: 92 - protein, 11 aa, Artificial Sequence
  ligand of HLA-A*01
YSEHPTFTSQY

SEQ ID NO: 93 - protein, 9 aa, Artificial Sequence
  ligand of HLA-A*01
GSEELRSLY

SEQ ID NO: 94 - protein, 9 aa, Artificial Sequence
  ligand of HLA-A*01
YAETKHFLY

SEQ ID NO: 95 - protein, 9 aa, Artificial Sequence
  ligand of HLA-A*01
CTELKLSDY

SEQ ID NO: 96 - protein, 9 aa, Artificial Sequence
  ligand of HLA-A*01
VSDGGPNLY

SEQ ID NO: 97 - protein, 9 aa, Artificial Sequence
  ligand of HLA-A*01
VTEPGTAQY

SEQ ID NO: 98 - protein, 10 aa, Artificial Sequence
  ligand of HLA-A*01
VYDFFVWLHY

SEQ ID NO: 99 - protein, 9 aa, Artificial Sequence
  ligand of HLA-A*01
EADPTGHSY

SEQ ID NO: 100 - protein, 9 aa, Artificial Sequence
  ligand of HLA-A*01
EVDPIGHLY

SEQ ID NO: 101 - protein, 9 aa, Artificial Sequence
  ligand of HLA-A*01
IVDCLTEMY

SEQ ID NO: 102 - protein, 9 aa, Artificial Sequence
  ligand of HLA-A*01
ILDSSEEDK

SEQ ID NO: 103 - protein, 9 aa, Artificial Sequence
  ligand of HLA-A*01
KCDICTDEY

SEQ ID NO: 104 - protein, 11 aa, Artificial Sequence
  ligand of HLA-A*01
SSDYVIPIGTY

SEQ ID NO: 105 - protein, 10 aa, Artificial Sequence
  ligand of HLA-A*01
DSDPDSFQDY

SEQ ID NO: 106 - protein, 10 aa, Artificial Sequence
  ligand of HLA-A*03
KLFDHAVSKF

SEQ ID NO: 107 - protein, 9 aa, Artificial Sequence
  ligand of HLA-A*03
SLWDRLIFH

SEQ ID NO: 108 - protein, 12 aa, Artificial Sequence
  ligand of HLA-A*03
SLLTSSKGQLQK

SEQ ID NO: 109 - protein, 9 aa, Artificial Sequence
  ligand of HLA-A*03
TSALPIIQK

SEQ ID NO: 110 - protein, 9 aa, Artificial Sequence
  ligand of HLA-A*03
SLFSRLFGK

SEQ ID NO: 111 - protein, 9 aa, Artificial Sequence
  ligand of HLA-A*03
KLYEMILKR

SEQ ID NO: 112 - protein, 10 aa, Artificial Sequence
  ligand of HLA-A*03
GTYAPAEVPK

SEQ ID NO: 113 - protein, 9 aa, Artificial Sequence
  ligand of HLA-A*03
RLLEMILNK

SEQ ID NO: 114 - protein, 9 aa, Artificial Sequence
  ligand of HLA-A*03
KLFDKLLEY

SEQ ID NO: 115 - protein, 10 aa, Artificial Sequence
  ligand of HLA-A*03
RIIEETLALK

SEQ ID NO: 116 - protein, 9 aa, Artificial Sequence
  ligand of HLA-A*03
IVALILSTK

SEQ ID NO: 117 - protein, 9 aa, Artificial Sequence
  ligand of HLA-A*03
KIKSFEVVF

SEQ ID NO: 118 - protein, 9 aa, Artificial Sequence
  ligand of HLA-A*03
KIADRFLLY

SEQ ID NO: 119 - protein, 9 aa, Artificial Sequence
  ligand of HLA-A*03
GLYEFFRAK

SEQ ID NO: 120 - protein, 8 aa, Artificial Sequence
  ligand of HLA-A*03
FVQMMTAK

SEQ ID NO: 121 - protein, 9 aa, Artificial Sequence
  ligand of HLA-A*03
KVMQQNLVY

SEQ ID NO: 122 - protein, 9 aa, Artificial Sequence
  ligand of HLA-A*03
KVFQEPLFY

SEQ ID NO: 123 - protein, 9 aa, Artificial Sequence
  ligand of HLA-A*03
GLMGFIVYK

SEQ ID NO: 124 - protein, 9 aa, Artificial Sequence
  ligand of HLA-A*03
QISIPFLLK

SEQ ID NO: 125 - protein, 9 aa, Artificial Sequence
  ligand of HLA-A*03
KLADFGLAR

SEQ ID NO: 126 - protein, 9 aa, Artificial Sequence
  ligand of HLA-A*03
RMFDMGFEY

SEQ ID NO: 127 - protein, 9 aa, Artificial Sequence
  ligand of HLA-A*03
KVYENYPTY

SEQ ID NO: 128 - protein, 9 aa, Artificial Sequence
  ligand of HLA-A*03
TVAVPLVGK

SEQ ID NO: 129 - protein, 9 aa, Artificial Sequence
  ligand of HLA-A*03
ESYEALPQH

SEQ ID NO: 130 - protein, 9 aa, Artificial Sequence
  ligand of HLA-A*03
ATDPNILGR

SEQ ID NO: 131 - protein, 9 aa, Artificial Sequence
  ligand of HLA-A*03
TLADLLALR

SEQ ID NO: 132 - protein, 9 aa, Artificial Sequence
  ligand of HLA-A*03
SLMHSFILK

SEQ ID NO: 133 - protein, 9 aa, Artificial Sequence
  ligand of HLA-A*03
RVHAYIISY

SEQ ID NO: 134 - protein, 9 aa, Artificial Sequence
  ligand of HLA-A*03
SVLSPLLNK

SEQ ID NO: 135 - protein, 9 aa, Artificial Sequence
  ligand of HLA-A*03
KIKSFEVVF

SEQ ID NO: 136 - protein, 9 aa, Artificial Sequence
  ligand of HLA-A*03
TLADILLYY

SEQ ID NO: 137 - protein, 12 aa, Artificial Sequence
  ligand of HLA-A*03
RVFSGLVSTGLK

SEQ ID NO: 138 - protein, 9 aa, Artificial Sequence
  ligand of HLA-A*03
AVAIKAMAK

SEQ ID NO: 139 - protein, 9 aa, Artificial Sequence
  ligand of HLA-A*03
ILENIQRNK

SEQ ID NO: 140 - protein, 9 aa, Artificial Sequence
  ligand of HLA-A*03
IVAGSLITK

SEQ ID NO: 141 - protein, 9 aa, Artificial Sequence
  ligand of HLA-A*03
IIYKGGTSR

SEQ ID NO: 142 - protein, 9 aa, Artificial Sequence
  ligand of HLA-A*03
QIASFILLR

SEQ ID NO: 143 - protein, 10 aa, Artificial Sequence
  ligand of HLA-A*03
ATGSWDSFLK

SEQ ID NO: 144 - protein, 9 aa, Artificial Sequence
  ligand of HLA-A*03
SVYVYKVLK

SEQ ID NO: 145 - protein, 10 aa, Artificial Sequence
  ligand of HLA-A*03
KVCNPIITKL

SEQ ID NO: 146 - protein, 9 aa, Artificial Sequence
  ligand of HLA-A*03
KLFIGGLSF

SEQ ID NO: 147 - protein, 9 aa, Artificial Sequence
  ligand of HLA-A*03
RLFVGSPIK

SEQ ID NO: 148 - protein, 9 aa, Artificial Sequence
  ligand of HLA-A*03
TLIDIMTRF

SEQ ID NO: 149 - protein, 9 aa, Artificial Sequence
  ligand of HLA-A*03
NVIRDAVTY

SEQ ID NO: 150 - protein, 11 aa, Artificial Sequence
  ligand of HLA-A*03
ALLDGSNVVFK

SEQ ID NO: 151 - protein, 12 aa, Artificial Sequence
  ligand of HLA-A*03
GLFEVGAGWIGK

SEQ ID NO: 152 - protein, 11 aa, Artificial Sequence
  ligand of HLA-A*03
RVQEAVESMVK

SEQ ID NO: 153 - protein, 10 aa, Artificial Sequence
  ligand of HLA-A*03
VVNKVPLTGK

SEQ ID NO: 154 - protein, 10 aa, Artificial Sequence
  ligand of HLA-A*03
ALPSRILLWK

SEQ ID NO: 155 - protein, 9 aa, Artificial Sequence
  ligand of HLA-A*03
RLLQKVLAY

SEQ ID NO: 156 - protein, 9 aa, Artificial Sequence
  ligand of HLA-A*03
SLAEILLKK

SEQ ID NO: 157 - protein, 10 aa, Artificial Sequence
  ligand of HLA-A*03
AVNAHSNILK

SEQ ID NO: 158 - protein, 9 aa, Artificial Sequence
  ligand of HLA-A*03
ATFPDTLTY

SEQ ID NO: 159 - protein, 9 aa, Artificial Sequence
  ligand of HLA-A*03
HLEDIVRQK

SEQ ID NO: 160 - protein, 10 aa, Artificial Sequence
  ligand of HLA-A*03
AVFPKPFVEK

SEQ ID NO: 161 - protein, 9 aa, Artificial Sequence
  ligand of HLA-A*03
QTYVGITEK

SEQ ID NO: 162 - protein, 9 aa, Artificial Sequence
  ligand of HLA-A*03
AIIDHIFAS

SEQ ID NO: 163 - protein, 11 aa, Artificial Sequence
  ligand of HLA-A*03
TVMELVKIIYK

SEQ ID NO: 164 - protein, 9 aa, Artificial Sequence
  ligand of HLA-A*03
KVYNIQIRY

SEQ ID NO: 165 - protein, 9 aa, Artificial Sequence
  ligand of HLA-A*03
SVYVLVRQK

SEQ ID NO: 166 - protein, 9 aa, Artificial Sequence
  ligand of HLA-A*03
FPNSPKWTS

SEQ ID NO: 167 - protein, 12 aa, Artificial Sequence
  ligand of HLA-A*03
SLFPNSPKWTSK

SEQ ID NO: 168 - protein, 9 aa, Artificial Sequence
  ligand of HLA-A*03
SIFKQPVTK

SEQ ID NO: 169 - protein, 10 aa, Artificial Sequence
  ligand of HLA-A*03
VLFGKALNPK

SEQ ID NO: 170 - protein, 9 aa, Artificial Sequence
  ligand of HLA-A*03
RVFPWFSVK

SEQ ID NO: 171 - protein, 9 aa, Artificial Sequence
  ligand of HLA-A*03
KTYGEIFEK

SEQ ID NO: 172 - protein, 9 aa, Artificial Sequence
  ligand of HLA-A*03
GVLAWVKEK

SEQ ID NO: 173 - protein, 9 aa, Artificial Sequence
  ligand of HLA-A*03
GVADKILKK

SEQ ID NO: 174 - protein, 9 aa, Artificial Sequence
  ligand of HLA-A*03
ALLAVGATK

SEQ ID NO: 175 - protein, 10 aa, Artificial Sequence
  ligand of HLA-A*03
KLLNYAPLEK

SEQ ID NO: 176 - protein, 9 aa, Artificial Sequence
  ligand of HLA-A*03
AMLDTVVFK

SEQ ID NO: 177 - protein, 10 aa, Artificial Sequence
  ligand of HLA-A*03
GSYNKVFLAK

SEQ ID NO: 178 - protein, 9 aa, Artificial Sequence
  ligand of HLA-A*03
RILFFNTPK

SEQ ID NO: 179 - protein, 11 aa, Artificial Sequence
  ligand of HLA-A*03
AALVASGVALY

SEQ ID NO: 180 - protein, 10 aa, Artificial Sequence
  ligand of HLA-A*03
SIFDGRVVAK

SEQ ID NO: 181 - protein, 10 aa, Artificial Sequence
  ligand of HLA-A*03
KLPNFGFVVF

SEQ ID NO: 182 - protein, 10 aa, Artificial Sequence
  ligand of HLA-A*03
GLASFKSFLK

SEQ ID NO: 183 - protein, 9 aa, Artificial Sequence
  ligand of HLA-A*03
LTGPVMPVR

SEQ ID NO: 184 - protein, 9 aa, Artificial Sequence
  ligand of HLA-A*03
QLYWSHPRK

SEQ ID NO: 185 - protein, 10 aa, Artificial Sequence
  ligand of HLA-A*03
LVFPSEIVGK

SEQ ID NO: 186 - protein, 11 aa, Artificial Sequence
  ligand of HLA-A*03
AVIVLVENFYK

SEQ ID NO: 187 - protein, 10 aa, Artificial Sequence
  ligand of HLA-A*03
RLYGPSSVSF

SEQ ID NO: 188 - protein, 9 aa, Artificial Sequence
  ligand of HLA-A*03
VLYDRVLKY

SEQ ID NO: 189 - protein, 9 aa, Artificial Sequence
  ligand of HLA-A*03
GTLPHPLQR

SEQ ID NO: 190 - protein, 9 aa, Artificial Sequence
  ligand of HLA-A*03
TSPSQSLFY

SEQ ID NO: 191 - protein, 9 aa, Artificial Sequence
  ligand of HLA-A*03
YLADFLLTK

SEQ ID NO: 192 - protein, 9 aa, Artificial Sequence
  ligand of HLA-A*03
RQIPYTMMK

SEQ ID NO: 193 - protein, 9 aa, Artificial Sequence
  ligand of HLA-A*03
RVYIGRLSY

SEQ ID NO: 194 - protein, 9 aa, Artificial Sequence
  ligand of HLA-A*03
SVYDSVLQK

SEQ ID NO: 195 - protein, 9 aa, Artificial Sequence
  ligand of HLA-A*03
GLTDVILYH

SEQ ID NO: 196 - protein, 10 aa, Artificial Sequence
  ligand of HLA-A*03
SIGERDLIFH

SEQ ID NO: 197 - protein, 9 aa, Artificial Sequence
  ligand of HLA-A*03
SITSVFITK

SEQ ID NO: 198 - protein, 9 aa, Artificial Sequence
  ligand of HLA-A*03
GTMTGMLYK

SEQ ID NO: 199 - protein, 9 aa, Artificial Sequence
  ligand of HLA-A*03
RIHFPLATY

SEQ ID NO: 200 - protein, 10 aa, Artificial Sequence
  ligand of HLA-A*03
QIFVKTLTGK

SEQ ID NO: 201 - protein, 9 aa, Artificial Sequence
  ligand of HLA-A*03
SQNFPGSQK

SEQ ID NO: 202 - protein, 9 aa, Artificial Sequence
  ligand of HLA-A*03
ALRDVRQQY

SEQ ID NO: 203 - protein, 11 aa, Artificial Sequence
  ligand of HLA-A*03
ALRPSTSRSLY

SEQ ID NO: 204 - protein, 11 aa, Artificial Sequence
  ligand of HLA-A*03
SLYASSPGGVY

SEQ ID NO: 205 - protein, 14 aa, Artificial Sequence
  ligand of HLA-A*03
SLYASSPGGVYATR

SEQ ID NO: 206 - protein, 9 aa, Artificial Sequence
  ligand of HLA-A*03
RTGPPMGSR

SEQ ID NO: 207 - protein, 11 aa, Artificial Sequence
  ligand of HLA-A*03
HSATGFKQSSK

SEQ ID NO: 208 - protein, 9 aa, Artificial Sequence
  ligand of HLA-A*03
KQSSKALQR

SEQ ID NO: 209 - protein, 9 aa, Artificial Sequence
  ligand of HLA-A*03
RLRAEAQVK

SEQ ID NO: 210 - protein, 9 aa, Artificial Sequence
  ligand of HLA-A*03
NTYASPRKF

SEQ ID NO: 211 - protein, 9 aa, Artificial Sequence
  ligand of HLA-A*03
RVCEKMALY

SEQ ID NO: 212 - protein, 10 aa, Artificial Sequence
  ligand of HLA-A*03
QVPLRPMTYK

SEQ ID NO: 213 - protein, 10 aa, Artificial Sequence
  ligand of HLA-A*03
TVYYGVPVWK

SEQ ID NO: 214 - protein, 11 aa, Artificial Sequence
  ligand of HLA-A*03
RLRDLLLIVTR

SEQ ID NO: 215 - protein, 9 aa, Artificial Sequence
  ligand of HLA-A*03
RLRPGGKKK

SEQ ID NO: 216 - protein, 9 aa, Artificial Sequence
  ligand of HLA-A*03
KIRLRPGGK

SEQ ID NO: 217 - protein, 9 aa, Artificial Sequence
  ligand of HLA-A*03
ILRGSVAHK

SEQ ID NO: 218 - protein, 9 aa, Artificial Sequence
  ligand of HLA-A*03
SLFRAVITK

SEQ ID NO: 219 - protein, 9 aa, Artificial Sequence
  ligand of HLA-A*03
RLEDVFAGK

SEQ ID NO: 220 - protein, 10 aa, Artificial Sequence
  ligand of HLA-A*03
ASGPGGGAPR

SEQ ID NO: 221 - protein, 9 aa, Artificial Sequence
  ligand of HLA-A*03
KLIETYFSK

SEQ ID NO: 222 - protein, 9 aa, Artificial Sequence
  ligand of HLA-A*03
LIYRRRLMK

SEQ ID NO: 223 - protein, 9 aa, Artificial Sequence
  ligand of HLA-A*03
ALNFPGSQK

SEQ ID NO: 224 - protein, 9 aa, Artificial Sequence
  ligand of HLA-A*03
QVHPQKVTK

SEQ ID NO: 225 - protein, 9 aa, Artificial Sequence
  ligand of HLA-A*03
RIETRSARH

SEQ ID NO: 226 - protein, 9 aa, Artificial Sequence
  ligand of HLA-A*03
LLGPGRPYR

SEQ ID NO: 227 - protein, 9 aa, Artificial Sequence
  ligand of HLA-A*24
RYFDEPVEL

SEQ ID NO: 228 - protein, 9 aa, Artificial Sequence
  ligand of HLA-A*24
VYISEHEHF

SEQ ID NO: 229 - protein, 9 aa, Artificial Sequence
  ligand of HLA-A*24
IYTSSVNRL

SEQ ID NO: 230 - protein, 9 aa, Artificial Sequence
  ligand of HLA-A*24
KYDEIFYNL

SEQ ID NO: 231 - protein, 11 aa, Artificial Sequence
  ligand of HLA-A*24
KYITQGQLLQF

SEQ ID NO: 232 - protein, 9 aa, Artificial Sequence
  ligand of HLA-A*24
EYIVKKIPV

SEQ ID NO: 233 - protein, 9 aa, Artificial Sequence
  ligand of HLA-A*24
LYILSSHDI

SEQ ID NO: 234 - protein, 9 aa, Artificial Sequence
  ligand of HLA-A*24
AYQHLFYLL

SEQ ID NO: 235 - protein, 9 aa, Artificial Sequence
  ligand of HLA-A*24
KYFLKPEVL

SEQ ID NO: 236 - protein, 9 aa, Artificial Sequence
  ligand of HLA-A*24
KYPSPFFVF

SEQ ID NO: 237 - protein, 9 aa, Artificial Sequence
  ligand of HLA-A*24
RYSSMAASF

SEQ ID NO: 238 - protein, 9 aa, Artificial Sequence
  ligand of HLA-A*24
KYLSVQGQL

SEQ ID NO: 239 - protein, 9 aa, Artificial Sequence
  ligand of HLA-A*24
TYGEIFEKF

SEQ ID NO: 240 - protein, 9 aa, Artificial Sequence
  ligand of HLA-A*24
YYMIGEQKF

SEQ ID NO: 241 - protein, 9 aa, Artificial Sequence
  ligand of HLA-A*24
YYEEQHPEL

SEQ ID NO: 242 - protein, 9 aa, Artificial Sequence
  ligand of HLA-A*24
SYIEHIFEI

SEQ ID NO: 243 - protein, 9 aa, Artificial Sequence
  ligand of HLA-A*24
TYTDRVFFL

SEQ ID NO: 244 - protein, 9 aa, Artificial Sequence
  ligand of HLA-A*24
VYIKHPVSL

SEQ ID NO: 245 - protein, 9 aa, Artificial Sequence
  ligand of HLA-A*24
KYPENFFLL

SEQ ID NO: 246 - protein, 9 aa, Artificial Sequence
  ligand of HLA-A*24
KYPENFFLL

SEQ ID NO: 247 - protein, 9 aa, Artificial Sequence
  ligand of HLA-A*24
KFIDTTSKF

SEQ ID NO: 248 - protein, 9 aa, Artificial Sequence
  ligand of HLA-A*24
LYPQFMFHL

SEQ ID NO: 249 - protein, 9 aa, Artificial Sequence
  ligand of HLA-A*24
RYIPTAAAF

SEQ ID NO: 250 - protein, 9 aa, Artificial Sequence
  ligand of HLA-A*24
VYPDGIRHI

SEQ ID NO: 251 - protein, 9 aa, Artificial Sequence
  ligand of HLA-A*24
KYILLMDII

SEQ ID NO: 252 - protein, 9 aa, Artificial Sequence
  ligand of HLA-A*24
AYVHMVTHF

SEQ ID NO: 253 - protein, 10 aa, Artificial Sequence
  ligand of HLA-A*24
KFIDPIYQVW

SEQ ID NO: 254 - protein, 9 aa, Artificial Sequence
  ligand of HLA-A*24
KYQKGFSLW

SEQ ID NO: 255 - protein, 9 aa, Artificial Sequence
  ligand of HLA-A*24
NYIDKVRFL

SEQ ID NO: 256 - protein, 9 aa, Artificial Sequence
  ligand of HLA-A*24
EYCLKFTKL

SEQ ID NO: 257 - protein, 9 aa, Artificial Sequence
  ligand of HLA-A*24
SYLDSGIHF

SEQ ID NO: 258 - protein, 10 aa, Artificial Sequence
  ligand of HLA-A*24
QYSWFVNGTF

SEQ ID NO: 259 - protein, 8 aa, Artificial Sequence
  ligand of HLA-A*24
RYSIFFDY

SEQ ID NO: 260 - protein, 9 aa, Artificial Sequence
  ligand of HLA-A*24
TYGPVFMCL

SEQ ID NO: 261 - protein, 10 aa, Artificial Sequence
  ligand of HLA-A*24
DYCNVLNKEF

SEQ ID NO: 262 - protein, 9 aa, Artificial Sequence
  ligand of HLA-A*24
RWGLLLALL

SEQ ID NO: 263 - protein, 9 aa, Artificial Sequence
  ligand of HLA-A*24
RYLKDQQLL

SEQ ID NO: 264 - protein, 9 aa, Artificial Sequence
  ligand of HLA-A*24
KYKLKHIVW

SEQ ID NO: 265 - protein, 10 aa, Artificial Sequence
  ligand of HLA-A*24
LFCASDAKAY

SEQ ID NO: 266 - protein, 10 aa, Artificial Sequence
  ligand of HLA-A*24
QYNKPLCDLL

SEQ ID NO: 267 - protein, 9 aa, Artificial Sequence
  ligand of HLA-A*24
EYRHYCYSL

SEQ ID NO: 268 - protein, 10 aa, Artificial Sequence
  ligand of HLA-A*24
AYAQKIFKIL

SEQ ID NO: 269 - protein, 9 aa, Artificial Sequence
  ligand of HLA-A*24
NYKHCFPEI

SEQ ID NO: 270 - protein, 9 aa, Artificial Sequence
  ligand of HLA-A*24
IMPKAGLLI

SEQ ID NO: 271 - protein, 9 aa, Artificial Sequence
  ligand of HLA-A*24
KTPFVSPLL

SEQ ID NO: 272 - protein, 9 aa, Artificial Sequence
  ligand of HLA-A*24
AYGLDFYIL

SEQ ID NO: 273 - protein, 9 aa, Artificial Sequence
  ligand of HLA-A*24
LYVDSLFFL

SEQ ID NO: 274 - protein, 9 aa, Artificial Sequence
  ligand of HLA-A*24
EYRGFTQDF

SEQ ID NO: 275 - protein, 9 aa, Artificial Sequence
  ligand of HLA-A*24
AFLPWHRLF

SEQ ID NO: 276 - protein, 9 aa, Artificial Sequence
  ligand of HLA-A*24
AFLPWHRLF

SEQ ID NO: 277 - protein, 9 aa, Artificial Sequence
  ligand of HLA-A*26
EVIPMFSAL

SEQ ID NO: 278 - protein, 9 aa, Artificial Sequence
  ligand of HLA-B*07
APAPPPKPM

SEQ ID NO: 279 - protein, 10 aa, Artificial Sequence
  ligand of HLA-B*07
PSRDSLPLPV

SEQ ID NO: 280 - protein, 10 aa, Artificial Sequence
  ligand of HLA-B*07
VPNQKRLTLL

SEQ ID NO: 281 - protein, 9 aa, Artificial Sequence
  ligand of HLA-B*07
IARNLTQQL

SEQ ID NO: 282 - protein, 9 aa, Artificial Sequence
  ligand of HLA-B*07
RPELVRPAL

SEQ ID NO: 283 - protein, 9 aa, Artificial Sequence
  ligand of HLA-B*07
KPRSPVVEL

SEQ ID NO: 284 - protein, 9 aa, Artificial Sequence
  ligand of HLA-B*07
WPLWRLVSL

SEQ ID NO: 285 - protein, 10 aa, Artificial Sequence
  ligand of HLA-B*07
SPRAAEPVQL

SEQ ID NO: 286 - protein, 9 aa, Artificial Sequence
  ligand of HLA-B*07
RPKSNIVLL

SEQ ID NO: 287 - protein, 9 aa, Artificial Sequence
  ligand of HLA-B*07
FVIETARQL

SEQ ID NO: 288 - protein, 9 aa, Artificial Sequence
  ligand of HLA-B*07
APSPRPLSL

SEQ ID NO: 289 - protein, 9 aa, Artificial Sequence
  ligand of HLA-B*07
IVPANFPSL

SEQ ID NO: 290 - protein, 9 aa, Artificial Sequence
  ligand of HLA-B*07
MPRGVVVTL

SEQ ID NO: 291 - protein, 9 aa, Artificial Sequence
  ligand of HLA-B*07
LPKPPGRGV

SEQ ID NO: 292 - protein, 8 aa, Artificial Sequence
  ligand of HLA-B*07
APLLRWVL

SEQ ID NO: 293 - protein, 11 aa, Artificial Sequence
  ligand of HLA-B*07
VPDSSGPERIL

SEQ ID NO: 294 - protein, 9 aa, Artificial Sequence
  ligand of HLA-B*07
APKRPPSAF

SEQ ID NO: 295 - protein, 11 aa, Artificial Sequence
  ligand of HLA-B*07
AASKERSGVSL

SEQ ID NO: 296 - protein, 9 aa, Artificial Sequence
  ligand of HLA-B*07
APRTLVLLL

SEQ ID NO: 297 - protein, 9 aa, Artificial Sequence
  ligand of HLA-B*07
APRTVLLLL

SEQ ID NO: 298 - protein, 9 aa, Artificial Sequence
  ligand of HLA-B*07
LVMAPRTVL

SEQ ID NO: 299 - protein, 9 aa, Artificial Sequence
  ligand of HLA-B*07
APRALLLLL

SEQ ID NO: 300 - protein, 9 aa, Artificial Sequence
  ligand of HLA-B*07
RVMAPRALL

SEQ ID NO: 301 - protein, 9 aa, Artificial Sequence
  ligand of HLA-B*07
APRALLLLL

SEQ ID NO: 302 - protein, 9 aa, Artificial Sequence
  ligand of HLA-B*07
RVMAPRALL

SEQ ID NO: 303 - protein, 11 aa, Artificial Sequence
  ligand of HLA-B*07
APRTVALTALL

SEQ ID NO: 304 - protein, 9 aa, Artificial Sequence
  ligand of HLA-B*07
APRTVALTA

SEQ ID NO: 305 - protein, 10 aa, Artificial Sequence
  ligand of HLA-B*07
APRTVALTAL

SEQ ID NO: 306 - protein, 10 aa, Artificial Sequence
  ligand of HLA-B*07
SPHLANYFYF

SEQ ID NO: 307 - protein, 9 aa, Artificial Sequence
  ligand of HLA-B*07
RPTLWAAAL

SEQ ID NO: 308 - protein, 9 aa, Artificial Sequence
  ligand of HLA-B*07
SPRLPVGGF

SEQ ID NO: 309 - protein, 11 aa, Artificial Sequence
  ligand of HLA-B*07
RPSTPKSDSEL

SEQ ID NO: 310 - protein, 14 aa, Artificial Sequence
  ligand of HLA-B*07
GPPGTGKTDVAVQI

SEQ ID NO: 311 - protein, 9 aa, Artificial Sequence
  ligand of HLA-B*07
RPAKSMDSL

SEQ ID NO: 312 - protein, 9 aa, Artificial Sequence
  ligand of HLA-B*07
KPNANRIAL

SEQ ID NO: 313 - protein, 9 aa, Artificial Sequence
  ligand of HLA-B*07
TPMGPGRTV

SEQ ID NO: 314 - protein, 9 aa, Artificial Sequence
  ligand of HLA-B*07
RPVSPGKDI

SEQ ID NO: 315 - protein, 9 aa, Artificial Sequence
  ligand of HLA-B*07
GPRSASLLS

SEQ ID NO: 316 - protein, 11 aa, Artificial Sequence
  ligand of HLA-B*07
SPSVDKARAEL

SEQ ID NO: 317 - protein, 9 aa, Artificial Sequence
  ligand of HLA-B*07
RPASAGAML

SEQ ID NO: 318 - protein, 9 aa, Artificial Sequence
  ligand of HLA-B*07
LVLMVLYLI

SEQ ID NO: 319 - protein, 12 aa, Artificial Sequence
  ligand of HLA-B*07
APRIITGPAPVL

SEQ ID NO: 320 - protein, 10 aa, Artificial Sequence
  ligand of HLA-B*07
IPRAALLPLL

SEQ ID NO: 321 - protein, 10 aa, Artificial Sequence
  ligand of HLA-B*07
KPRDVSSVEL

SEQ ID NO: 322 - protein, 10 aa, Artificial Sequence
  ligand of HLA-B*07
SPRENILVSL

SEQ ID NO: 323 - protein, 9 aa, Artificial Sequence
  ligand of HLA-B*07
APARLFALL

SEQ ID NO: 324 - protein, 9 aa, Artificial Sequence
  ligand of HLA-B*07
SPRYIFTML

SEQ ID NO: 325 - protein, 9 aa, Artificial Sequence
  ligand of HLA-B*07
RPSGPGPEL

SEQ ID NO: 326 - protein, 9 aa, Artificial Sequence
  ligand of HLA-B*07
HASDRIIAL

SEQ ID NO: 327 - protein, 11 aa, Artificial Sequence
  ligand of HLA-B*07
VVYVGGILTIL

SEQ ID NO: 328 - protein, 9 aa, Artificial Sequence
  ligand of HLA-B*07
VPAPAGPIV

SEQ ID NO: 329 - protein, 9 aa, Artificial Sequence
  ligand of HLA-B*07
CRVLCCYVL

SEQ ID NO: 330 - protein, 10 aa, Artificial Sequence
  ligand of HLA-B*07
TPRVTGGGAM

SEQ ID NO: 331 - protein, 9 aa, Artificial Sequence
  ligand of HLA-B*07
GPRLGVRAT

SEQ ID NO: 332 - protein, 9 aa, Artificial Sequence
  ligand of HLA-B*07
FPVRPQVPL

SEQ ID NO: 333 - protein, 10 aa, Artificial Sequence
  ligand of HLA-B*07
TPGPGIRYPL

SEQ ID NO: 334 - protein, 9 aa, Artificial Sequence
  ligand of HLA-B*07
RPMTYKAAL

SEQ ID NO: 335 - protein, 10 aa, Artificial Sequence
  ligand of HLA-B*07
RPNNNTRKSI

SEQ ID NO: 336 - protein, 9 aa, Artificial Sequence
  ligand of HLA-B*07
IPRRIRQGL

SEQ ID NO: 337 - protein, 9 aa, Artificial Sequence
  ligand of HLA-B*07
GPGHKARVL

SEQ ID NO: 338 - protein, 9 aa, Artificial Sequence
  ligand of HLA-B*07
SPRTLNAWV

SEQ ID NO: 339 - protein, 9 aa, Artificial Sequence
  ligand of HLA-B*07
ATPQDLNTM

SEQ ID NO: 340 - protein, 9 aa, Artificial Sequence
  ligand of HLA-B*07
RVRFFFPSL

SEQ ID NO: 341 - protein, 9 aa, Artificial Sequence
  ligand of HLA-B*07
MPNDPNRNV

SEQ ID NO: 342 - protein, 10 aa, Artificial Sequence
  ligand of HLA-B*07
SPSSNRIRNT

SEQ ID NO: 343 - protein, 9 aa, Artificial Sequence
  ligand of HLA-B*07
NPKASLLSL

SEQ ID NO: 344 - protein, 9 aa, Artificial Sequence
  ligand of HLA-B*08
VPKLKVCAL

SEQ ID NO: 345 - protein, 9 aa, Artificial Sequence
  ligand of HLA-B*08
YLKVKGNVF

SEQ ID NO: 346 - protein, 8 aa, Artificial Sequence
  ligand of HLA-B*08
FPRLKSKL

SEQ ID NO: 347 - protein, 9 aa, Artificial Sequence
  ligand of HLA-B*08
VGLIRNLAL

SEQ ID NO: 348 - protein, 8 aa, Artificial Sequence
  ligand of HLA-B*08
SLKEKVLL

SEQ ID NO: 349 - protein, 9 aa, Artificial Sequence
  ligand of HLA-B*08
MPLLRQEEL

SEQ ID NO: 350 - protein, 8 aa, Artificial Sequence
  ligand of HLA-B*08
LPKVKLAL

SEQ ID NO: 351 - protein, 8 aa, Artificial Sequence
  ligand of HLA-B*08
ELLIRKLP

SEQ ID NO: 352 - protein, 9 aa, Artificial Sequence
  ligand of HLA-B*08
ILKQKIADL

SEQ ID NO: 353 - protein, 8 aa, Artificial Sequence
  ligand of HLA-B*08
FTKVKPLL

SEQ ID NO: 354 - protein, 9 aa, Artificial Sequence
  ligand of HLA-B*08
YLLEKSRAI

SEQ ID NO: 355 - protein, 8 aa, Artificial Sequence
  ligand of HLA-B*08
EAFVRHIL

SEQ ID NO: 356 - protein, 9 aa, Artificial Sequence
  ligand of HLA-B*08
WVKEKVVAL

SEQ ID NO: 357 - protein, 10 aa, Artificial Sequence
  ligand of HLA-B*08
PASKKTDPQK

SEQ ID NO: 358 - protein, 9 aa, Artificial Sequence
  ligand of HLA-B*08
DIHHKVLSL

SEQ ID NO: 359 - protein, 9 aa, Artificial Sequence
  ligand of HLA-B*08
ELKVKNLEL

SEQ ID NO: 360 - protein, 9 aa, Artificial Sequence
  ligand of HLA-B*08
DAKIRIFDL

SEQ ID NO: 361 - protein, 8 aa, Artificial Sequence
  ligand of HLA-B*08
EPKYKTQL

SEQ ID NO: 362 - protein, 9 aa, Artificial Sequence
  ligand of HLA-B*08
DPMKARVVL

SEQ ID NO: 363 - protein, 8 aa, Artificial Sequence
  ligand of HLA-B*08
DLKQKNEL

SEQ ID NO: 364 - protein, 8 aa, Artificial Sequence
  ligand of HLA-B*08
YGMPRQIL

SEQ ID NO: 365 - protein, 9 aa, Artificial Sequence
  ligand of HLA-B*08
DLERKVESL

SEQ ID NO: 366 - protein, 8 aa, Artificial Sequence
  ligand of HLA-B*08
HPKYKTEL

SEQ ID NO: 367 - protein, 9 aa, Artificial Sequence
  ligand of HLA-B*08
GFKQSSKAL

SEQ ID NO: 368 - protein, 8 aa, Artificial Sequence
  ligand of HLA-B*08
RAKFKQLL

SEQ ID NO: 369 - protein, 9 aa, Artificial Sequence
  ligand of HLA-B*08
FLRGRAYGL

SEQ ID NO: 370 - protein, 9 aa, Artificial Sequence
  ligand of HLA-B*08
QAKWRLQTL

SEQ ID NO: 371 - protein, 9 aa, Artificial Sequence
  ligand of HLA-B*08
AYPLHEQHG

SEQ ID NO: 372 - protein, 9 aa, Artificial Sequence
  ligand of HLA-B*08
YIKSFVSDA

SEQ ID NO: 373 - protein, 9 aa, Artificial Sequence
  ligand of HLA-B*08
HSKKKCDEL

SEQ ID NO: 374 - protein, 8 aa, Artificial Sequence
  ligand of HLA-B*08
WPTVRERM

SEQ ID NO: 375 - protein, 8 aa, Artificial Sequence
  ligand of HLA-B*08
FLKEKGGL

SEQ ID NO: 376 - protein, 8 aa, Artificial Sequence
  ligand of HLA-B*08
WPTVRERM

SEQ ID NO: 377 - protein, 9 aa, Artificial Sequence
  ligand of HLA-B*08
EIKDTKEAL

SEQ ID NO: 378 - protein, 8 aa, Artificial Sequence
  ligand of HLA-B*08
GGKKKYKL

SEQ ID NO: 379 - protein, 9 aa, Artificial Sequence
  ligand of HLA-B*08
ELRSLYNTV

SEQ ID NO: 380 - protein, 9 aa, Artificial Sequence
  ligand of HLA-B*08
EIYKRWIIL

SEQ ID NO: 381 - protein, 9 aa, Artificial Sequence
  ligand of HLA-B*08
GEIYKRWII

SEQ ID NO: 382 - protein, 9 aa, Artificial Sequence
  ligand of HLA-B*08
DCKTILKAL

SEQ ID NO: 383 - protein, 8 aa, Artificial Sequence
  ligand of HLA-B*08
EIYKRWII

SEQ ID NO: 384 - protein, 8 aa, Artificial Sequence
  ligand of HLA-B*08
YLKDQQLL

SEQ ID NO: 385 - protein, 9 aa, Artificial Sequence
  ligand of HLA-B*08
GPKVKQWPL

SEQ ID NO: 386 - protein, 9 aa, Artificial Sequence
  ligand of HLA-B*08
RVKEKYQHL

SEQ ID NO: 387 - protein, 9 aa, Artificial Sequence
  ligand of HLA-B*08
TLHEYMLDL

SEQ ID NO: 388 - protein, 9 aa, Artificial Sequence
  ligand of HLA-B*08
ELRRKMMYM

SEQ ID NO: 389 - protein, 9 aa, Artificial Sequence
  ligand of HLA-B*08
ELRSRYWAI

SEQ ID NO: 390 - protein, 9 aa, Artificial Sequence
  ligand of HLA-B*08
ADRGLLRDI

SEQ ID NO: 391 - protein, 9 aa, Artificial Sequence
  ligand of HLA-B*08
RPIIRPATL

SEQ ID NO: 392 - protein, 9 aa, Artificial Sequence
  ligand of HLA-B*08
LRKPKHKKL

SEQ ID NO: 393 - protein, 9 aa, Artificial Sequence
  ligand of HLA-B*08
ASKNKEKAL

SEQ ID NO: 394 - protein, 8 aa, Artificial Sequence
  ligand of HLA-B*08
LPHNHTDL

SEQ ID NO: 395 - protein, 9 aa, Artificial Sequence
  ligand of HLA-B*35
TSEHSHFSL

SEQ ID NO: 396 - protein, 15 aa, Artificial Sequence
  ligand of HLA-B*35
DDVWTSGSDSDEELV

SEQ ID NO: 397 - protein, 9 aa, Artificial Sequence
  ligand of HLA-B*35
VFPTKDVAL

SEQ ID NO: 398 - protein, 8 aa, Artificial Sequence
  ligand of HLA-B*35
VPLRPMTY

SEQ ID NO: 399 - protein, 9 aa, Artificial Sequence
  ligand of HLA-B*35
TAVPWNASW

SEQ ID NO: 400 - protein, 9 aa, Artificial Sequence
  ligand of HLA-B*35
NSSKVSQNY

SEQ ID NO: 401 - protein, 9 aa, Artificial Sequence
  ligand of HLA-B*35
NPDIVIYQY

SEQ ID NO: 402 - protein, 9 aa, Artificial Sequence
  ligand of HLA-B*35
PPIPVGDIY

SEQ ID NO: 403 - protein, 11 aa, Artificial Sequence
  ligand of HLA-B*35
VPVWKEATTTL

SEQ ID NO: 404 - protein, 10 aa, Artificial Sequence
  ligand of HLA-B*35
VPLDEDFRKY

SEQ ID NO: 405 - protein, 9 aa, Artificial Sequence
  ligand of HLA-B*35
TVLDVGDAY

SEQ ID NO: 406 - protein, 9 aa, Artificial Sequence
  ligand of HLA-B*35
HPDIVIYQY

SEQ ID NO: 407 - protein, 9 aa, Artificial Sequence
  ligand of HLA-B*35
NPVPVGNIY

SEQ ID NO: 408 - protein, 9 aa, Artificial Sequence
  ligand of HLA-B*35
PPSGKGGNY

SEQ ID NO: 409 - protein, 9 aa, Artificial Sequence
  ligand of HLA-B*35
MPNDPNRNV

SEQ ID NO: 410 - protein, 9 aa, Artificial Sequence
  ligand of HLA-B*35
LPIILKALY

SEQ ID NO: 411 - skipped

SEQ ID NO: 412 - protein, 9 aa, Artificial Sequence
  ligand of HLA-B*40
REKIVEVMF

SEQ ID NO: 413 - protein, 9 aa, Artificial Sequence
  ligand of HLA-B*40
LESLDQLEL

SEQ ID NO: 414 - protein, 9 aa, Artificial Sequence
  ligand of HLA-B*40
HEETPPTTS

SEQ ID NO: 415 - protein, 9 aa, Artificial Sequence
  ligand of HLA-B*40
SEDKKNIIL

SEQ ID NO: 416 - protein, 9 aa, Artificial Sequence
  ligand of HLA-B*40
GEVDVEQHT

SEQ ID NO: 417 - protein, 9 aa, Artificial Sequence
  ligand of HLA-B*40
KEPEINTTL

SEQ ID NO: 418 - protein, 9 aa, Artificial Sequence
  ligand of HLA-B*40
HEDKIRVVL

SEQ ID NO: 419 - protein, 12 aa, Artificial Sequence
  ligand of HLA-B*40
GEGDFLAEGGGV

SEQ ID NO: 420 - protein, 9 aa, Artificial Sequence
  ligand of HLA-B*40
GEITGEVRM

SEQ ID NO: 421 - protein, 9 aa, Artificial Sequence
  ligand of HLA-B*40
GEITGEVHM

SEQ ID NO: 422 - protein, 9 aa, Artificial Sequence
  ligand of HLA-B*40
GELTGEVRM

SEQ ID NO: 423 - protein, 8 aa, Artificial Sequence
  ligand of HLA-B*40
TELLIRKL

SEQ ID NO: 424 - protein, 9 aa, Artificial Sequence
  ligand of HLA-B*40
EEFYVDLER

SEQ ID NO: 425 - protein, 9 aa, Artificial Sequence
  ligand of HLA-B*40
GEILDVFDA

SEQ ID NO: 426 - protein, 9 aa, Artificial Sequence
  ligand of HLA-B*40
TEARFGAQL

SEQ ID NO: 427 - protein, 10 aa, Artificial Sequence
  ligand of HLA-B*40
AEIRHVLVTL

SEQ ID NO: 428 - protein, 9 aa, Artificial Sequence
  ligand of HLA-B*40
YEELVRMVL

SEQ ID NO: 429 - protein, 9 aa, Artificial Sequence
  ligand of HLA-B*40
TESGLNVTL

SEQ ID NO: 430 - protein, 10 aa, Artificial Sequence
  ligand of HLA-B*40
IEVDGKQVEL

SEQ ID NO: 431 - protein, 10 aa, Artificial Sequence
  ligand of HLA-B*40
GESDDSILRL

SEQ ID NO: 432 - protein, 9 aa, Artificial Sequence
  ligand of HLA-B*40
SESPIVVVL

SEQ ID NO: 433 - protein, 9 aa, Artificial Sequence
  ligand of HLA-B*40
TEHGVEVVL

SEQ ID NO: 434 - protein, 9 aa, Artificial Sequence
  ligand of HLA-B*40
NEFPEPIKL

SEQ ID NO: 435 - protein, 9 aa, Artificial Sequence
  ligand of HLA-B*40
AEPEEVEVL

SEQ ID NO: 436 - protein, 9 aa, Artificial Sequence
  ligand of HLA-B*40
KESTLHLVL

SEQ ID NO: 437 - protein, 9 aa, Artificial Sequence
  ligand of HLA-B*40
KESTLHLVL

SEQ ID NO: 438 - protein, 9 aa, Artificial Sequence
  ligand of HLA-B*40
REKLQEEML

SEQ ID NO: 439 - protein, 9 aa, Artificial Sequence
  ligand of HLA-B*40
SEVQDRVML

SEQ ID NO: 440 - protein, 9 aa, Artificial Sequence
  ligand of HLA-B*40
KEVNSQLSL

SEQ ID NO: 441 - protein, 10 aa, Artificial Sequence
  ligand of HLA-B*40
RESEEESVSL

SEQ ID NO: 442 - protein, 10 aa, Artificial Sequence
  ligand of HLA-B*44
AEVDKVTGRF

SEQ ID NO: 443 - protein, 9 aa, Artificial Sequence
  ligand of HLA-B*44
TEKVLAAVY

SEQ ID NO: 444 - protein, 9 aa, Artificial Sequence
  ligand of HLA-B*44
TERELKVAY

SEQ ID NO: 445 - protein, 8 aa, Artificial Sequence
  ligand of HLA-B*44
DEVKFLTV

SEQ ID NO: 446 - protein, 8 aa, Artificial Sequence
  ligand of HLA-B*44
DEVRTLTY

SEQ ID NO: 447 - protein, 9 aa, Artificial Sequence
  ligand of HLA-B*44
DEAAFLERL

SEQ ID NO: 448 - protein, 10 aa, Artificial Sequence
  ligand of HLA-B*44
EEIREAFRVF

SEQ ID NO: 449 - protein, 9 aa, Artificial Sequence
  ligand of HLA-B*44
NEQDLGIQY

SEQ ID NO: 450 - protein, 8 aa, Artificial Sequence
  ligand of HLA-B*44
DEVELIHF

SEQ ID NO: 451 - protein, 9 aa, Artificial Sequence
  ligand of HLA-B*44
NENSLFKSL

SEQ ID NO: 452 - protein, 8 aa, Artificial Sequence
  ligand of HLA-B*44
DEFKVVVV

SEQ ID NO: 453 - protein, 8 aa, Artificial Sequence
  ligand of HLA-B*44
LEGLTVVY

SEQ ID NO: 454 - protein, 9 aa, Artificial Sequence
  ligand of HLA-B*44
TENDIRVMF

SEQ ID NO: 455 - protein, 9 aa, Artificial Sequence
  ligand of HLA-B*44
KEHVIQNAF

SEQ ID NO: 456 - protein, 10 aa, Artificial Sequence
  ligand of HLA-B*44
NEFSLKGVDF

SEQ ID NO: 457 - protein, 9 aa, Artificial Sequence
  ligand of HLA-B*44
EEVKLIKKM

SEQ ID NO: 458 - protein, 8 aa, Artificial Sequence
  ligand of HLA-B*44
NELPTVAF

SEQ ID NO: 459 - protein, 8 aa, Artificial Sequence
  ligand of HLA-B*44
VEAIFSKY

SEQ ID NO: 460 - protein, 10 aa, Artificial Sequence
  ligand of HLA-B*44
SEAGSHTLQW

SEQ ID NO: 461 - protein, 9 aa, Artificial Sequence
  ligand of HLA-B*44
EEFGQAFSF

SEQ ID NO: 462 - protein, 9 aa, Artificial Sequence
  ligand of HLA-B*44
VESPLSVSF

SEQ ID NO: 463 - protein, 9 aa, Artificial Sequence
  ligand of HLA-B*44
VEVLLNYAY

SEQ ID NO: 464 - protein, 9 aa, Artificial Sequence
  ligand of HLA-B*44
SEVKFTVTF

SEQ ID NO: 465 - protein, 9 aa, Artificial Sequence
  ligand of HLA-B*44
DERTFHIFY

SEQ ID NO: 466 - protein, 10 aa, Artificial Sequence
  ligand of HLA-B*44
EEVHDLERKY

SEQ ID NO: 467 - protein, 9 aa, Artificial Sequence
  ligand of HLA-B*44
DEVKLPAKL

SEQ ID NO: 468 - protein, 9 aa, Artificial Sequence
  ligand of HLA-B*44
GEAVVNRVF

SEQ ID NO: 469 - protein, 8 aa, Artificial Sequence
  ligand of HLA-B*44
DEGKVIRF

SEQ ID NO: 470 - protein, 9 aa, Artificial Sequence
  ligand of HLA-B*44
KENPLQFKF

SEQ ID NO: 471 - protein, 8 aa, Artificial Sequence
  ligand of HLA-B*44
IETIINTF

SEQ ID NO: 472 - protein, 8 aa, Artificial Sequence
  ligand of HLA-B*44
DEMKVLVL

SEQ ID NO: 473 - protein, 9 aa, Artificial Sequence
  ligand of HLA-B*44
EEASLLHQF

SEQ ID NO: 474 - protein, 9 aa, Artificial Sequence
  ligand of HLA-B*44
DEGRLVLEF

SEQ ID NO: 475 - protein, 9 aa, Artificial Sequence
  ligand of HLA-B*44
KESTLHLVL

SEQ ID NO: 476 - protein, 10 aa, Artificial Sequence
  ligand of HLA-B*44
EEIDLRSVGW

SEQ ID NO: 477 - protein, 10 aa, Artificial Sequence
  ligand of HLA-B*44
NEIEDTFRQF

SEQ ID NO: 478 - protein, 9 aa, Artificial Sequence
  ligand of HLA-B*44
EEIAFLKKL

SEQ ID NO: 479 - protein, 9 aa, Artificial Sequence
  ligand of HLA-B*44
DELELHQRF

SEQ ID NO: 480 - protein, 10 aa, Artificial Sequence
  ligand of HLA-B*44
VEITPYKPTW

SEQ ID NO: 481 - protein, 9 aa, Artificial Sequence
  ligand of HLA-B*44
KEHVIQNAF

SEQ ID NO: 482 - protein, 10 aa, Artificial Sequence
  ligand of HLA-B*44
EENLLDFVRF

SEQ ID NO: 483 - protein, 9 aa, Artificial Sequence
  ligand of HLA-B*44
CEDVPSGKL

SEQ ID NO: 484 - protein, 9 aa, Artificial Sequence
  ligand of HLA-B*44
HERNGFTVL

SEQ ID NO: 485 - protein, 11 aa, Artificial Sequence
  ligand of HLA-B*44
QEFFWDANDIY

SEQ ID NO: 486 - protein, 10 aa, Artificial Sequence
  ligand of HLA-B*44
SEHPTFTSQY

SEQ ID NO: 487 - protein, 9 aa, Artificial Sequence
  ligand of HLA-B*44
NEGLGWAGW

SEQ ID NO: 488 - protein, 9 aa, Artificial Sequence
  ligand of HLA-B*44
AENLWVTVY

SEQ ID NO: 489 - protein, 10 aa, Artificial Sequence
  ligand of HLA-B*44
AENLWVTVYY

SEQ ID NO: 490 - protein, 9 aa, Artificial Sequence
  ligand of HLA-B*44
FEDLRVLSF

SEQ ID NO: 491 - protein, 9 aa, Artificial Sequence
  ligand of HLA-B*44
GEISPLPSL

SEQ ID NO: 492 - protein, 10 aa, Artificial Sequence
  ligand of HLA-B*44
AEMKTDAATL

SEQ ID NO: 493 - protein, 11 aa, Artificial Sequence
  ligand of HLA-B*44
SELFRSGLDSY

SEQ ID NO: 494 - protein, 8 aa, Artificial Sequence
  ligand of HLA-B*51
LPLKMLNI

SEQ ID NO: 495 - protein, 10 aa, Artificial Sequence
  ligand of HLA-B*51
YALPLKMLNI

SEQ ID NO: 496 - protein, 9 aa, Artificial Sequence
  ligand of HLA-B*51
MPFATPMEA

SEQ ID NO: 497 - protein, 9 aa, Artificial Sequence
  ligand of HLA-B*51
VPYGSFKHV

SEQ ID NO: 498 - protein, 10 aa, Artificial Sequence
  ligand of HLA-B*51
DTPLIPLTIF

SEQ ID NO: 499 - protein, 9 aa, Artificial Sequence
  ligand of HLA-B*51
NAHEGQLVI

SEQ ID NO: 500 - protein, 9 aa, Artificial Sequence
  ligand of HLA-B*51
IPLTEEAEL

SEQ ID NO: 501 - protein, 9 aa, Artificial Sequence
  ligand of HLA-B*51
DPNPQEVVL

SEQ ID NO: 502 - protein, 9 aa, Artificial Sequence
  ligand of HLA-B*51
RAIEAQQHL

SEQ ID NO: 503 - protein, 9 aa, Artificial Sequence
  ligand of HLA-B*51
NANPDCKTI

SEQ ID NO: 504 - protein, 9 aa, Artificial Sequence
  ligand of HLA-B*51
TAFTIPSIY

SEQ ID NO: 505 - protein, 12 aa, Artificial Sequence
  ligand of HLA-DPB1*0401
TQHFVQENYLEY

SEQ ID NO: 506 - protein, 13 aa, Artificial Sequence
  ligand of HLA-DQA1*0301/DQB1*0301 (DQ3.1)
LNFDFQAKQLSDP

SEQ ID NO: 507 - protein, 14 aa, Artificial Sequence
  ligand of HLA-DQA1*0301/DQB1*0301 (DQ3.1)
STFDAGAGIALNDH

SEQ ID NO: 508 - protein, 12 aa, Artificial Sequence
  ligand of HLA-DQA1*0301/DQB1*0301 (DQ3.1)
TAADTAAQITQR

SEQ ID NO: 509 - protein, 15 aa, Artificial Sequence
  ligand of HLA-DQA1*0201
FYLLYYTEFTPTEKD

SEQ ID NO: 510 - protein, 13 aa, Artificial Sequence
  ligand of HLA-DQA1*0201
LPSTEDVYDCRVE

SEQ ID NO: 511 - protein, 15 aa, Artificial Sequence
  ligand of HLA-DQA1*0201
TEDVYDCRVEHWGLD

SEQ ID NO: 512 - protein, 16 aa, Artificial Sequence
  ligand of HLA-DQA1*0201
EPRAPWIEQEGPEYWD

SEQ ID NO: 513 - protein, 20 aa, Artificial Sequence
  ligand of HLA-DQA1*0201
LPKPPKPVSKMRMATPLLMQ

SEQ ID NO: 514 - protein, 21 aa, Artificial Sequence
  ligand of HLA-DQA1*0201
LRSLDRNLPSDSQDLGQHGLE

SEQ ID NO: 515 - protein, 16 aa, Artificial Sequence
  ligand of HLA-DRB1*03
IPENLFLKSDGRVKYT

SEQ ID NO: 516 - protein, 14 aa, Artificial Sequence
  ligand of HLA-DRB1*03
EPLPLKVNEQRKAV

SEQ ID NO: 517 - protein, 14 aa, Artificial Sequence
  ligand of HLA-DRB1*03
QPPKLLIYWASTRE

SEQ ID NO: 518 - protein, 12 aa, Artificial Sequence
  ligand of HLA-DRB1*03
LKIDLANRETSI

SEQ ID NO: 519 - protein, 12 aa, Artificial Sequence
  ligand of HLA-DRB1*03
PTLVPIEYKNMI

SEQ ID NO: 520 - protein, 20 aa, Artificial Sequence
  ligand of HLA-DRB1*03
RHMGSDWSIEIDGKKYTAQE

SEQ ID NO: 521 - protein, 20 aa, Artificial Sequence
  ligand of HLA-DRB1*03
DSDKNPLFLDEQLIRAEFQR

SEQ ID NO: 522 - protein, 20 aa, Artificial Sequence
  ligand of HLA-DRB1*03
QPSVQIQVYQGEREIASHNK

SEQ ID NO: 523 - protein, 15 aa, Artificial Sequence
  ligand of HLA-DRB1*03
IQMISEAARFQYIEG

SEQ ID NO: 524 - protein, 15 aa, Artificial Sequence
  ligand of HLA-DRB1*03
WEKMKASEKIFYVYM

SEQ ID NO: 525 - protein, 14 aa, Artificial Sequence
  ligand of HLA-DRB1*11
PTFTSQYRIQGKLE

SEQ ID NO: 526 - protein, 15 aa, Artificial Sequence
  ligand of HLA-DRB1*11
IQMISEAARFQYIEG

SEQ ID NO: 527 - protein, 15 aa, Artificial Sequence
  ligand of HLA-DRB1*11
KIFYVYMKRKYEAMT

SEQ ID NO: 528 - protein, 25 aa, Artificial Sequence
  ligand of HLA-DRB1*11
IVHSATGFKQSSKALQRPVASDFEP

SEQ ID NO: 529 - protein, 25 aa, Artificial Sequence
  ligand of HLA-DRB1*11
NSNHVASGAGEAAIETQSSSSEEIV

SEQ ID NO: 530 - protein, 16 aa, Artificial Sequence
  ligand of HLA-DRB1*0301
VFLLLLADKVPETSLS

SEQ ID NO: 531 - protein, 16 aa, Artificial Sequence
  ligand of HLA-DRB1*0301
VDTFLEDVKNLYHSEA

SEQ ID NO: 532 - protein, 17 aa, Artificial Sequence
  ligand of HLA-DRB1*0301
VTTLNSDLKYNALDLTN

SEQ ID NO: 533 - protein, 17 aa, Artificial Sequence
  ligand of HLA-DRB1*0301
ISNQLTLDSNTKYFHKL

SEQ ID NO: 534 - protein, 16 aa, Artificial Sequence
  ligand of HLA-DRB1*0301
ISNQLTLDSNTKYFHK

SEQ ID NO: 535 - protein, 17 aa, Artificial Sequence
  ligand of HLA-DRB1*0301
YANILLDRRVPQTDMTF

SEQ ID NO: 536 - protein, 20 aa, Artificial Sequence [sequence omitted]

SEQ ID NO: 537 - protein, 20 aa, Artificial Sequence
  ligand of HLA-DRB1*0301
IPDNLFLKSDGRIKYTLNKN

SEQ ID NO: 538 - protein, 19 aa, Artificial Sequence
  ligand of HLA-DRB1*0301
IPDNLFLKSDGRIKYTLNK

SEQ ID NO: 539 - protein, 18 aa, Artificial Sequence
  ligand of HLA-DRB1*0301
IPDNLFLKSDGRIKYTLN

SEQ ID NO: 540 - protein, 17 aa, Artificial Sequence
  ligand of HLA-DRB1*0301
IPDNLFLKSDGRIKYTL

SEQ ID NO: 541 - protein, 16 aa, Artificial Sequence
  ligand of HLA-DRB1*0301
NLFLKSDGRIKYTLNK

SEQ ID NO: 542 - protein, 15 aa, Artificial Sequence
  ligand of HLA-DRB1*0301
NLFLKSDGRIKYTLN

SEQ ID NO: 543 - protein, 18 aa, Artificial Sequence
  ligand of HLA-DRB1*0301
GKFAIRPDKKSNPIIRTV

SEQ ID NO: 544 - protein, 15 aa, Artificial Sequence
  ligand of HLA-DRB1*0301
VGSDWRFLRGYHQYA

SEQ ID NO: 545 - protein, 16 aa, Artificial Sequence
  ligand of HLA-DRB1*0301
VDDTQFVRFDSDAASQ

SEQ ID NO: 546 - protein, 13 aa, Artificial Sequence
  ligand of HLA-DRB1*0301
LNKILLDEQAQWK

SEQ ID NO: 547 - protein, 20 aa, Artificial Sequence
  ligand of HLA-DRB1*0301
GPPKLDIRKEEKQIMIDIFH

SEQ ID NO: 548 - protein, 21 aa, Artificial Sequence
  ligand of HLA-DRB1*0301
GPPKLDIRKEEKQIMIDIFHP

SEQ ID NO: 549 - protein, 12 aa, Artificial Sequence
  ligand of HLA-DRB1*0301
KQTISPDYRNMI

SEQ ID NO: 550 - protein, 17 aa, Artificial Sequence
  ligand of HLA-DRB1*0301
LPKPPKPVSKMRMATPL

SEQ ID NO: 551 - protein, 23 aa, Artificial Sequence
  ligand of HLA-DRB1*0301
LPKPPKPVSKMRMATPLLMQALP

SEQ ID NO: 552 - protein, 23 aa, Artificial Sequence
  ligand of HLA-DRB1*0301
LPKPPKPVSKMRMATPLLMQALP

SEQ ID NO: 553 - protein, 16 aa, Artificial Sequence
  ligand of HLA-DRB1*0301
PKPPKPVSKMRMATPL

SEQ ID NO: 554 - protein, 20 aa, Artificial Sequence
  ligand of HLA-DRB1*0301
PKPPKPVSKMRMATPLLMQA

SEQ ID NO: 555 - protein, 18 aa, Artificial Sequence
  ligand of HLA-DRB1*0301
KPPKPVSKMRMATPLLMQ

SEQ ID NO: 556 - protein, 22 aa, Artificial Sequence
  ligand of HLA-DRB1*0301
KPPKPVSKMRMATPLLMQALPM

SEQ ID NO: 557 - protein, 19 aa, Artificial Sequence
  ligand of HLA-DRB1*0301
ATKYGNMTEDHVMHLLQNA

SEQ ID NO: 558 - protein, 15 aa, Artificial Sequence
  ligand of HLA-DRB1*0301
KPRAIVVDPVHGFMY

SEQ ID NO: 559 - protein, 15 aa, Artificial Sequence
  ligand of HLA-DRB1*0301
LLSFVRDLNQYRADI

SEQ ID NO: 560 - protein, 20 aa, Artificial Sequence
  ligand of HLA-DRB1*0301
GDVVAVDIKEKGKDKWIELK

SEQ ID NO: 561 - protein, 10 aa, Artificial Sequence
  ligand of HLA-DRB1*0301
MGRSIKVQLQ

SEQ ID NO: 562 - protein, 13 aa, Artificial Sequence
  ligand of HLA-DRB1*0301
SDKNPLFLDEQLI

SEQ ID NO: 563 - protein, 11 aa, Artificial Sequence
  ligand of HLA-DRB1*0301
KTIAYDEEARR

SEQ ID NO: 564 - protein, 18 aa, Artificial Sequence
  ligand of HLA-DRB1*0301
YLQVPSPSMGRDIKVQFQ

SEQ ID NO: 565 - protein, 15 aa, Artificial Sequence
  ligand of HLA-DRB1*0301
TIRLTAADHRQLQLS

SEQ ID NO: 566 - protein, 15 aa, Artificial Sequence
  ligand of HLA-DRB1*0301
WEKMKASEKIFYVYM

SEQ ID NO: 567 - protein, 21 aa, Artificial Sequence
  ligand of HLA-DRB1*0701
VTQYLNATGNRWCSWSLSQAR

SEQ ID NO: 568 - protein, 17 aa, Artificial Sequence
  ligand of HLA-DRB1*0701
VTQYLNATGNRWCSWSL

SEQ ID NO: 569 - protein, 15 aa, Artificial Sequence
  ligand of HLA-DRB1*0701
KVDLTFSKQHALLCS

SEQ ID NO: 570 - protein, 13 aa, Artificial Sequence
  ligand of HLA-DRB1*0701
FSHDYRGSTSHRL

SEQ ID NO: 571 - protein, 13 aa, Artificial Sequence
  ligand of HLA-DRB1*0701
LPKYFEKKRNTII

SEQ ID NO: 572 - protein, 19 aa, Artificial Sequence
  ligand of HLA-DRB1*0701
APVLISQKLSPIYNLVPVK

SEQ ID NO: 573 - protein, 18 aa, Artificial Sequence
  ligand of HLA-DRB1*0701
VPGLYSPCRAFFNKEELL

SEQ ID NO: 574 - protein, 14 aa, Artificial Sequence
  ligand of HLA-DRB1*0701
VPGLYSPCRAFFNK

SEQ ID NO: 575 - protein, 18 aa, Artificial Sequence
  ligand of HLA-DRB1*0701
VGSDWRFLRGYHQYAYDG

SEQ ID NO: 576 - protein, 20 aa, Artificial Sequence
  ligand of HLA-DRB1*0701
RPAGDGTFQKWASVVVPSGQ

SEQ ID NO: 577 - protein, 16 aa, Artificial Sequence
  ligand of HLA-DRB1*0701
RPAGDGTFQKWASVVV

SEQ ID NO: 578 - protein, 15 aa, Artificial Sequence
  ligand of HLA-DRB1*0701
GDGTFQKWASVVVPS

SEQ ID NO: 579 - protein, 14 aa, Artificial Sequence
  ligand of HLA-DRB1*0701
GTFQKWASVVVPSG

SEQ ID NO: 580 - protein, 15 aa, Artificial Sequence
  ligand of HLA-DRB1*0701
GTFQKWASVVVPSGQ

SEQ ID NO: 581 - protein, 23 aa, Artificial Sequence
  ligand of HLA-DRB1*0701
GTFQKWASVVVPSGQEQRYTCHV

SEQ ID NO: 582 - protein, 15 aa, Artificial Sequence
  ligand of HLA-DRB1*0701
RETQISKTNTQTYRE

SEQ ID NO: 583 - protein, 15 aa, Artificial Sequence
  ligand of HLA-DRB1*0701
RETQISKTNTQTYRE

SEQ ID NO: 584 - protein, 15 aa, Artificial Sequence
  ligand of HLA-DRB1*0701
RETQISKTNTQTYRE

SEQ ID NO: 585 - protein, 30 aa, Artificial Sequence
  ligand of HLA-DRB1*0701
SLQSPITVEWRAQSESAQSKMLSGIGGFVL

SEQ ID NO: 586 - protein, 26 aa, Artificial Sequence
  ligand of HLA-DRB1*0701
RSNYTPITNPPEVTVLTNSPVELREP

SEQ ID NO: 587 - protein, 21 aa, Artificial Sequence
  ligand of HLA-DRB1*0701
GALANIAVDKANLEIMTKRSN

SEQ ID NO: 588 - protein, 20 aa, Artificial Sequence
  ligand of HLA-DRB1*0701
APSPLPETTENVVCALGLTV

SEQ ID NO: 589 - protein, 17 aa, Artificial Sequence
  ligand of HLA-DRB1*0701
TPSYVAFTDTERLIGDA

SEQ ID NO: 590 - protein, 15 aa, Artificial Sequence
  ligand of HLA-DRB1*0701
TPSYVAFTDTERLIG

SEQ ID NO: 591 - protein, 14 aa, Artificial Sequence
  ligand of HLA-DRB1*0701
KHKVYACEVTHQGL

SEQ ID NO: 592 - protein, 22 aa, Artificial Sequence
  ligand of HLA-DRB1*0701
PKPPKPVSKMRMATPLLMQALP

SEQ ID NO: 593 - protein, 25 aa, Artificial Sequence
  ligand of HLA-DRB1*0701
GDMYPKTWSGMLVGALCALAGVLTI

SEQ ID NO: 594 - protein, 13 aa, Artificial Sequence
  ligand of HLA-DRB1*0701
TSILCYRKREWIK

SEQ ID NO: 595 - protein, 15 aa, Artificial Sequence
  ligand of HLA-DRB1*0701
PAFRFTREAAQDCEV

SEQ ID NO: 596 - protein, 23 aa, Artificial Sequence
  ligand of HLA-DRB1*0701
SAYLAHRNQSLDLAEQELVDCAS

SEQ ID NO: 597 - protein, 25 aa, Artificial Sequence
  ligand of HLA-DRB1*0701
QIYPPNANKIREALAQTHSAIAHWT

SEQ ID NO: 598 - protein, 20 aa, Artificial Sequence
  ligand of HLA-DRB1*0701
PGPLRESIVCYFMVFLQTHI

SEQ ID NO: 599 - protein, 15 aa, Artificial Sequence
  ligand of HLA-DRB1*0701
KALENKKKQLGAGGK

SEQ ID NO: 600 - protein, 19 aa, Artificial Sequence
  ligand of HLA-DRB1*0701
FRKQNPDIVIQYMDDLYVG

SEQ ID NO: 601 - protein, 20 aa, Artificial Sequence
  ligand of HLA-DRB1*0701
AKIELSSSQSTSVNLPYITV

SEQ ID NO: 602 - protein, 16 aa, Artificial Sequence
  ligand of HLA-DRB1*0701
RPGLLGASVLGDDIHT

SEQ ID NO: 603 - protein, 19 aa, Artificial Sequence
  ligand of HLA-DRB1*0701
PKYVKQNTLKLATGMRNVP

SEQ ID NO: 604 - protein, 15 aa, Artificial Sequence
  ligand of HLA-DRB1*0701
RMPTVLQCVNVSVVS

SEQ ID NO: 605 - protein, 15 aa, Artificial Sequence
  ligand of HLA-DRB1*0701
AEGLRALLARSHVER

SEQ ID NO: 606 - protein, 15 aa, Artificial Sequence
  ligand of HLA-DRB1*0701
TIRLTAADHRQLQLS

SEQ ID NO: 607 - protein, 20 aa, Artificial Sequence
  ligand of HLA-DRB1*0701
EYLNKIQNSLSTEWSPCSVT

SEQ ID NO: 608 - protein, 15 aa, Artificial Sequence
  ligand of HLA-DRB1*0701
DALESIMTTKSVSFR

SEQ ID NO: 609 - protein, 15 aa, Artificial Sequence
  ligand of HLA-DRB1*0701
ALTGGMELTRDPTVP

SEQ ID NO: 610 - protein, 15 aa, Artificial Sequence
  ligand of HLA-DRB1*0701
KIFYVYMKRKYEAMT

SEQ ID NO: 611 - protein, 15 aa, Artificial Sequence
  ligand of HLA-DRB1*1501
EAEQLRAYLDGTGVE

SEQ ID NO: 612 - protein, 14 aa, Artificial Sequence
  ligand of HLA-DRB1*1501
LEEFGRFASFEAQG

SEQ ID NO: 613 - protein, 11 aa, Artificial Sequence
  ligand of HLA-DRB1*1501
YAYVAREQSCR

SEQ ID NO: 614 - protein, 19 aa, Artificial Sequence
  ligand of HLA-DRB1*1501
AGLTLSLLVICSYLFISRG

SEQ ID NO: 615 - protein, 9 aa, Artificial Sequence
  ligand of HLA-DRB1*1501
VIGLYGNGV

SEQ ID NO: 616 - protein, 15 aa, Artificial Sequence
  ligand of HLA-DRB1*1501
MSIYVYALPLKMLNI

SEQ ID NO: 617 - protein, 20 aa, Artificial Sequence
  ligand of HLA-DRB1*1501
GRHLIFCHSKRKCDELATKL

SEQ ID NO: 618 - protein, 13 aa, Artificial Sequence
  ligand of HLA-DRB1*1501
AHYNIVTFCCKCD

SEQ ID NO: 619 - protein, 12 aa, Artificial Sequence
  ligand of HLA-DRB1*1501
RVVINKDTTIII

SEQ ID NO: 620 - protein, 11 aa, Artificial Sequence
  ligand of HLA-DRB1*1501
PVVHFFKNIVT

SEQ ID NO: 621 - protein, 20 aa, Artificial Sequence
  ligand of HLA-DRB1*1501
SKYLATASTMDHARHGTLPR

SEQ ID NO: 622 - protein, 15 aa, Artificial Sequence
  ligand of HLA-DRB1*1501
AEGLRALLARSHVER

SEQ ID NO: 623 - protein, 20 aa, Artificial Sequence
  ligand of HLA-DRB1*1501
WTTCQSIAFPSKTSASIGSL

SEQ ID NO: 624 - protein, 20 aa, Artificial Sequence
  ligand of HLA-DRB1*1501
LFCGCGHEALTGTEKLIETY

SEQ ID NO: 625 - protein, 21 aa, Artificial Sequence
  ligand of HLA-DRB1*1501
RHRPLQEVYPEANAPIGHNRE

SEQ ID NO: 626 - protein, 9 aa, Artificial sequence
  peptide
DEYIYRHFE

(19) United States
(12) Patent Application Publication
(10) Pub. No.: US 2011/0039756 A1
(43) Pub. Date: Feb. 17, 2011

(54) MHC RAPID ASSAY USED FOR THE CUSTOMIZATION OF ODOURS

(75) Inventors: August Hämmerli, Bern (CH); Manuel Kaegi, Bern (CH); Dominic Senn, Bern (CH)

Correspondence Address: SMITH PATENT CONSULTING, LLC, 515 East Braddock Road, Suite B, ALEXANDRIA, VA, 22314, US

(73) Assignee: basisnote AG, Bern (CH)

(21) Appl. No.: 12/809,686

(22) PCT Filed: Dec. 19, 2008
(86) PCT No.: PCT/EP2008/010898

§ 371 Date: Jun. 21, 2010

(30) Foreign Application Priority Data
Dec. 21, 2007 (EP) 07025035.2

(51) Int. Cl. A61K 8/64; A61Q 13/00

(52) U.S. Cl. 512/26

(57) ABSTRACT

The present invention provides a user friendly test kit for determining an individual's Major Histocompatibilty profile and using that profile for the customization of odours.

### FIELD OF THE INVENTION

The present invention relates to the rapid, non-diagnostic, non-therapeutic assay for MHC or MHC-correlated profiles and to the matching of such profiles as a means of determining optimal mating compatibility between individuals. Particularly, the invention is applicable for the encrypted and anonymous matching of optimal mating partners in a digital format as may be applicable in such fields as internet-based dating services. The invention includes the customization of odours (fragrances, cosmetic products) for animals, humans, objects, interior and exterior spaces based on the results of the MHC rapid assay.

### BACKGROUND OF THE INVENTION

The major histocompatibility (MHC) gene complex encodes cell-surface glycoproteins (class I and II molecules) that bind peptides and present them to T lymphocytes [1]. While the MHC complex has been detected in the field of medicine, specifically in transplantation medicine, the high degree of heterozygosity in this gene complex found in natural populations of most species is most likely promoted by non-disease-based selection such as mating preferences [2]. Evolutionary biologists have been investigating MHC-dependent mating preferences in vertebrates [3] and particularly in humans [4]. Evidence is accumulating that in many species, MHC genes influence reproductive behaviour and body odour, generating MHC-correlated olfactory cues for potential mating partners [3, 5]. The high diversity of MHC genes may be responsible for the distinctive scent of individuals, which is analogous to a signature or fingerprint [6]. Such MHC-correlated odour fingerprints consist of various volatile odour compounds and MHC peptide ligands [7].

Empirically, in the three spined stickleback fish, evolutionarily conserved structural features of MHC peptide ligands were used to evaluate MHC diversity of prospective mating partners through MHC-correlated olfactory cues [8]. In human behaviour, odour cues provide information regarding the degree of relatedness and may thus affect kin preferences and mate choice [9]. For example, it has been shown that humans can influence the hormonal balance of conspecifics through chemosignals from sweat [10]. Evidence is accumulating that olfactory cues on MHC status of individuals are also possibly enhanced through the use of perfumes. Individual preferences for fragrances seem to be determined in some way by amplifying one's own body odour that reveals a person's immunogenetics [11]. Odour cues that reveal the immunogenetic status of individuals are highly resolved. Women for example have been shown to be able to differentiate a single allele difference among male odour donors with different MHC genotypes [12]. MHC-dependent mate preferences through odour cues may even influence the psychology of sexual attraction and the number of extra-pair sexual partners in relationships [13]. Evolutionary biologist assume that the optimal MHC complement for a partner should include those alleles that provide resistance against the parasites in the current environment [14]. Theoreticians have predicted two opposing forces, namely parasite resistance and inbreeding avoidance, to result in an optimal number of genes at intermediate individual MHC diversity [15, 16]. This prediction is now supported both by experimental data [17] and a population genetic survey [18] with the three spined stickleback.

MHC linked odour cues appear at the periphery via different pathways. Soluble MHC molecules for example are known that carry allele-specific odoriferous molecules from the blood via the kidneys into the urine [5] and such molecules have been identified in the urine proteome [19], where carboxylic acids are most likely to be the odour-components linked to MHC [20]. More and more data is becoming available on odour components not only in urine but also sweat and saliva and many of these components seem to be MHC determined [6]. Volatile odorant components have been experimentally shown to activate neurons in the vomeronasal organ [21]. And among those, small peptides that serve as ligands for MHC molecules function as sensory stimuli [22]. Fish, rodents and humans have been shown to be able to differentiate particular MHC profiles by smell [20] and such differences could in principle even be detected with artificial screening technology (electronic nose) [23] [24]. Data on these ligands is rapidly accumulating [25] together with their specific binding properties to MHC molecules [26].

US patent application 2005/0112684 A1 proposes a method of matching human beings, which comprises collecting genetic sample material from individuals and subsequently determining the individuals' MHC profiles in a genetic laboratory. Similarly, US 2007/069889 A1 and US 2007/069901 disclose matching services which may include the determination of MHC profiles. These methods, however, require that the individuals give their genetic material to an institution where it is analyzed. It is likely that the users or participants will be reluctant to give their genetic material and have it analyzed at a location where it is beyond their control.

US patent application 2007/0243537 A1 discloses a human sample matching system. This reference describes a method comprising the steps of collecting a DNA sample from each of a plurality of human individuals, determining a set of genetic attributes for each of said human individuals; and producing a perfume for one of said human individuals based on one of said sets of genetic attributes. US 2007/0243537 A1, however, always requires determination of the genetic attributes on the nucleic acid level, not on the protein level.

EP 1358869 A1 generally discloses the use of MHC ligands for the manufacture of an agent for modifying bodily odours. It does not disclose the selection of the MHC ligands on the basis of an individual's MHC profile.

### SUMMARY OF THE INVENTION

Accordingly, it is one of the objects of the present invention to provide a user-friendly test kit for the rapid determination of an individual's Major Histocompatibility Complex (MHC) profile and for the use of such profiles for customizing odours, comprising:
at least one solid substrate in contact with a proximal sample application zone, said substrate having N conjugation zones and N distal detection zones, said conjugation zones respectively containing N labelled primary binding reagents capable of binding with an analyte to form an analyte-primary binding reagent conjugate; said distal detection zone respectively having immobilized thereunto, N unlabeled secondary binding reagents capable of binding to a migrating analyte-labeled primary binding reagent conjugate to determine the presence or absence of N analytes which are indicative of defined N-MHC antigens, wherein N is an integer greater than 3, preferably from 6 to 30.

Another object of the invention relates to a method for the preparation of one or more customized odour compositions, comprising the following steps:
(a) providing or determining an MHC profile of one or more individuals, wherein said MHC profile contains information on the presence or absence of N different MHC antigens in each individual, wherein N is an integer greater than 3;
(b) for each individual, selecting at least one peptide ligand of an MHC antigen found to be expressed by said individual and/or selecting at least one peptide ligand of an MHC antigen found not to be expressed by said individual;
(c) for each individual, providing at least one compound comprising said at least one peptide ligand selected in step (b), or a fragment or derivative thereof;
(d) for each individual, mixing said at least one compound with a cosmetically acceptable excipient, diluent and/or vehicle to obtain said customized odour composition.

Yet another object of this invention is a method of matching individuals, comprising the following steps:
(a) obtaining a plurality of personal profiles from a plurality of individuals and storing said personal profiles in a database on a computer.
(b) providing a plurality of test kits to said plurality of individuals, wherein each test kit comprises a unique identifier;
(c) obtaining a plurality of encrypted MHC profiles and the respective unique identifiers from said plurality of individuals;
(d) storing said encrypted MHC profiles and the respective unique identifier in a database on a computer, wherein each encrypted MHC profile and its unique identifier is allocated to one of said personal profiles;
(e) decrypting said encrypted MHC profiles using a key related to said unique identifier, and a computer, and storing the decrypted MHC profiles in a database on a computer;
(f) calculating the pair wise distances in decrypted MHC profiles between all individuals for which decrypted MHC profiles have been stored in the database;
(g) determining the quality of match between said individuals from the pair wise distances calculated in step (f).

Another object of the invention relates to a method for the rapid determination of an encrypted MHC profile of an individual in which a test kit according to the invention is contacted with a body liquid sample containing the analyte, such that the sample permeates by capillary action from a sample application zone through the solid substrate via N conjugation zones into N detection zones, and N labelled primary binding reagents migrate with the analyte from the respective conjugation zones to the respective detection zones containing immobilized N unlabelled secondary binding reagents, the presence of analytes in the sample being determined by observing the extent to which the labelled reagents become bound in the detection zones.

Another object of the invention is the customization of odours based on MHC profiles comprising the following steps:
(a) obtaining a plurality of MHC profiles from a plurality of individuals.
(b) using said plurality of MHC profiles for the customization of odours for “enhance” and “complement” as defined in the detailed description below, based on detected analytes.
(c) Providing a plurality of said customized odours to said plurality of individuals for their use to upgrade fragrances, cosmetic products, interior and exterior spaces, surfaces such as textiles or the direct application to enhance body odour, or the application in liquid, solid or gaseous material.

It is to be understood that both the foregoing general description and the following detailed description are exemplary and explanatory and are intended to provide further explanation of the invention as claimed. Other objects and features of the invention will become apparent from the following detailed description considered in conjunction with the accompanying drawings and tables. The accompanying drawings are included solely for purposes of illustration and not as a definition of the limits of the invention. Also, the drawings are not drawn to scale, and are merely conceptual in disclosing the preferred embodiments of the invention.

### BRIEF DESCRIPTION OF THE DRAWINGS

FIG. 1 illustrates a process flow-chart of the processes according to one embodiment of the present invention.

FIG. 2a illustrates one embodiment of a lateral flow-through assay device for rapid assay to detect the encrypted MHC profile of a user.

FIG. 2b illustrates how the result from such a flow-through assay device can be added to a personal profile in an online electronic form together with a unique identifier.

FIG. 3 is a process chart of matching and dating services according to one embodiment of the present invention showing in chronological order, the preferred steps usable to provide a user with a list of potential dating partners that match said user's MHC profile.

FIG. 4 is an object-oriented view of matching and dating services according to one embodiment of the present invention showing the objects, methods and database tables for providing a user with a list of potential dating partners that match said user's MHC profile.

FIG. 5 is a chart illustrating the probability pᵢ with which a test field shows a positive result and the probability p, that two users have the same MHC profile for different number of test fields on the lateral flow assay N={1 . . . 10} in accordance with one embodiment of the present invention.

FIG. 6 depicts in a general manner three different embodiments of the test kit of the invention. The first embodiment is depicted at the top of FIG. 6 and corresponds to a test kit in the “sandwich format”. Two different embodiments of the “competitive format” are depicted in the second and third row of FIG. 6, respectively. The sample (e.g. saliva or urine) containing an analyte is applied to the proximal sample application zone of a test strip. The sample containing the analyte then migrates into the distal direction through the conjugation zone or release zone, respectively, where either anti-analyte antibody or labelled analyte may be deposited. At the distal detection zone capture antibodies or analyte may be immobilized. In the further distal control zone control agents are immobilized capable of binding to the labelled antibody or labelled analyte, respectively. Excess liquid may be absorbed by a most distal absorbent pad. The embodiments depicted in FIG. 6 are in no way limiting but only illustrate certain forms of the subject matter claimed. The various zones and agents shown are not drawn to scale.

### DETAILED DESCRIPTION OF THE INVENTION

### Test Kit

The test kit according to the present invention allows for a user-friendly rapid determination of an individual's MHC profile. The term “rapid” as used herein refers to the time span between the application of a test sample and the availability of the assay result, which may range from about 1 second to about 30 minutes, preferably from about 1 minute to about 20 minutes, more preferably from about 2 minutes to about 15 minutes, most preferably from about 3 to about 8 minutes. The rapid assay can in principle be any device, kit or test format that allows the detection of MHC components within the above mentioned time span. For instance, the rapid assay may be a device based on lateral flow immunochemical technique, semiconductor technique, or any other technical device able to rapidly detect MHC compounds or a correlate thereof.

The term “user-friendly” as used herein refers to the non-requirement of any specialized skills or technical training in order to use the test kit of the invention. The test kit of the invention is suitable for use in home and is intended to give a result which is rapid and which requires the minimum degree of skill and involvement from the user. The test kit preferably requires that some portion of the kit is contacted with the sample (e.g. a urine stream or saliva) and thereafter, no further actions are required by the user before a result can be observed. Ideally, the analytical result is observable within a matter of minutes following sample application, e.g. 30 minutes or less, preferably 10 minutes or less.

The test kit of the invention is suitable for rapidly determining an individual's MHC profile. The term “MHC profile” as used herein refers to a pattern consisting of the presence or absence of a plurality of different MHC antigens expressed by an individual.

The terms “MHC antigen” (Major Histocompatibility Antigen) and “HLA antigen” (human leukocyte-associated antigen) are used interchangeably herein. Unless specified otherwise herein, the term “MHC antigen” refers to serologically defined HLA antigens. MHC antigens in the sense of this invention include, but are not limited to, HLA-A, -B, -C, -DR and -DQ antigens as defined in [40] in Tables 1 to 6 as “WHO assigned type”. The term “MHC antigens” further includes HLA antigens corresponding to the alleles listed in the Scientific Database at http://www.anthonvnolan.org.uk/HIG/lists/class1list.html or http://www.anthonynolan.org.uk/HIG/lists/class2list.html. Suitable MHC antigens may also be found in [45] or at the URL http://www.allelefrequencies.net. The designations of MHC antigens with and without the prefix “HLA-” are used interchangeably herein. For example, the term “DRB1*0403” is to be understood as meaning “HLA- DRB1*0403” and the like.

The MHC profile determined in accordance with this invention may contain information on N different MHC antigens, wherein N is an integer greater than 3. The inventors have found that it is possible to obtain sufficient information on an individual's MHC profile by analyzing a limited number of MHC antigens. Accordingly, N is preferably an integer from 4 to 40, more preferably from 5 to 33, more preferably from 6 to 30, more preferably from 6 to 25, even more preferably from 7 to 20, most preferably from 8 to 15, or from 8 to 12 (e.g. 8, 9, 10, 11 or 12).

In particular, the inventors applied a unique frequency-based selection technique for selecting suitable MHC antigens to be included in the MHC profile. In one embodiment, the set of MHC antigens to be determined includes N different MHC antigens the average phenotype frequency of which is from about 5% to about 95%, preferably from about 10% to about 90%. More preferably, the average frequency of the N different MHC antigens is from about 15% to about 85%, more preferably from about 20% to about 80%, more preferably from about 25% to about 75%, most preferably from about 30% to about 70%. These percentages refer to the average phenotypic frequencies of the respective MHC antigens in all investigated populations, as of the priority date of this application. These groups of MHC antigens are listed in the following.

Based on data from [45], the following MHC antigens were found to be present at an average frequency of from 5% to 95%, as of the priority date of this application:

A*01; A*02; A*0201; A*03; A*11; A*1101; A*24; A*2402; B*07; B*0702; B*08; B*0801; B*13; B*1301; B*14; B*1402; B*15; B*1501; B*1525; B*18; B*1801; B*27; B*35; B*3501; B*38; B*39; B*3901; B*40; B*4001; B*4002; B*44; B*4402; B*4403; B*48; B*4801; B*49; B*51; B*5101; B*52; B*55; B*5502; B*57; B*58; B*5801; Cw*01; Cw*0102; Cw*02; Cw*03; Cw*0303; Cw*0304; Cw*030401; Cw*04; Cw*0401; Cw*0403; Cw*05; Cw*0501; Cw*06; Cw*0602; Cw*07; Cw*0701; Cw*0702; Cw*08; Cw*0801; Cw*12; Cw*1202; Cw*1203; Cw*15; Cw*16; Cw*1601; DPB1*0101; DPB1*0201; DPB1*0301; DPB1*0401; DPB1*0402; DPB1*0501; DPB1*1301; DPB1*1401; DQA1*0101; DQA1*0102; DQA1*0103; DQA1*0104; DQA1*0201; DQA1*03; DQA1*0301; DQA1*0401; DQA1*05; DQA1*0501; DQB1*02; DQB1*0201; DQB1*0202; DQB1*0301; DQB1*0302; DQB1*0303; DQB1*0402; DQB1*0501; DQB1*0502; DQB1*0503; DQB1*06; DQB1*0601; DQB1*0602; DQB1*0603; DQB1*0604; DRB1*01; DRB1*0101; DRB1*0102; DRB1*03; DRB1*0301; DRB1*04; DRB1*0401; DRB1*0403; DRB1*0404; DRB1*0405; DRB1*0406; DRB1*07; DRB1*0701; DRB1*08; DRB1*0802; DRB1*080302; DRB1*09; DRB1*090102; DRB1*11; DRB1*1101; DRB1*110101; DRB1*1104; DRB1*12; DRB1*1201; DRB1*120201; DRB1*13; DRB1*1301; DRB1*1302; DRB1*14; DRB1*1401; DRB1*1405; DRB1*15; DRB1*1501; DRB1*150101; DRB1*1502; DRB1*16; DRB1*1601; and DRB1*160201. This group of MHC antigens is referred to hereinafter as “5/95 group of MHC antigens”.

Based on data from [45], the following MHC antigens were found to be present at an average frequency of from 10% to 90%, as of the priority date of this application:

A*01; A*02; A*0201; A*03; A*11; A*1101; A*24; A*2402; B*07; B*0702; B*08; B*0801; B*13; B*1301; B*14; B*15; B*1525; B*18; B*1801; B*35; B*3501; B*39; B*3901; B*40; B*4001; B*4002; B*44; B*4402; B*4403; B*48; B*4801; B*51; B*5101; B*55; B*5502; B*58; B*5801; Cw*01; Cw*0102; Cw*03; Cw*0303; Cw*0304; Cw*030401; Cw*04; Cw*0401; Cw*0403; Cw*05; Cw*0501; Cw*06; Cw*0602; Cw*07; Cw*0701; Cw*0702; Cw*08; Cw*0801; Cw*12; Cw*1202; Cw*1203; Cw*16; DPB1*0101; DPB1*0201; DPB1*0301; DPB1*0401; DPB1*0402; DPB1*0501; DPB1*1301; DQA1*0101; DQA1*0102; DQA1*0103; DQA1*0104; DQA1*0201; DQA1*03; DQA1*0301; DQA1*0401; DQA1*05; DQA1*0501; DQB1*02; DQB1*0201; DQB1*0202; DQB1*0301; DQB1*0302; DQB1*0303; DQB1*0402; DQB1*0501; DQB1*0502; DQB1*06; DQB1*0601; DQB1*0602; DQB1*0603; DQB1*0604; DRB1*01; DRB1*0101; DRB1*03; DRB1*0301; DRB1*04; DRB1*0401; DRB1*0403; DRB1*0404; DRB1*0405; DRB1*07; DRB1*0701; DRB1*08; DRB1*0802; DRB1*080302; DRB1*09; DRB1*090102; DRB1*11; DRB1*1101; DRB1*110101; DRB1*1104; DRB1*12; DRB1*120201; DRB1*13; DRB1*1301; DRB1*1302; DRB1*14; DRB1*1401; DRB1*15; DRB1*1501; DRB1*150101; DRB1*1502; DRB1*16; DRB1*1601; and DRB1*160201. This group of MHC antigens is referred to hereinafter as “10/90 group of MHC antigens”.

Based on data from [45], the following MHC antigens were found to be present at an average frequency of from 15% to 85% as of the priority date of this application:

A*01; A*02; A*0201; A*03; A*11; A*1101; A*24; A*2402; B*07; B*0702; B*08; B*0801; B*13; B*1301; B*15; B*1525; B*35; B*39; B*3901; B*40; B*4001; B*4002; B*44; B*48; B*4801; Cw*01; Cw*0102; Cw*03; Cw*0304; Cw*030401; Cw*04; Cw*0401; Cw*0403; Cw*0501; Cw*06; Cw*0602; Cw*07; Cw*0701; Cw*0702; Cw*08; Cw*0801; DPB1*0201; DPB1*0301; DPB1*0401; DPB1*0402; DPB1*0501; DQA1*0101; DQA1*0102; DQA1*0103; DQA1*0201; DQA1*03; DQA1*0301; DQA1*0401; DQA1*05; DQA1*0501; DQB1*02; DQB1*0201; DQB1*0202; DQB1*0301; DQB1*0302; DQB1*0402; DQB1*0501; DQB1*06; DQB1*0601; DQB1*0602; DRB1*01; DRB1*03; DRB1*0301; DRB1*04; DRB1*07; DRB1*0701; DRB1*08; DRB1*0802; DRB1*080302; DRB1*09; DRB1*090102; DRB1*11; DRB1*110101; DRB1*12; DRB1*120201; DRB1*13; DRB1*14; DRB1*1401; DRB1*15; DRB1*1501; DRB1*150101; and DRB1*160201. This group of MHC antigens is referred to hereinafter as “15/85 group of MHC antigens”.

Based on data from [45], the following MHC antigens were found to be present at an average frequency of from 20% to 80%, as of the priority date of this application:

A*01; A*02; A*0201; A*03; A*11; A*1101; A*24; A*2402; B*1301; B*35; B*3901; B*40; B*4001; B*44; Cw*03; Cw*030401; Cw*04; Cw*0401; Cw*07; Cw*0701; Cw*0702; Cw*0801; DPB1*0201; DPB1*0401; DPB1*0402; DPB1*0501; DQA1*0102; DQA1*03; DQA1*0301; DQA1*05; DQA1*0501; DQB1*02; DQB1*0201; DQB1*0301; DQB1*0302; DQB1*06; DRB1*0301; DRB1*04; DRB1*07; DRB1*0701; DRB1*080302; DRB1*11; DRB1*110101; DRB1*12; DRB1*120201; DRB1*13; DRB1*14; and DRB1*15. This group of MHC antigens is referred to hereinafter as “20/80 group of MHC antigens”.

Based on data from [45], the following MHC antigens were found to be present at an average frequency of from 25% to 75%, as of the priority date of this application:

A*01; A*02; A*0201; A*11; A*1101; A*24; A*2402; B*1301; B*3901; B*4001; Cw*03; Cw*030401; Cw*07; Cw*0701; Cw*0702; DPB1*0201; DPB1*0401; DPB1*0402; DQA1*0102; DQA1*03; DQA1*0301; DQA1*05; DQA1*0501; DQB1*02; DQB1*0201; DQB1*0301; DQB1*06; DRB1*04; DRB1*080302; and DRB1*120201. This group of MHC antigens is referred to hereinafter as “25/75 group of MHC antigens”.

Based on data from [45], the following MHC antigens were found to be present at an average frequency of from 30% to 70%, as of the priority date of this application:

A*02; A*0201; A*11; A*1101; A*24; A*2402; Cw*03; Cw*030401; Cw*07; DPB1*0201; DPB1*0401; DQA1*0102; DQA1*03; DQA1*05; DQA1*0501; DQB1*02; DQB1*0301; and DQB1*06. This group of MHC antigens is referred to hereinafter as “30/70 group of MHC antigens”.

In one embodiment, all MHC antigens to be determined with the test kit of the invention are selected from one of the above groups of MHC antigens. By selecting MHC antigens according to the frequency in the population, a significant result can be obtained without having to analyse a vast number of possible MHC alleles on the level of the genome. The frequency of an MHC antigen is defined as the proportion with which a given antigen can be detected in a given population. A population can, for example, be a geographically defined group of individuals, for example human individuals. As used herein, the frequency of an MHC antigen is identical to the average phenotype frequency determined on the basis of data from [45] and/or http://www.allelefrequencies.net, as of the priority date of this application.

Suitable antigens include, but are not limited to, those listed in Tables 1 and 2. In a specific embodiment, the MHC antigens to be analyzed by the test kit of the invention are selected from the group consisting of HLA-A*01, HLA-A*02, HLA-A*03, HLA-A*11, HLA-A*24, HLA-A*26, HLA-A*30, HLA-A*31, HLA-A*68, HLA-B*07, HLA-B*08, HLA-B*15, HLA-B*35, HLA-B*40, HLA-B*44, HLA-B*51, HLA-Cw*01, HLA-Cw*03, HLA-Cw*04, HLA-Cw*07, HLA-DPB1*0201, HLA-DPB1*0301, HLA-DPB1*0401, HLA-DQA1*0101, HLA-DQA1*0102, HLA-DQA1*0201, HLA-DQA1*03, HLA-DQA1*0301, HLA-DQA1*05, HLA-DQA1*0501, HLA-DQA1*0505, HLA-DRB1*01, HLA-DRB1*03, HLA-DRB1*0301, HLA-DRB1*04, HLA-DRB1*07, HLA-DRB1*0701, HLA-DRB1*11, HLA-DRB1*13, HLA-DRB1*15, and HLA-DRB1*1501. In another embodiment, the set of MHC antigens to be analyzed comprises both class I and class II MHC antigens.

In order to determine whether or not an individual has a certain MHC antigen (i.e. whether or not he/she expresses it), the presence or absence of an analyte in a test sample from said individual is detected in accordance with this invention.

The term “test sample” refers to a material suspected of containing the analyte. The test sample may, for instance, include materials derived from a biological source, such as a physiological fluid, including, saliva, sweat, urine, blood, interstitial fluid, plasma, ocular lens fluid, cerebral spinal fluid, milk, ascites fluid, mucous, synovial fluid, peritoneal fluid, vaginal fluid, menses, amniotic fluid or the like. The body fluid may be diluted or processed prior to applying it in the test kit.

As used herein, the term “analyte” refers to the substance to be detected. The analyte which is suspected of being present in the test sample and which is to be detected may be any substance indicative of a specific MHC antigen.

MHC molecules, their fragments, degradation products of their peptide ligands and products of MHC-dependent microflora have all been considered as potential odorants. The most significant contacts between peptides and the MHC molecules are mediated through the side chains of so-called anchor residues of the peptide fitting into pockets of the MHC molecules. The range of peptides displayed by the MHC molecules of an individual mirrors the structural diversity of its MHC alleles. Different peptide ligands of one particular MHC molecule share common residues (so-called anchors) whose side chains fit into the characteristic binding pockets of the MHC molecule [25]. This suggests that anchor residues could be the defining feature of peptides and olfactory assessment might focus on them. MHC-peptide complexes are shed from the cell surface and their fragments appear in serum, saliva, sweat and urine [41]. The molecular weight for MHC membrane proteins and peptide ligands found in human saliva is in the range of 40 kD [12] and similar proteins have been detected in urine and sweat. Soluble HLA molecules between 45 kD (class I heavy chain) and 23 kD have been found in urine and 45 kD and 40 kD soluble HLA associated molecules in sweat.

Preferably, the analyte is selected from the group consisting of MHC molecules, fragments of MHC molecules, MHC ligands, fragments of MHC ligands, and combinations thereof. For example, the analyte may be a peptide ligand which can be bound by its respective MHC molecule in the molecular groove. Alternatively, the peptide may already have been partially degraded resulting in fragments which may be detected as well. In body fluids such as saliva and urine, full length MHC molecules may be present. Usually, however, fragments of MHC molecules such as the extracellular portion thereof or fragments thereof are present in the test sample. It is also possible to detect a combination of MHC molecule and its ligand bound thereto. For instance, analytes may be MHC (major histocompatibility) components. These compounds may be part of the MHC gene complex or correlated to it and may include MHC-molecules, MHC-ligands, MHC-correlated peptides, MHC-correlated odour components, MHC-derived odour components, MHC-correlated carboxylic acids or antibodies to any of the above substances. In a particular embodiment, the analyte is selected from the group consisting of MHC molecules, fragments of MHC molecules, naturally occurring derivatives of MHC molecules, MHC ligands, fragments of MHC ligands, naturally occurring derivatives of MHC ligands and combinations thereof.

The test kit may be any kit or device suitable for rapidly determining an MHC profile of an individual. The analyte can be detected in a “sandwich” or in a competition assay. The general principle of such test formats is known to the skilled person. Particularly preferred are lateral flow assays as described in e.g. EP 291 194 B1, EP 186 799 B1, or WO 2005/121800 A2. The general principles of the test kits, test devices and assay methods disclosed in U.S. Pat. No. 4,956,302; U.S. Pat. No. 6,372,513; U.S. Pat. No. 5,770,460 and/or U.S. Pat. No. 4,943,522 can be applied in the present invention.

### Sandwich Format

The so-called sandwich format of the test kit is preferred when the analyte is an MHC receptor, a soluble form thereof or a fragment thereof.

This assay format typically involves a porous media having a mobilizable labeled antibody and an immobilized unlabeled antibody partner for the analyte of interest in the biological sample. These antibodies are often referred to as the conjugate antibody and the capture antibody. The sample is added to the porous media, to allow for formation of labeled mobilizable product which moves along the porous media to contact and react with the capture antibody to form a fixed, detectable, concentrated capture antibody/analyte/detection reagent complex. Sandwich assays may include immunological assays wherein the labeled reagent and the second binding partner are both antibodies, or are both antigens, and may also include other types of molecules. For example, enzyme immunoassays (EIA) and enzyme-linked immunosorbent assays (ELISA) are types of sandwich immunoassays, in which the binding is between an antibody and an antigen, and the labeling partner is an enzyme.

Typically, these chromatographic assay devices are comprised of a porous chromatographic medium which acts as the matrix for the binding assay. The sample of interest is added directly or indirectly to one end of the medium, and is chromatographically transported to a detection reagent with which it reacts to form a labeled conjugate product, which is then transported to a test zone containing an immobilized capture reagent such as a capture antibody, in which the presence, absence, or quantity of an analyte of interest can be determined.

The preferred test kit of the invention comprises a plurality of different pairs of binding reagents; usually it comprises N mobilizable labelled primary binding reagents and N immobilizable unlabelled secondary binding reagents, wherein N is an integer as defined hereinabove. The binding reagent may be any molecule capable of binding to the analyte. Preferably, the binding reagent is an antibody, e.g. a monoclonal antibody or a polyclonal antibody. As used herein, the term “antibody” is meant to include intact molecules as well as antibody fragments (such as, for example, Fab and F(ab′)₂ fragments) which are capable of specifically binding to the analyte. Fab and F(ab′)₂ fragments lack the F_{c} fragment of intact antibody. Moreover, antibodies in accordance with the present invention include single chain antibodies. Other binding reagents such as aptamers may also be envisaged as long as they are capable of specifically binding to the analyte.

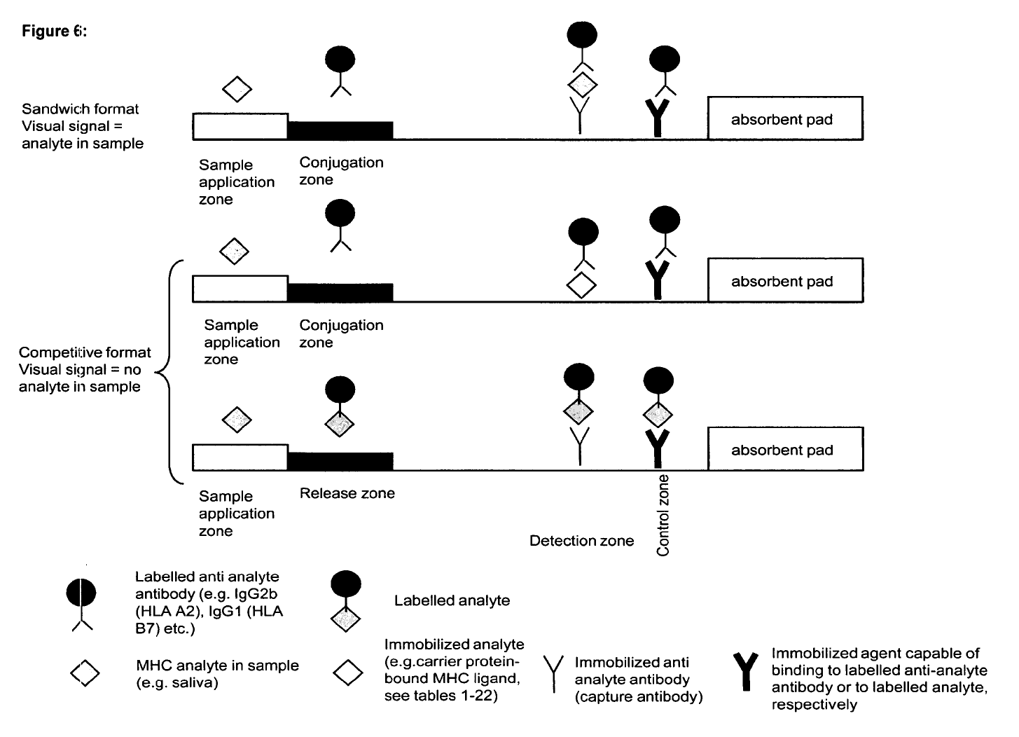

MHC class I antigen-antibody conjugates have been produced for rats (A^{c} with YR5/12 and A^{av1} with JY1/116) [41] and humans (class I associated proteins with 4B5.1 monoclonal primary antibody and IgG phosphatase-conjugated goat anti-mouse or anti-rabbit secondary antibody) [29]. Suitable antibodies specifically recognizing the analyte can be generated or otherwise obtained by the skilled person. Suitable techniques for generating antibodies are disclosed in, e.g., Harlow and Lane, “Antibodies, A Laboratory Manual” CSH Press 1988, Cold Spring Harbor N.Y. Specifically, the following examples for commercially available antibodies for the sandwich type assay shall visualize the manufacturing of such a test kit (See FIG. 6): Control antibodies can be labeled rat anti mouse IgG1 heavy chain:HRP (AbD Serotec No. MCA336P) or mouse anti human beta 2 microglobulin (AbD Serotec No. MCA 1116) labeled with Lynx rapid HRP antibody conjugation Kit (AbD Serotec No. LNK002P). Detection (capture) antibodies can for example be mouse anti human HLA A2 (AbD Serotec No. MCA2090), mouse anti human HLA A24 (antikoerper-online.de, No. ABIN131788), mouse anti human HLA B7 (AbD Serotec No. MCA986), mouse anti human HLA DQw1 (antikoerper-online.de, No. ABIN100554) mouse anti human HLA DRA (antikoerper-online.de, No. ABIN228532), mouse anti human HLA DR (AbD Serotec No. MCA1879) etc.

Thus, for each MHC-antigen indicating analyte, two binding reagents specifically recognizing the analyte are included. This allows a more sensitive detection of the analyte. A given pair of binding reagents consists of (i) an unlabelled secondary binding reagent that is capable of specifically binding to an analyte that is indicative of a predefined MHC antigen, and (ii) a labelled primary binding reagent that is capable of specifically binding to the same analyte. In the case of antibodies as binding reagents, the two antibodies of a pair of antibodies preferably recognize different epitopes present in the analyte molecule(s). Each pair of binding reagents contained in the kit usually detects an MHC antigen different from the MHC antigens detected by the other pairs of binding reagents in the same kit. Accordingly, a test kit containing N pairs of binding reagents will allow detection of N different MHC antigens, respectively.

With reference to the frequency-based selection of MHC antigens to be analysed by the test kit of this invention, the test kit preferably comprises N different pairs of binding reagents capable of specifically recognizing N different MHC antigens, respectively, wherein said N different MHC antigens are selected from the 10/90 group of MHC antigens.

More preferably, the test kit comprises N different pairs of binding reagents capable of specifically recognizing N different MHC antigens, respectively, wherein said N different MHC antigens are selected from the 15/85 group of MHC antigens.

Still more preferably, the test kit comprises N different pairs of binding reagents capable of specifically recognizing N different MHC antigens, respectively, wherein said N different MHC antigens are selected from the 20/80 group of MHC antigens.

Even more preferably, the test kit comprises N different pairs of binding reagents capable of specifically recognizing N different MHC antigens, respectively, wherein said N different MHC antigens are selected from the 25/75 group of MHC antigens or from the 30/70 group of antigens.

### Competitive Format

The so-called competitive format of the test kit is preferred when the analyte is an MHC ligand, a fragment or a derivative thereof.

In a first embodiment, the test kit in the competitive format comprises: at least one solid substrate in contact with a proximal sample application zone, said substrate having N conjugation zones and N distal detection zones, said conjugation zones respectively containing N labelled primary binding reagents capable of binding with an analyte to form an analyte-primary binding reagent conjugate; said distal detection zone respectively having immobilized thereunto, N unlabeled secondary binding reagents capable of binding to the respective labelled primary binding reagent, but not to a migrating analyte-labeled primary binding reagent conjugate to determine the presence or absence of N analytes which are indicative of defined N-MHC antigens, wherein N is an integer greater than 3.

In this first embodiment, the labelled primary binding reagent is preferably a labelled antibody specifically recognizing the analyte. The secondary binding reagent is preferably an immobilized analyte which is in the detection zone. The labelled antibody can bind to the immobilized analyte, while the labelled antibody cannot bind to the immobilized analyte when it is bound to the analyte in an analyte-antibody complex. Thus, the migrating analyte competes with the immobilized analyte for binding to the labelled antibody. The lack of a signal in the detection zone is indicative of the presence of a given analyte in the sample.

In a second embodiment, the test kit in the competitive format comprises:

at least one solid substrate in contact with a proximal sample application zone, said substrate having N release zones and N distal detection zones, said release zones respectively containing N different labelled analytes; said distal detection zone respectively having immobilized thereunto, N unlabeled binding reagents capable of binding to a migrating labelled or unlabelled analyte to determine the presence or absence of N analytes which are indicative of defined N-MHC antigens, wherein N is an integer greater than 3.

In this second embodiment, the unlabelled binding reagent immobilized in the detection zone is preferably an immobilized antibody capable of specifically recognizing the analyte. The release zone preferably contains labelled analyte molecules which can migrate when sample liquid migrates from the sample application zone through the release zone into the direction of the detection zone. The unlabelled analyte from the sample then competes with the labelled analyte from the release zone for binding to the immobilized unlabelled binding reagents. The lack of a signal in the detection zone is indicative of the presence of a given analyte in the sample.

Tables 1 and 2 respectively show selection of MHC class I and class II alleles with corresponding peptide ligands to obtain the desired result for individual MHC profiles from lateral flow assay. Bold letters within selected ligands mark anchor residues likely to bind to groves of the corresponding MHC membrane protein.

The test kit of the instant comprises one or more solid substrates. A preferred substrate of the present invention is a porous medium, strip or membrane. In one embodiment, the test kit comprises one solid substrate which in turn comprises said at least 3 different pairs of binding molecules. In another embodiment, the test kit comprises 2 or more solid substrates, e.g. at least 3 solid substrates, 8 to 33 solid substrates or 8 to 12 solid substrates. In a specific embodiment, the number of solid substrates in the test kit is identical to the number of pairs of binding reagents comprised in the test kit, i.e. the test kit comprises N solid substrates. Suitable solid substrate materials include, but are not limited to, cellulose, nitrocellulose, polyethylene, polyvinyl chloride, polyvinyl acetate, copolymers of vinyl acetate and vinyl chloride, polyamide, polycarbonate, polystyrene; and “bibulous” materials such as those disclosed in U.S. Pat. No. 5,770,460 treated with blocking agents, e.g. with detergents or proteins. Suitable solid substrate materials are known to one of ordinary skill in the field.

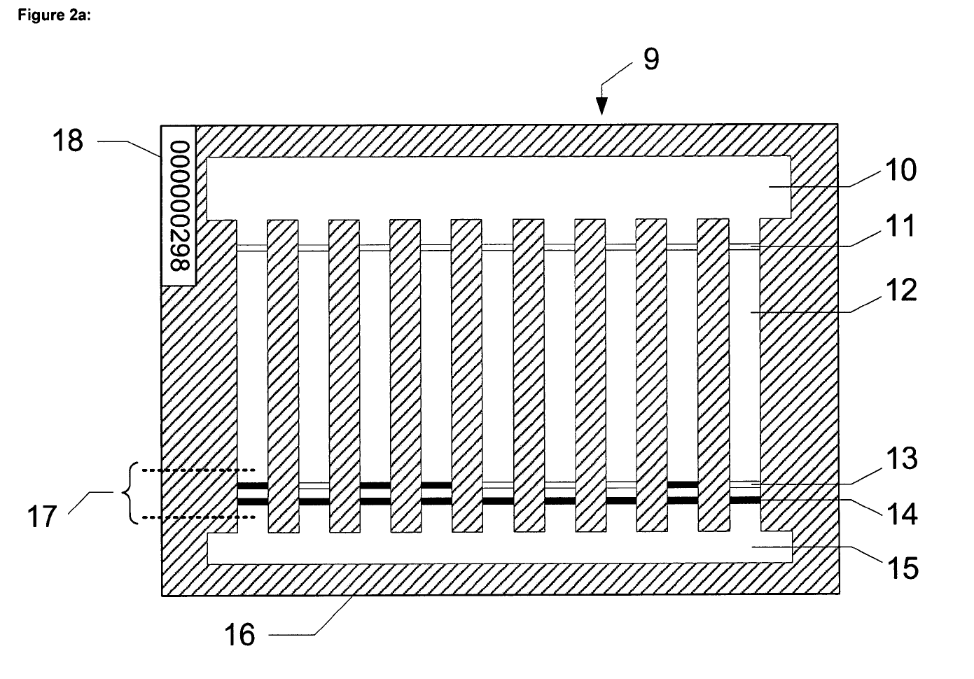

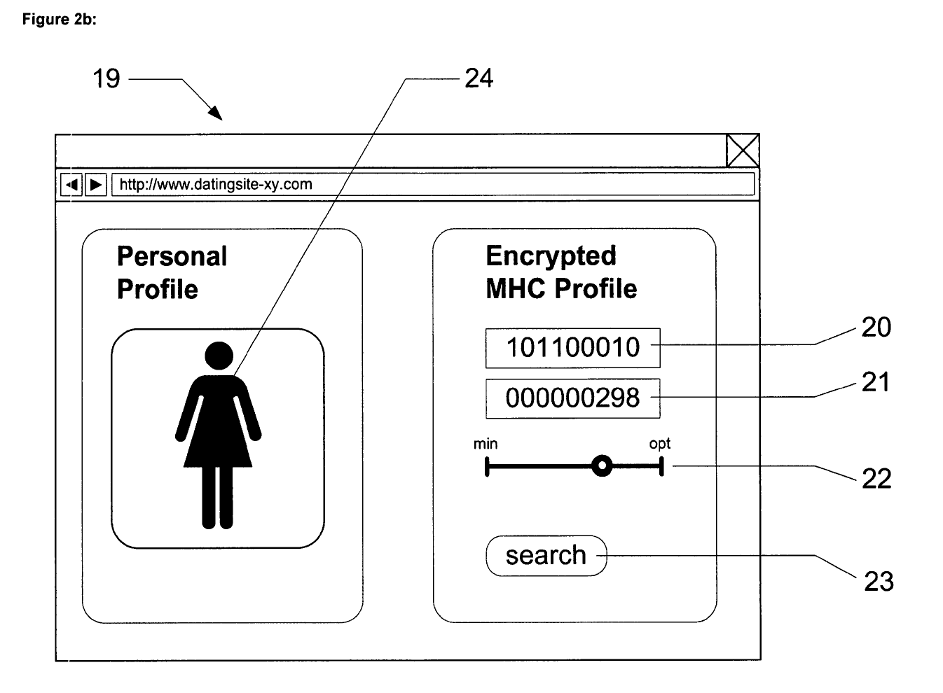

Referring to FIG. 2a, the solid substrate may be a lateral flow membrane, which has on one end, a proximal sample application zone to receive a liquid sample, and further upstream, a conjugation zone wherein the analyte conjugates with a mobilizable labeled primary binding reagent, and further upstream, at least one distal detection zone having an immobilized unlabelled binding reagent affixed thereunto. The test sample contains an analyte which is an analyte which can be derivatized so as to bind the immobilized member. In one preferred configuration, the membrane is bound to two substantially fluid-impervious sheets, one on either side, with openings on one side or both sides to provide definition to the application and detection zones.

The lateral flow achieved in the kit and method of the invention is the result of the properties of the lateral flow membrane. The membrane has a much smaller thickness than surface dimension and may be hydrophilic enough to be wetted and thus permit aqueous solutions and materials to exhibit lateral flow freely, and preferably isotropically, at substantially the same rates for various components of a sample.

The membrane usually includes more than one detection zone (see FIG. 2a and Example 1), usually it includes N detection zones, along with control and reference zones. Multiple detection zones are designed to detect different analytes indicative of different MHC antigens. Multiple detection zones may be in any spatial relationship to the application zone, since the membrane itself does usually not provide a barrier to sample flow.

In the method of the invention, the sample, to be analyzed for MHC-antigen indicating analytes is applied to the proximal sample application zone and allowed to be transported laterally through the membrane via a conjugation zone containing the first member of the binding reagent pair to a distal detection zone, where there is, immobilized on the membrane, the other, second member of the binding reagent pair.

In the detection zone, mobilizable binding reagent-analyte conjugate binds to the immobilized binding reagent, and the resulting bound complex is detected. Detection may use any of a variety of labels and/or markers, e.g., enzymes, liposomes, fluorescent tags, polymer dyes, or colored particles, etc., and detection is by means of, for example, direct visual observation, by developing a color, by fluorescence measurement, or by any of many other techniques by which the presence or absence of a chemical or biochemical species may be detected directly or indirectly.

The kit may be encased in a housing. The housing can be fabricated out of any convenient material (e.g. HDPE, LDPE, PP, polystyrene, acrylic, polycarbonate, etc.).

The rapid assay to which a test sample is applied is designed such that it can measure a set of different analytes that are present within one test sample. The number of different analytes may be N as defined hereinabove, preferably it is 8-12. Each detected analyte contributes to the assay result.

It is preferred that the test kit of the invention comprises an item or article on which a unique identifier is recorded. The unique identifier may be any information suitable to uniquely characterize the test kit. The unique identifier may be a number such as a serial number consisting of several digits (for example 5, 6, 7, 8, 9, 10, 11 or 12 digits), a barcode or a graphic representation which can be identified by a scanner.

The item on which the unique identifier is recorded may be any article, product or the like suitable to carry such information. It is preferred that the unique identifier is imprinted on said item or article. Preferably, the item or article is made of paper or cardboard. In another preferred embodiment, the unique identifier is imprinted on the solid substrate of the test kit. In yet another preferred embodiment, the unique identifier is imprinted on a housing in which the solid substrate is encased. To obtain an encrypted MHC profile, the configuration of the sensors, the signals or the substrates in the rapid assay may be encrypted. The key, which is related to said unique identifier, together with the encrypted MHC profile yields a decrypted MHC profile. The unique identifier may be a serial number or a random number without replacement or any other unambiguous number. This number may be indicated on the rapid assay.

### Method of Matching Individuals

One embodiment of the present invention comprises the steps of obtaining a plurality of personal profiles from a plurality of individuals and storing said personal profiles in a database on a computer. The personal profile may encompass contact data such as postal address, telephone number, e-mail address of the individual. The personal profile may further include a photograph or image of the individual as well as personal attributes and preferences (e.g. height, colour, gender, weight, activities and the like). The personal profiles may be entered by the individual into a form on a computer screen provided by a first service provider, e.g. an internet dating service. There may be several fields which must be filled and further fields which may optionally be filled. By confirming the entry of his/her personal profile by clicking on a virtual button on the computer screen, the personal profile is transmitted to a first service provider which may be the dating service. Once the personal profile is received by the service provider, it is stored in a database on a computer. Known forms of suitable databases are known of ordinary skill in the field.

Alternatively, the personal profile may be transmitted by e-mail, by normal mail, via telefax or over the telephone.

In a further step, a test kit for rapidly determining an individual's MHC profile is provided to those individuals who have already provided their personal profiles. Alternatively, the test kit may be offered to interested individuals who have not yet provided their personal profiles. If interested, they may use the kit, determine their encrypted MHC profile and enter the results together with their personal profiles into the platform of the dating service, or enter the encrypted MHC profile in the personal data separately.

Preferably, the test kit is a test kit according to the present invention as described herein above.

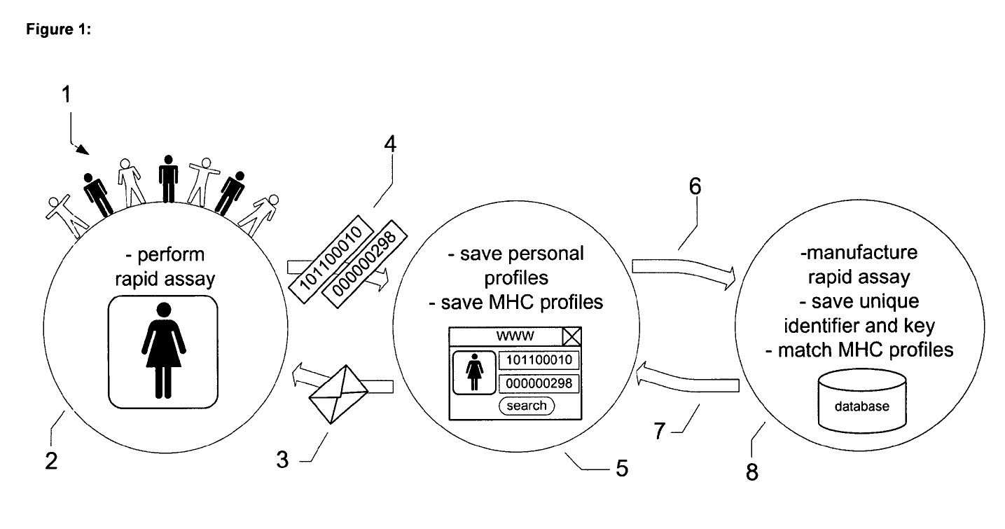

Referring to FIG. 1, one embodiment of the invention can be summarized as follows: A user from a defined community 2 receives from a dating service a rapid assay 3 to detect an encrypted MHC profile. Said user performs the rapid assay and adds the results of the rapid assay (the encrypted MHC profile) on an interface of the dating service 4 to the personal profile of said user together with a unique identifier indicated on the rapid assay. The dating service 5 sends the results and the unique identifier 6 to a matching service 8. The matching service converts the assay results using a key, which is related to said unique identifier, into a decrypted MHC profile. The pair wise distance between decrypted MHC profiles is then converted into a quality of match, where intermediate distances between decrypted MHC profiles constitute better matches than large or small distances. The quality of match is sent to the dating service 7 from where said user can acquire the quality of match between said user and any other user with an account at the dating service and a valid entry for the assay result and unique identifier. It should be understood, that the company shipping the rapid assay to the user is preferably different from the matching service but can be identical to the dating service or to any other service. The company shipping the rapid assay to the user may even be the manufacturer.

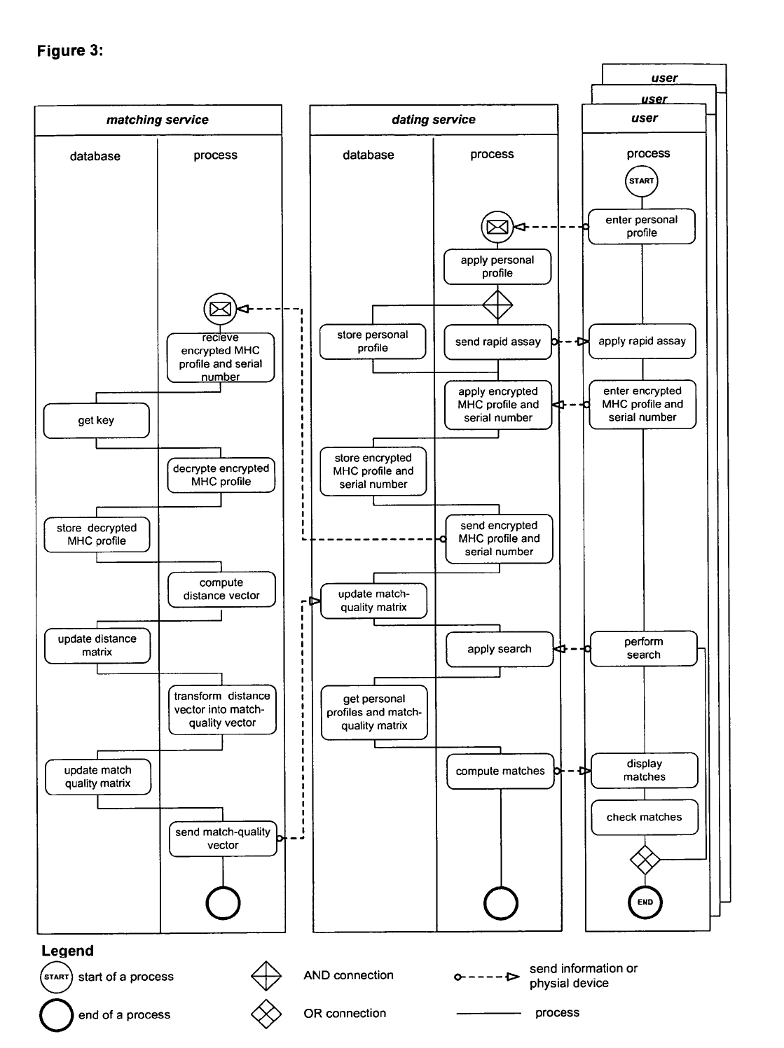

FIG. 3 is a process chart of matching and dating services according to one embodiment of the present invention showing in chronological order, the preferred steps usable to provide a user with a list of potential dating partners that match said user's MHC profile.

Together with the encrypted MHC profile, the respective unique identifier is transmitted from the user to the first service provider. Accordingly, the entry form on the computer screen provided by the dating service is preferably adapted to allow the entry of two pieces of information, namely (1) the encrypted MHC profile and (2) the unique identifier. Preferably, the filling of both fields is mandatory. Entry of only one of both pieces of information would not allow transmission of the data.

The encrypted MHC profiles and the respective unique identifiers are stored in a database, preferably in a database on a computer. Each encrypted MHC profile and its corresponding unique identifier is allocated to one of the personal profiles.

The encrypted MHC profiles are decrypted using the decryption key, which is related to said unique identifier, preferably by the aid of a computer. The thus decrypted MHC profiles are than stored in a database, preferably in a database on a computer.

The matching of the decrypted MHC profiles involves two steps. Namely the step of calculating a distance measure and the transformation of said distance measure into a quality of match. The distance measure is a metric or distance function which defines a distance between elements of a set. Here we refer to the mathematical definition. Specifically a metric on a set X is a function d: X×X→R (where R is the set of real numbers). For all x, y, z in X, this function is required to satisfy the following conditions:

d(x,y)≧0 1.

d(x,y)=0 if and only if x=y 2.

d(x,y)=d(y,x) 3.

d(x,z)≦d(x,y)+d(y,z) 4.

The distance measure may for instance be a Euclidian distance, a Mahalanoby distance or any other metric suitable for the MHC resolution addressed by the detected analytes. The distance measure may include the phylogeny of the MHC genes coding for the detected analytes such as the measures for example developed by or based on the following authors [42-44].

The next step thus comprises calculating the pairwise distances in decrypted MHC profiles between all individuals for which decrypted MHC profiles have been stored in the database.

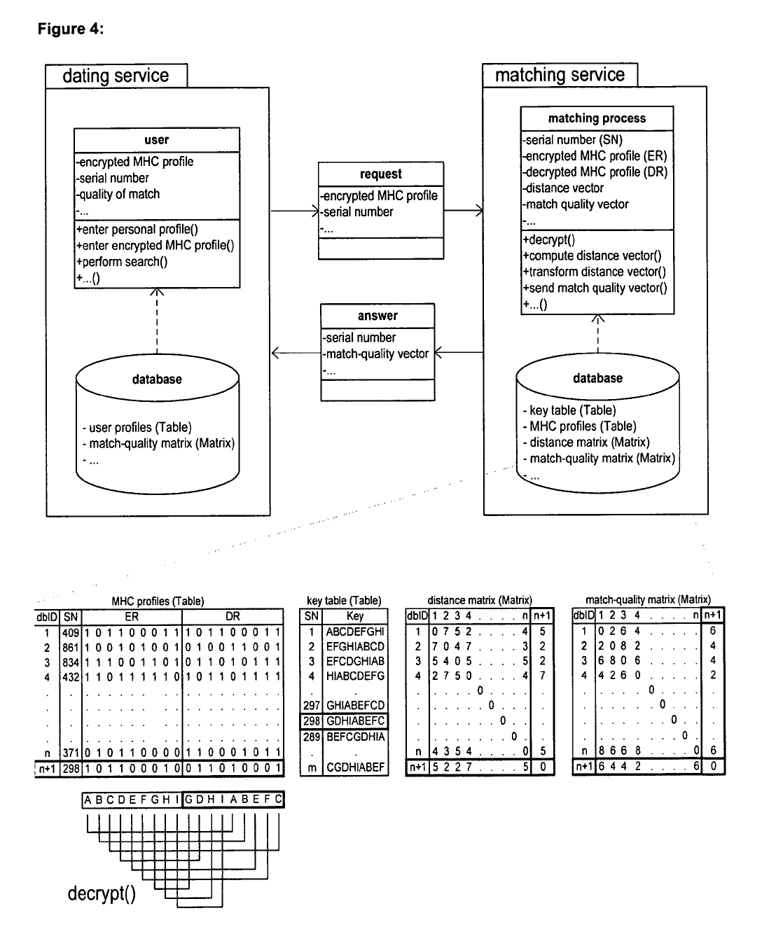

Theses results can be used to generate a matrix that contains the quality of match for each pairwise distance as depicted in FIG. 4. From the pairwise distances, the quality of match between individuals can be calculated, preferably using a computer.

The quality of match may be defined as being “high” or “low”. The quality of match between two individuals can be defined as “high” if more than 10% and less than 90% of the MHC antigens in the MHC profiles of the two individuals are the same. Preferably, the quality of match is regarded as “high” if more than 15% and less than 85% of the MHC antigens in the MHC profiles of the two individuals are the same. More preferably, the quality of match is regarded as “high” if more than 25% and less than 75% of the MHC antigens in the MHC profiles of the two individuals are the same.

The method of the invention may further comprise the step of providing to a first individual contact data of further individuals whose MHC profiles have a high quality of match with the MHC profile of said first individual. These data are usually provided only upon request by the first individual. The request may be submitted “online” by clicking on a virtual button or link on the computer screen.

In a specific embodiment, steps (a), (b), (c) and (d) are carried out by a first service provider while steps (e), (f) and (g) are carried out by a second service provider. That is, the decryption of the MHC profile is carried out by a different entity than that having contact with the user. For example, steps (e), (f) and (g) may be carried out by a so called matching service whereas steps (a) through (d) are carried out by the dating service which offers to provide a match between individuals.

### Method for Preparing Customized Odour Compositions

The same MHC profiles can also be used to manufacture customized odours for a given user. The method comprises (a) providing or determining an MHC profile of one or more individuals. It is preferred that the MHC profile is not determined on the nucleic acid level (e.g. by genotyping etc.). The number of individuals is not particularly limited, it may be 1 or more, e.g. at least 10 or at least 100, for example 10 to 10000 or 100 to 1000. Usually, one customized odour composition is prepared for each individual for which an MHC profile has been provided or determined. The determination of the MHC profile can be carried out as described hereinabove, e.g. using the test kit described herein. Other kits and techniques can be employed as well. The MHC profile contains information on the presence (expression) or absence (no expression) of N different MHC antigens in the individual. N has the same meaning as defined above with respect to the test kit of this invention. The MHC antigens are as defined above with respect to the test kit of this invention.

For each individual, at least one peptide ligand of an MHC antigen which in step (a) is found to be expressed in the individual is selected. That is, if the individual is positive for a certain WIC antigen, the peptide ligand of this MHC antigen is identified as described herein (preferably using the program available at www.syfpeithi.de). After having identified it, the peptide ligand is selected according to the method of this invention.

Alternatively, or additionally, at least one peptide ligand of an MHC antigen which in step (a) is found not to be expressed in the individual is selected. That is, if the individual is negative for a certain MHC antigen, the peptide ligand of this MHC antigen is identified as described herein (preferably using the program available at www.syfpeithi.de). After having identified it, the peptide ligand is selected according to the method of this invention.

Step (b) may comprise selecting one, two, three, four, five or more different peptides.

The selected peptide(s) may have a length of 5 to 50, preferably of 6 to 40, more preferably of 7 to 30, more preferably of 8 to 20, most preferably of 8 to 15 or 8 to 12 amino acids.

The selected peptide(s) may be ligands of an MHC antigen selected from one of the above groups of MHC antigens. In a first embodiment, the selected peptide(s) is (are) ligands of an MHC antigen selected from the “5/95 group of MHC antigens”. In a second embodiment, the selected peptide(s) is (are) ligands of an MHC antigen selected from the “10/90 group of MHC antigens”. In a third embodiment, the selected peptide(s) is (are) ligands of an MHC antigen selected from the “15/85 group of MHC antigens”. In a fourth embodiment, the selected peptide(s) is (are) ligands of an MHC antigen selected from the “20/80 group of MHC antigens”. In a fifth embodiment, the selected peptide(s) is (are) ligands of an MHC antigen selected from the “25/75 group of MHC antigens”. In a sixth embodiment, the selected peptide(s) is (are) ligands of an MHC antigen selected from the “30/70 group of MHC antigens”.

The selected peptide(s) may have a sequence as shown in any one of SEQ ID NO:1 through 626.

For each individual, a compound is provided which comprises (1) the selected peptide ligand, (2) a fragment of the selected peptide ligand, or (3) a derivative of the selected peptide ligand. The compound preferably is a peptide. It may, however, also be a compound other than a pure peptide consisting of amino acids, e.g. it may be a peptide coupled to non-amino acid structures. The fragment of the peptide comprised in the compound preferably consists of at least 5, more preferably at least 7, still more preferably at least 10, most preferably at least 12 consecutive amino acids of the amino acid sequence of the selected peptide. The fragment of the peptide comprised in the compound may consist of 5 to 12, preferably of 6 to 11, more preferably of 7 to 10, most preferably of 8 or 9 consecutive amino acids of selected peptide. The compound may comprise 1 or 2 or more (e.g. 3, 4, 5, 6, 7, 8, 9 or 10) foreign amino acids not present in the selected peptide. These foreign amino acids may be fused to the N-terminus or to the C-terminus of said fragment of the selected peptide. The derivative of the selected peptide may be an acylated, (e.g. acetylated), amidated, esterified form of the selected peptide or any other derivative including, but not limited to, oxidized forms, reduced forms, peptides coupled to a carrier or linker etc. Preferred embodiments of the derivatives are peptides, in which 1 to 10 (e.g., 1, 2, 3, 4, 5, 6, 7, 8, 9 or 10) amino acid residues of the selected peptide are substituted, added or deleted, in any combination. Particularly preferred are substitutions, additions, and deletions that do not alter the properties or activities of the selected peptide. The substitutions may be conservative amino acid substitutions, whereby a residue is substituted by another with like characteristics. Typical conservative substitutions are among Ala, Val, Leu and Ile; among Ser and Thr; among the acidic residues Asp and Glu; among Asn and Gln; and among the basic residues Lys and Arg; or aromatic residues Phe and Tyr. Preferred embodiments are peptide derivatives that retain substantially the same biological function or activity as the selected peptide.

Three properties can increase the storage life and the adaptability of such custom odour stock solutions:
1) The chosen peptides is preferably smaller than 20-mers.
2) Peptides containing Cystein or Methionin tend to oxidate and may be avoided
3) There may be constraints concerning the synthesis of the peptides e.g. distribution of amino acids with positive charge (K, R, H) etc.

Customization can be classified into “enhance” and “complement”. A custom odour for “enhance” is a mixture of any number of the MHC peptides corresponding to the detected analytes on the rapid assay for said user. A custom odour for “complement” comprises MHC peptides matching the detected analytes of said user with a desired degree of matching quality as defined hereinabove or in example 3. For instance, matching could denote peptides corresponding to MHC profiles with a quality of match of 70 percent to the profile of said user. The concept of “enhance” and “complement” is explained in more detail in the example 3, with reference to the metrics and the MHC profiles used in this application.

When a “enhance” odour composition is to be provided, the method comprises selecting peptide ligands of a least 90%, preferably of at least 95%, most preferably at all of the MHC antigens found to be expressed in said individual. Similarly, not more than 10%, preferably less than 10%, more preferably less than 5%, most preferably none of the MHC antigens found not to be expressed by said individual should be selected.

When an odour composition of the type “complement” is to be provided, the method may comprise selecting peptide ligands of 25 to 75% of all MHC antigens found to be expressed in said individual, and selecting 25 to 75% of all MHC antigens found not to be expressed in said individual.

Customized odours can contain single peptides or mixtures of the peptides mentioned above. Customized odours can be the odorant itself or they can be used as stock solutions to supplement existing fragrances. Customized odours can be added to existing fragrances, cosmetic products and even food items and beverages. For instance matching could denote peptides corresponding to MHC profiles with a quality of match of 70 percent of said user. The customized odour can contain one single peptide or any number of peptides in any combination matching said user.

Preferably, “providing the compound” includes “preparing the compound”, more preferably it includes “synthesizing the compound”. This can be done by solid phase peptide synthesis according to techniques known in the art. The corresponding peptides may be synthesized by standard procedures in a purity of at least 70%, preferably at least 75% more preferably at least 80%. Preferably the peptide is dissolved in water. Some peptides may require adding NH₄OH, urea, guanidine-HCL in deionised water depending on the overall charge of the peptide. Handling and storage of peptides is following standard procedure (described for example in the SIGMA-ALDRICH Guidelines 2007).

The invention further relates to an odour composition obtainable as described herein. This composition may comprise 1, 2, 3 or more different peptides. The different peptides are usually ligands of different MHC antigens. The length of the peptides is at least 7 amino acids, preferably 8 to 30, more preferably 10 to 25, more preferably 12 to 20, most preferably 15 to 19 amino acids.

The peptides comprised in the compositions may be the peptides selected in the method of the invention, as described hereinabove. In one embodiment, the composition comprises one, two, three, four or five different peptides selected from the group consisting of SEQ ID NO:1 through 626. In another embodiment, all peptides comprised in the composition are selected from the group consisting of SEQ ID NO:1 through 626.

The concentration of the peptides in the composition may range from 1*10⁻³ to 1 gL⁻¹, preferably from 0.01 to 0.8 gL⁻¹, most preferably from 0.1 to 0.3 gL⁻¹. The odour compositions may be used to enhance one's own body odour (“enhance”).

A preferred embodiment of the invention relates to the fields of cosmetics, particularly the manufacture of a perfume. The bodily odour can be influenced in a predetermined manner by means of adding the compositions of the invention to perfumes. Cosmetic compositions according to the invention contain common cosmetic components, such as further active substances, carriers, diluents and/or adjuvants, apart from the MHC peptide ligands. For example the cosmetic compositions may be formulated as liquids, gels, creams, slurries, ointments, aerosols, sprays, suspensions, powders etc. together with other ingredients, e.g. further odorants, auxiliaries, carriers and/or diluents. The compositions may be administered topically, orally or nasally or by any other suitable means. Preferably, the compositions are applied to skin and/or clothes of an individual in a manner that allows access of the compound to the environment, e.g. air space, enclosing the individual. The concentration of the active substance in the cosmetic compositions preferably is preferably in the subnano to micromolar range and more preferably within the range of from 1 pM to 100 μM, preferably from 10 pM to 10 μM, more preferably from 0.1 nM to 10 μM, most preferably from 1 nM to 1 μM.

The following examples further illustrate the invention. The invention, however, is not limited to these exemplary embodiments.

### EXAMPLES

### Example 1

### Predicting the Matching Quality Between Users Based on an MHC Flow-Through Assay

In a first step, the user enters the personal profile into an online form. The personal profile comprises among other things the postal address of said user. Referring to FIG. 2b, for instance, it may also comprise a personal photograph 24. The personal profile of said user is then sent to the dating service. The dating service stores the personal profile into its database and sends a rapid assay kit to the postal address of said user.

A user performs the rapid assay. Referring to FIG. 2a, for instance, one embodiment of a rapid assay in our invention may be a flow-through assay device 9. To initiate the flow through assay, a user directly applies approximately 5 ml of saliva sample to a sample pad 10 through which it will travel in the direction of the wicking pad 15. In the conjugate zone 11, a gold colloid labelled primary antibody will bind to the analyte. The superfluous conjugate will travel further through the porous membrane made of nitrocellulose 12 to the detection region 13, where a secondary antibody binds to the conjugate. The primary and the secondary antibodies are designed and manufactured such that they detect MHC antigens with two epitopes. The sensitivity of the antibodies to detect MHC antigens in the saliva sample is approximately 50 ng ml⁻¹. Superfluous conjugate moves further to the control region 14 where an anti-species antibody binds to the conjugate. The rest of the conjugate will be absorbed by the wicking pad 15, which promotes capillary action and fluid flow through the porous membrane 12. Staining in the detection region 13 and the control region 14 together indicate the presence or absence of each of the nine analytes for which the flow through device has been designed. A serial number 18 is indicated on the rigid material 16 containing the device 9. Presence and absence of each of the nine analytes together constitute an encrypted MHC profile of the user.

Referring to FIG. 2b, the user enters the encrypted (ER) MHC profile 20 and the serial number 21 into the online form and sends it to the dating service 23. The dating service stores the encrypted MHC profile and the serial number of said user and sends it to the matching service.

The matching service receives the encrypted MHC profile and the serial number of said user. In order to decrypt the encrypted MHC profile of said user, the key to the corresponding serial number is sorted out from the key table in FIG. 4 and applied to the encrypted MHC profile of said user, which yields the decrypted (DR) MHC profile of said user. Then the decrypted MHC profile of said user is stored in the MHC profiles table (see FIG. 4). In the next step, the distance-vector of said user to the decrypted MHC profiles of all other users is computed and the distance matrix in FIG. 4 is updated with this distance vector.

The distance vector of said user is then transformed into a match-quality vector of said user. To compute the match-quality vector of said user, a function is applied to transform the distances of the distance-vector to match-quality parameters between 0 and 1 where 0 represents the lowest and 1 the best match-quality. The matrix that contains the quality of match is then updated with the vector that contains the quality of match of said user to all other users. Then the matching service sends the match-quality vector of said user to the dating service in order to protect said user's privacy, by separating said user's personal profile from said user's decrypted WIC profile. The dating service receives the vector of said user and updates its matrix.

Referring to FIG. 2b, in the next step said user enters the desired matching quality 22 and other preferences into the online form and sends the search criteria to the matching service 23. The search criteria are then processed and the necessary information is retrieved from the database of the matching service. Then the matches are computed and send to said user where they are displayed. Said user checks the matches and then either ends the search for a dating partner or performs a new search.

By way of example two possible ways how to calculate the “match-quality matrix” on the basis of the “MHC profiles” via the “distance matrix” are indicated in the following:

$${Dr}_{i} = \{ {r}_{i , 1} , {r}_{i , 1} , … {r}_{i , N} \}$$

$${d}_{i , j} = {\sum }_{k = 1}^{N} \vert {r}_{i , k} - {r}_{j , k} \vert$$

### Triangle-Curve (See Example 1)

$${q}_{i , j} = \{ \begin{matrix}\frac{1}{N} 2 {d}_{i , j} & 0 \leq {d}_{i , j} \leq \frac{N}{2} \\ \frac{1}{N} ( 2 n - 2 {d}_{i , j} ) & \frac{N}{2} < {d}_{i , j} \leq N\end{matrix}$$

### Quadratic-Curve

$${q}_{i , j} = \frac{4}{{N}^{2}} {d}_{i , j} ( N - {d}_{i , j} ) , 0 \leq {d}_{i , j} \leq N$$

r_{i,k} are single (boolean) assay results which together constitute the decrypted MHC profile DR
d_{i,j} is the distance function between the decrypted MHC profiles DRᵢ and DRⱼ
q_{i,j} is the function to obtain the quality of match to be entered in the match quality matrix.

### Example 2

### Selection of Target Antigens

Single MHC loci can have up to approximately 300 different alleles and most of these alleles occur in small frequencies in the population. Preferably, lateral flow assay of the instant invention has several test fields (9 in Example 1) of which each has a certain probability for a positive or negative result. If the frequency of the alleles is much higher or much lower than 0.5 in the population, then the expected diversity from individual MHC profiles will not be sufficient to effectively match the profile of two or several users with each other.

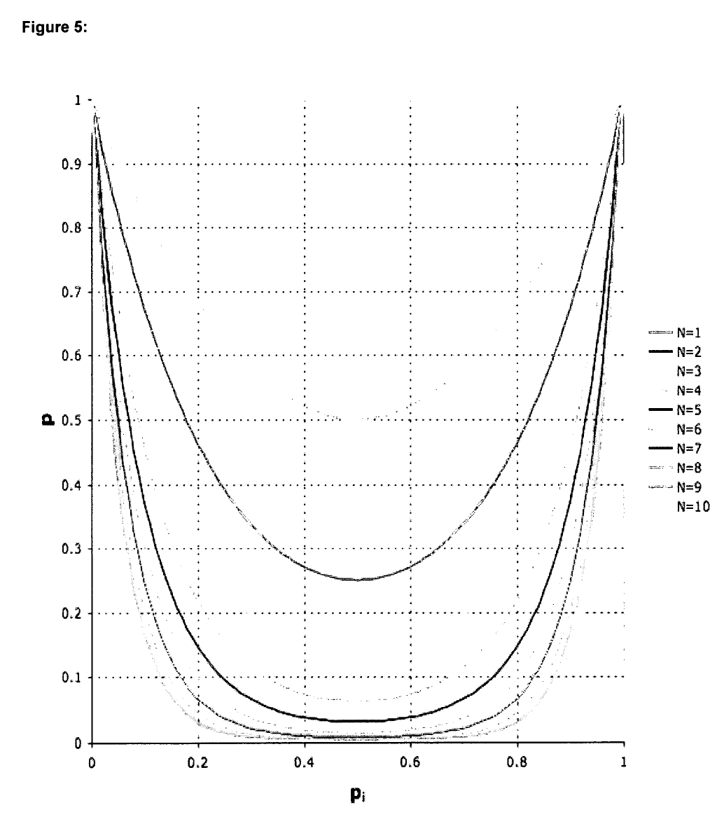

To circumvent this problem, MHC class I and class II alleles with phenotypic frequencies between 0.2 and 0.8 (which corresponds to 20%-80%) on www.allelefrequencies.net are used [45]. Other allele combinations listed in http://www.anthonynolan.orq.uk/HIG/lists/class1list.html or http://www.anthonynolan.org.uk/HIG/lists/class2list.html could also be a candidate. The threshold of 0.2 and 0.8 was calculated from the expected frequency necessary to distinguish individuals with a given probability

$$p = {( {p}_{i}^{2} + {( 1 - {p}_{i} )}^{2} )}^{n} -> N = \frac{ln ( p )}{ln ( {p}_{i}^{2} + {( 1 - {p}_{i} )}^{2} )}$$

where pᵢ is the probability with which a single test field shows a positive result, p the probability that two randomly drawn users have the same MHC profile (see FIG. 5) and N the number of test fields on the assay (9 in our examples, see FIG. 2a). For our examples we use 0.2<pᵢ<0.8. The number of test fields is preferably greater than 5 with a preferred upper limit of 18-33.

It will be apparent to those skilled in the art that various modifications and variations can be made to the objects of the present invention without departing from the spirit or scope of the invention. Thus, it is intended that the present invention cover the modifications and variations of this invention provided they come within the scope of the appended claims and their equivalents. Accordingly, the invention is not limited by the embodiments described above which are presented as examples only but can be modified in various ways within the scope of protection defined by the appended patent claims. All documents cited herein are incorporated by reference as if included in this specification in their entireties.

TABLE 1
Selection of MHC class I alleles with corresponding peptide ligands to obtain
the desired result for individual MHC profiles from the lateral flow assay.
Bold letters within selected ligands mark anchor residues likely to bind
to groves of the corresponding MHC membrane protein.

| | | Average | | | | | | | |
|---|---|---|---|---|---|---|---|---|---|
| | | phenotype | | | | | | | |
| | | frequency | | | | | | | Analytes |
| | | in | | | | | | | used |
| | | Populations | Number of | | | | | | for the |
| | MHC allele | with | populations | Amino acid | | Mass of | | Scores | used for |
| | (WHO assigned | 0.2 < pᵢ | with | sequence of | SEQ | ligand | Examples of | for | examples |
| MHC | type, where | <0.8 | 0.2 < pᵢ | selected | ID | (g/mol) | predicted MHC | predicted | 1 and 2 |
| class | available) | (%)¹ | <0.8 | ligand² | NO: | rounded | epitopes | epotopes³ | (2 × 9) |
| I | HLA-A*01 (A1) | 29.5 | 22 | YTDLLRLFEY | 1 | 1332 | A*01 nonamers | 22, 16 | 1 |
| | HLA-A*02 (A2) | 44.2 | 65 | SLLTSSKGQLQK | 2 | 1288 | A*0201 decamers | 24, 11, 7 | 1 |
| | HLA-A*03 (A3) | 25.2 | 15 | | | | | | |
| | HLA-A*11 (A11) | 40.0 | 17 | | | | | | |
| | HLA-A*24 (A24) | 35.5 | 30 | EYPDRIMNTF | 3 | 1284 | A*0201 nonamers | 6, 5 | 2 |
| | HLA-A*26 (A26) | 32.6 | 3 | ETFYVDGAANR | 4 | 1241 | A*26 nonamers | 24, 5, 3 | 1 |
| | HLA-A*30 (A30) | 27.6 | 7 | | | | | | |
| | HLA-A*31 (A31) | 38.7 | 6 | | | | | | |
| | HLA-A*68 (A68) | 23.6 | 7 | | | | | | |
| | HLA-B*07 (B7) | 27.8 | 16 | VGGLKNWLVHRL | 5 | 1305 | A*0201 decamers | 25, 9, 2 | 2 |
| | HLA-B*08 (B8) | 25.4 | 13 | MPHEKHYTL | 6 | 1234 | B*08 octamers | 18, 10 | 2 |
| | HLA-B*15 (B15) | 29.7 | 28 | | | | | | |
| | HLA-B*35 (B35) | 29.9 | 24 | WASRELERF | 7 | 1192 | B*18 octamers | 13, 3 | |
| | HLA-B*40 (B40) | 43.8 | 23 | REIAQDFKTD | 8 | 1221 | A*03 nonamers | 13, 9 | 1 |
| | HLA-B*44 (B44) | 27.1 | 20 | DEYIYRHFF | 9 | 1288 | B*08 octamers | 17, 6 | |
| | HLA-B*51 (B51) | 25.2 | 12 | DTPLIPLTIF | 10 | 1128 | A*0201 nonamers | 14, 7 | 2 |
| | HLA-Cw*01 (Cw1) | 31.1 | 13 | | | | | | |
| | HLA-Cw*03 (Cw3) | 39.6 | 26 | SAYGEPRKL | 11 | 1019 | B*08 octamers | 12, 6 | 2 |
| | HLA-Cw*04 (Cw4) | 31.8 | 34 | | | | | | |
| | HLA-Cw*07 (Cw7) | 45.1 | 39 | | | | | | |

1) Data from www.allelefrequencies.net
2) Data from www.syfpeithi.de
3) Scores are based on the frequency of the respective amino acids in natural ligands, T-cell epitopes
or binding peptides. For details see Rammensee et al. (1999)

TABLE 2
Selection of MHC class II alleles with corresponding peptide ligands to obtain
the desired result for individual MHC profiles from the lateral flow assay.
Bold letters within selected ligands mark anchor residues likely to bind
to groves of the corresponding MHC membrane protein.

| | | Average | | | | | | | |
|---|---|---|---|---|---|---|---|---|---|
| | | phenotype | | | | | | | |
| | | frequency | | | | | | | |
| | | in | | | | | | | Analytes |
| | | Populations | Number of | | | | Examples | | used |
| | MHC allele | with | populations | Amino | | Mass of | of | | for the |
| | (WHO assigned | 0.2 < pᵢ | with | acid sequence | SEQ | ligand | predicted | Scores for | examples |
| MHC | type, where | <0.8 | 0.2 < pᵢ | of selected | ID | (g/mol) | MHC | predicted | 1 and 2 |
| class | available) | (%)¹ | <0.8 | ligand² | NO: | rounded | epitopes | epitopes³ | (2 × 9) |
| II | HLA-DPB1*0201 | 33.2 | 18 | EIVDLMCHAT | 12 | 1130 | A*0201 | 12, 6 | 1 |
| | | | | | | | nonamers | | |
| | HLA-DPB1*0301 | 30.1 | 10 | | | | | | |
| | HLA-DPB1*0401 | 56 | 19 | IAFNSGMEPGVVAEKV | 13 | 1646 | A*03 | 16, 11, 6, | 2 |
| | | | | | | | decamers | 7, 3, 3 | |
| | HLA-DQA1*0101 | 28.3 | 10 | | | | | | |
| | HLA-DQA1*0102 | 34.7 | 23 | | | | | | |
| | HLA-DQA1*0201 | 26.8 | 13 | QPQQPQQSFPEQERP | 14 | 1822 | B*2705 | 16, 12, 9, | 1 |
| | | | | | | | nonamers | 5, 4, 3, 1 | |
| | HLA-DQA1*03 | 40.5 | 14 | | | | | | |
| | HLA-DQA1*0301 | 47.8 | 6 | HKLQDASAEVERLRR | 15 | 1806 | A*0201 | 16, 16, 12, | 1 |
| | | | | | | | decamers | 7, 6, 3 | |
| | HLA-DQA1*05 | 48.5 | 9 | | | | | | |
| | HLA-DQA1*0501 | 47.1 | 17 | | | | | | |
| | HLA-DQA1*0505 | 41.1 | 5 | | | | | | |
| | HLA-DRB1*01 | 23.3 | 19 | | | | | | |
| | (DR1) | | | | | | | | |
| | HLA-DRB1*03 | 25.8 | 20 | IPENLFLKSDGRVKYT | 16 | 1879 | A*03 | 26, 16, 14, | 2 |
| | (DR3) | | | | | | nonmers | 7, 5, 3, 3 | |
| | HLA-DRB1*0301 | 24.0 | 17 | ISNQLTLDSNTKYFHK | 17 | 2162 | DRB1*0301 | 38, 17, 10, | 2 |
| | (DR17) | | | LN | | | 15 mers | 3 | |
| | HLA-DRB1*04 | 30.0 | 35 | | | | | | |
| | (DR4) | | | | | | | | |
| | HLA-DRB1*0701 | 28.3 | 18 | KVDLTFSKQHALLCSD | 18 | 2660 | DRB1*0701 | 22, 14, 10, | 1 |
| | (DR7) | | | YQADYES | | | 15-mers | 10, 6, 6, 6, | |
| | | | | | | | | 5, 0 | |
| | HLA-DRB1*11 | 30.7 | 32 | KIFYVYMKRKYEAMT | 19 | 1970 | B*1501 | 12, 9, 4, 1, | 1 |
| | (DR11) | | | | | | decamers | 0, −1 | |
| | HLA-DRB1*13 | 26.8 | 27 | | | | | | |
| | (DR13) | | | | | | | | |
| | HLA-DRB1*1501 | 26.6 | 11 | DVGVYRAVTPQGRPDA | 20 | 1699 | DRB1*1501 | 18, 8 | 2 |
| | (DR15) | | | | | | 15-mers | | |

4) Data from www.allelefrequencies.net
5) Data from www.syfpeithi.de
6) Scores are based on the frequency of the respective amino acids in natural ligands, T cell epitopes
or binding peptides. For details see Rammensee et al. (1999)

The following tables 3-22 show various ligands for a number of selected MHC antigens:

TABLE 3
Peptide Ligands of HLA-A*01

| Amino acid sequence of Ligand | SEQ ID NO: |
|---|---|
| NTDSPLRY | 21 |
| PTDPKVVVY | 22 |
| VTEIDQDKY | 23 |
| AIDQLHLEY | 24 |
| SIDRTVMYY | 25 |
| LLDIRSEY | 26 |
| ETDLLDIRSEY | 27 |
| FLDASGAKLDY | 28 |
| FIDASRLVY | 29 |
| TVDQVKDLY | 30 |
| LLDQGQLNKY | 31 |
| YTETEPYHNY | 32 |
| STDKFKTDFY | 33 |
| IADMGHLKY | 34 |
| ATDFKFAMY | 35 |
| YTDYGGLIFNSY | 36 |
| VAVGRALYY | 37 |
| YLDDPDLKY | 38 |
| HLDLGILYY | 39 |
| VTDSIRDEY | 40 |
| LTDRELEEY | 41 |
| YTSDYFISY | 42 |
| ATDYHVRVY | 43 |
| VSDIVGPDGLVY | 44 |
| LEDVVIERY | 45 |
| ASDFITKMDY | 46 |
| YSEEECRQY | 47 |
| STDHIPILY | 48 |
| FTDVNSILRY | 49 |
| HTDDPLTWDY | 50 |
| TTEESLRNYY | 51 |
| STDHIPILY | 52 |
| LSDLGKLSY | 53 |
| QSEDGSHTIQIMY | 54 |
| YIDEQFERY | 55 |
| DSDGSFFLY | 56 |
| DSDGSFFLY | 57 |
| YTAVVPLVY | 58 |
| YTAVVPLVY | 59 |
| LTDRGVMSY | 60 |
| RADGNFLLY | 61 |
| GTDELRLLY | 62 |
| VLDPYLLKY | 63 |
| QVDPLSALKY | 64 |
| YSEKIVDMY | 65 |
| TSDQQRQLY | 66 |
| LAELAHREY | 67 |
| SSEQTFMYY | 68 |
| DTDHYFLRY | 69 |
| ELEDSTLRY | 70 |
| YVDPQFLTY | 71 |
| IADMGHLKY | 72 |
| YTDPEVFKY | 73 |
| STDVSDLLHQY | 74 |
| YTDLLRLFEY | 1 |
| QAQADSLTVY | 75 |
| STDKAEYTFY | 76 |
| MIEPRTLQY | 77 |
| MIEPRTLQY | 78 |
| TTEVHPELY | 79 |
| TSPSQSLFY | 80 |
| TTEQHGARY | 81 |
| HTQGPVDGSLY | 82 |
| STDPSVLGKY | 83 |
| YTDKIDRY | 84 |
| ITDSAGHILY | 85 |
| VTEIFRQAF | 86 |
| HTQGPVDGSLY | 87 |
| QLEDGRTLSDY | 88 |
| IVDCLTEMY | 89 |
| ALRPSTSRSLY | 90 |
| ISNQEPLKL | 91 |
| YSEHPTFTSQY | 92 |
| GSEELRSLY | 93 |
| YAETKHFLY | 94 |
| CTELKLSDY | 95 |
| VSDGGPNLY | 96 |
| VTEPGTAQY | 97 |
| VYDFFVWLHY | 98 |
| EADPTGHSY | 99 |
| EVDPIGHLY | 100 |
| IVDCLTEMY | 101 |
| ILDSSEEDK | 102 |
| KCDICTDEY | 103 |
| SSDYVIPIGTY | 104 |
| DSDPDSFQDY | 105 |

TABLE 4
Peptide Ligands of HLA-A*03

| Amino acid sequence of Ligand | SEQ ID NO: |
|---|---|
| KLFDHAVSKF | 106 |
| SLWDRLIFH | 107 |
| SLLTSSKGQLQK | 108 |
| TSALPIIQK | 109 |
| SLFSRLFGK | 110 |
| KLYEMILKR | 111 |
| GTYAPAEVPK | 112 |
| RLLEMILNK | 113 |
| KLFDKLLEY | 114 |
| RIIEETLALK | 115 |
| IVALILSTK | 116 |
| KIKSFEVVF | 117 |
| KIADRFLLY | 118 |
| GLYEFFRAK | 119 |
| FVQMMTAK | 120 |
| KVMQQNLVY | 121 |
| KVFQEPLFY | 122 |
| GLMGFIVYK | 123 |
| QISIPFLLK | 124 |
| KLADFGLAR | 125 |
| RMFDMGFEY | 126 |
| KVYENYPTY | 127 |
| TVAVPLVGK | 128 |
| ESYEALPQH | 129 |
| ATDPNILGR | 130 |
| TLADLLALR | 131 |
| SLMHSFILK | 132 |
| RVHAYIISY | 133 |
| SVLSPLLNK | 134 |
| KIKSFEVVF | 135 |
| TLADILLYY | 136 |
| RVFSGLVSTGLK | 137 |
| AVAIKAMAK | 138 |
| ILENIQRNK | 139 |
| IVAGSLITK | 140 |
| IIYKGGTSR | 141 |
| QIASFILLR | 142 |
| ATGSWDSFLK | 143 |
| SVYVYKVLK | 144 |
| KVCNPIITKL | 145 |
| KLFIGGLSF | 146 |
| RLFVGSPIK | 147 |
| TLIDIMTRF | 148 |
| NVIRDAVTY | 149 |
| ALLDGSNVVFK | 150 |
| GLFEVGAGWIGK | 151 |
| RVQEAVESMVK | 152 |
| VVNKVPLTGK | 153 |
| ALPSRILLWK | 154 |
| RLLQKVLAY | 155 |
| SLAEILLKK | 156 |
| AVNAHSNILK | 157 |
| ATFPDTLTY | 158 |
| HLEDIVRQK | 159 |
| AVFPKPFVEK | 160 |
| QTYVGITEK | 161 |
| AIIDHIFAS | 162 |
| TVMELVKIIYK | 163 |
| KVYNIQIRY | 164 |
| SVYVLVRQK | 165 |
| FPNSPKWTS | 166 |
| SLFPNSPKWTSK | 167 |
| SIFKQPVTK | 168 |
| VLFGKALNPK | 169 |
| RVFPWFSVK | 170 |
| KTYGEIFEK | 171 |
| GVLAWVKEK | 172 |
| GVADKILKK | 173 |
| ALLAVGATK | 174 |
| KLLNYAPLEK | 175 |
| AMLDTVVFK | 176 |
| GSYNKVFLAK | 177 |
| RILFFNTPK | 178 |
| AALVASGVALY | 179 |
| SIFDGRVVAK | 180 |
| KLPNFGFVVF | 181 |
| GLASFKSFLK | 182 |
| LTGPVMPVR | 183 |
| QLYWSHPRK | 184 |
| LVFPSEIVGK | 185 |
| AVIVLVENFYK | 186 |
| RLYGPSSVSF | 187 |
| VLYDRVLKY | 188 |
| GTLPHPLQR | 189 |
| TSPSQSLFY | 190 |
| YLADFLLTK | 191 |
| RQIPYTMMK | 192 |
| RVYIGRLSY | 193 |
| SVYDSVLQK | 194 |
| GLTDVILYH | 195 |
| SIGERDLIFH | 196 |
| SITSVFITK | 197 |
| GTMTGMLYK | 198 |
| RIHFPLATY | 199 |
| QIFVKTLTGK | 200 |
| SQNFPGSQK | 201 |
| ALRDVRQQY | 202 |
| ALRPSTSRSLY | 203 |
| SLYASSPGGVY | 204 |
| SLYASSPGGVYATR | 205 |
| RTGPPMGSR | 206 |
| HSATGFKQSSK | 207 |
| KQSSKALQR | 208 |
| RLRAEAQVK | 209 |
| NTYASPRKF | 210 |
| RVCEKMALY | 211 |
| QVPLRPMTYK | 212 |
| TVYYGVPVWK | 213 |
| RLRDLLLIVTR | 214 |
| RLRPGGKKK | 215 |
| KIRLRPGGK | 216 |
| ILRGSVAHK | 217 |
| SLFRAVITK | 218 |
| RLEDVFAGK | 219 |
| ASGPGGGAPR | 220 |
| KLIETYFSK | 221 |
| LIYRRRLMK | 222 |
| ALNFPGSQK | 223 |
| QVHPQKVTK | 224 |
| RIETRSARH | 225 |
| LLGPGRPYR | 226 |

TABLE 5
HLA-A*24

| Amino acid sequence of Ligand | SEQ ID NO: |
|---|---|
| RYFDEPVEL | 227 |
| VYISEHEHF | 228 |
| IYTSSVNRL | 229 |
| KYDEIFYNL | 230 |
| KYITQGQLLQF | 231 |
| EYIVKKIPV | 232 |
| LYILSSHDI | 233 |
| AYQHLFYLL | 234 |
| KYFLKPEVL | 235 |
| KYPSPFFVF | 236 |
| RYSSMAASF | 237 |
| KYLSVQGQL | 238 |
| TYGEIFEKF | 239 |
| YYMIGEQKF | 240 |
| YYEEQHPEL | 241 |
| SYIEHIFEI | 242 |
| TYTDRVFFL | 243 |
| VYIKHPVSL | 244 |
| KYPENFFLL | 245 |
| KYPENFFLL | 246 |
| KFIDTTSKF | 247 |
| LYPQFMFHL | 248 |
| RYIPTAAAF | 249 |
| VYPDGIRHI | 250 |
| KYILLMDII | 251 |
| AYVHMVTHF | 252 |
| KFIDPIYQVW | 253 |
| KYQKGFSLW | 254 |
| EYPDRIMNTF | 3 |
| NYIDKVRFL | 255 |
| EYCLKFTKL | 256 |
| SYLDSGIHF | 257 |
| QYSWFVNGTF | 258 |
| RYSIFFDY | 259 |
| TYGPVFMCL | 260 |
| DYCNVLNKEF | 261 |
| RWGLLLALL | 262 |
| RYLKDQQLL | 263 |
| KYKLKHIVW | 264 |
| LFCASDAKAY | 265 |
| QYNKPLCDLL | 266 |
| EYRHYCYSL | 267 |
| AYAQKIFKIL | 268 |
| NYKHCFPEI | 269 |
| IMPKAGLLI | 270 |
| KTPFVSPLL | 271 |
| AYGLDFYIL | 272 |
| LYVDSLFFL | 273 |
| EYRGFTQDF | 274 |
| AFLPWHRLF | 275 |
| AFLPWHRLF | 276 |

TABLE 6
HLA-A*26

| Amino acid sequence of Ligand | SEQ ID NO: |
|---|---|
| EVIPMFSAL | 277 |
| ETFYVDGAANR | 4 |

TABLE 7
HLA-B*07

| Amino acid sequence of Ligand | SEQ ID NO: |
|---|---|
| APAPPPKPM | 278 |
| PSRDSLPLPV | 279 |
| VPNQKRLTLL | 280 |
| IARNLTQQL | 281 |
| RPELVRPAL | 282 |
| KPRSPVVEL | 283 |
| WPLWRLVSL | 284 |
| SPRAAEPVQL | 285 |
| RPKSNIVLL | 286 |
| FVIETARQL | 287 |
| APSPRPLSL | 288 |
| IVPANFPSL | 289 |
| MPRGVVVTL | 290 |
| LPKPPGRGV | 291 |
| APLLRWVL | 292 |
| VPDSSGPERIL | 293 |
| APKRPPSAF | 294 |
| AASKERSGVSL | 295 |
| APRTLVLLL | 296 |
| APRTVLLLL | 297 |
| LVMAPRTVL | 298 |
| APRALLLLL | 299 |
| RVMAPRALL | 300 |
| APRALLLLL | 301 |
| RVMAPRALL | 302 |
| APRTVALTALL | 303 |
| APRTVALTA | 304 |
| APRTVALTAL | 305 |
| VGGLKNWLVHRL | 5 |
| SPHLANYFYF | 306 |
| RPTLWAAAL | 307 |
| SPRLPVGGF | 308 |
| RPSTPKSDSEL | 309 |
| GPPGTGKTDVAVQI | 310 |
| RPAKSMDSL | 311 |
| KPNANRIAL | 312 |
| TPMGPGRTV | 313 |
| RPVSPGKDI | 314 |
| GPRSASLLS | 315 |
| SPSVDKARAEL | 316 |
| RPASAGAML | 317 |
| LVLMVLYLI | 318 |
| APRIITGPAPVL | 319 |
| IPRAALLPLL | 320 |
| KPRDVSSVEL | 321 |
| SPRENILVSL | 322 |
| APARLFALL | 323 |
| SPRYIFTML | 324 |
| RPSGPGPEL | 325 |
| HASDRIIAL | 326 |
| VVYVGGILTIL | 327 |
| VPAPAGPIV | 328 |
| CRVLCCYVL | 329 |
| TPRVTGGGAM | 330 |
| GPRLGVRAT | 331 |
| FPVRPQVPL | 332 |
| TPGPGIRYPL | 333 |
| RPMTYKAAL | 334 |
| RPNNNTRKSI | 335 |
| IPRRIRQGL | 336 |
| GPGHKARVL | 337 |
| SPRTLNAWV | 338 |
| ATPQDLNTM | 339 |
| RVRFFFPSL | 340 |
| MPNDPNRNV | 341 |
| SPSSNRIRNT | 342 |
| NPKASLLSL | 343 |

TABLE 8
HLA-B*08

| Amino acid sequence of Ligand | SEQ ID NO: |
|---|---|
| VPKLKVCAL | 344 |
| YLKVKGNVF | 345 |
| FPRLKSKL | 346 |
| VGLIRNLAL | 347 |
| SLKEKVLL | 348 |
| MPLLRQEEL | 349 |
| LPKVKLAL | 350 |
| ELLIRKLP | 351 |
| ILKQKIADL | 352 |
| FTKVKPLL | 353 |
| YLLEKSRAI | 354 |
| EAFVRHIL | 355 |
| WVKEKVVAL | 356 |
| PASKKTDPQK | 357 |
| DIHHKVLSL | 358 |
| ELKVKNLEL | 359 |
| DAKIRIFDL | 360 |
| EPKYKTQL | 361 |
| MPHEKHYTL | 6 |
| DPMKARVVL | 362 |
| DLKQKNEL | 363 |
| YGMPRQIL | 364 |
| DLERKVESL | 365 |
| HPKYKTEL | 366 |
| GFKQSSKAL | 367 |
| RAKFKQLL | 368 |
| FLRGRAYGL | 369 |
| QAKWRLQTL | 370 |
| AYPLHEQHG | 371 |
| YIKSFVSDA | 372 |
| HSKKKCDEL | 373 |
| WPTVRERM | 374 |
| FLKEKGGL | 375 |
| WPTVRERM | 376 |
| EIKDTKEAL | 377 |
| GGKKKYKL | 378 |
| ELRSLYNTV | 379 |
| EIYKRWIIL | 380 |
| GEIYKRWII | 381 |
| DCKTILKAL | 382 |
| EIYKRWII | 383 |
| YLKDQQLL | 384 |
| GPKVKQWPL | 385 |
| RVKEKYQHL | 386 |
| TLHEYMLDL | 387 |
| ELRRKMMYM | 388 |
| ELRSRYWAI | 389 |
| ADRGLLRDI | 390 |
| RPIIRPATL | 391 |
| LRKPKHKKL | 392 |
| ASKNKEKAL | 393 |
| LPHNHTDL | 394 |

TABLE 9
HLA-B*35

| Amino acid sequence of Ligand | SEQ ID NO: |
|---|---|
| TSEHSHFSL | 395 |
| DDVWTSGSDSDEELV | 396 |
| VFPTKDVAL | 397 |
| VPLRPMTY | 398 |
| TAVPWNASW | 399 |
| WASRELERF | 7 |
| NSSKVSQNY | 400 |
| NPDIVIYQY | 401 |
| PPIPVGDIY | 402 |
| VPVWKEATTTL | 403 |
| VPLDEDFRKY | 404 |
| TVLDVGDAY | 405 |
| HPDIVIYQY | 406 |
| NPVPVGNIY | 407 |
| PPSGKGGNY | 408 |
| MPNDPNRNV | 409 |
| LPIILKALY | 410 |
| --- [skipped] | [411] |

TABLE 10
HLA-B*40

| Amino acid sequence of Ligand | SEQ ID NO: |
|---|---|
| REKIVEVMF | 412 |
| LESLDQLEL | 413 |
| HEETPPTTS | 414 |
| SEDKKNIIL | 415 |
| GEVDVEQHT | 416 |
| KEPEINTTL | 417 |
| HEDKIRVVL | 418 |
| GEGDFLAEGGGV | 419 |
| GEITGEVRM | 420 |
| GEITGEVHM | 421 |
| GELTGEVRM | 422 |
| REIAQDFKTD | 8 |
| TELLIRKL | 423 |
| EEFYVDLER | 424 |
| GEILDVFDA | 425 |
| TEARFGAQL | 426 |
| AEIRHVLVTL | 427 |
| YEELVRMVL | 428 |
| TESGLNVTL | 429 |
| IEVDGKQVEL | 430 |
| GESDDSILRL | 431 |
| SESPIVVVL | 432 |
| TEHGVEVVL | 433 |
| NEFPEPIKL | 434 |
| AEPEEVEVL | 435 |
| KESTLHLVL | 436 |
| KESTLHLVL | 437 |
| REKLQEEML | 438 |
| SEVQDRVML | 439 |
| KEVNSQLSL | 440 |
| RESEEESVSL | 441 |

TABLE 11
HLA-B*44

| Amino acid sequence of Ligand | SEQ ID NO: |
|---|---|
| AEVDKVTGRF | 442 |
| TEKVLAAVY | 443 |
| TERELKVAY | 444 |
| DEVKFLTV | 445 |
| DEVRTLTY | 446 |
| DEAAFLERL | 447 |
| EEIREAFRVF | 448 |
| NEQDLGIQY | 449 |
| DEYIYRHFF | 9 |
| DEVELIHF | 450 |
| NENSLFKSL | 451 |
| DEFKVVVV | 452 |
| LEGLTVVY | 453 |
| TENDIRVMF | 454 |
| KEHVIQNAF | 455 |
| NEFSLKGVDF | 456 |
| EEVKLIKKM | 457 |
| NELPTVAF | 458 |
| VEAIFSKY | 459 |
| SEAGSHTLQW | 460 |
| EEFGQAFSF | 461 |
| VESPLSVSF | 462 |
| VEVLLNYAY | 463 |
| SEVKFTVTF | 464 |
| DERTFHIFY | 465 |
| EEVHDLERKY | 466 |
| DEVKLPAKL | 467 |
| GEAVVNRVF | 468 |
| DEGKVIRF | 469 |
| KENPLQFKF | 470 |
| IETIINTF | 471 |
| DEMKVLVL | 472 |
| EEASLLHQF | 473 |
| DEGRLVLEF | 474 |
| KESTLHLVL | 475 |
| EEIDLRSVGW | 476 |
| NEIEDTFRQF | 477 |
| EEIAFLKKL | 478 |
| DELELHQRF | 479 |
| VEITPYKPTW | 480 |
| KEHVIQNAF | 481 |
| EENLLDFVRF | 482 |
| CEDVPSGKL | 483 |
| HERNGFTVL | 484 |
| QEFFWDANDIY | 485 |
| SEHPTFTSQY | 486 |
| NEGLGWAGW | 487 |
| AENLWVTVY | 488 |
| AENLWVTVYY | 489 |
| FEDLRVLSF | 490 |
| GEISPLPSL | 491 |
| AEMKTDAATL | 492 |
| SELFRSGLDSY | 493 |

TABLE 12
HLA-B*51

| Amino acid sequence of Ligand | SEQ ID NO: |
|---|---|
| LPLKMLNI | 494 |
| YALPLKMLNI | 495 |
| MPFATPMEA | 496 |
| VPYGSFKHV | 497 |
| DTPLIPLTIF | 498 |
| DTPLIPLTIF | 10 |
| NAHEGQLVI | 499 |
| IPLTEEAEL | 500 |
| DPNPQEVVL | 501 |
| RAIEAQQHL | 502 |
| NANPDCKTI | 503 |
| TAFTIPSIY | 504 |

TABLE 13
HLA-Cw*03

| Amino acid sequence of Ligand | SEQ ID NO: |
|---|---|
| SAYGEPRKL | 11 |

TABLE 14
HLA-DPB1*0201

| Amino acid sequence of Ligand | SEQ ID NO: |
|---|---|
| EIVDLMCHAT | 12 |

TABLE 15
HLA-DPB1*0401

| Amino acid sequence of Ligand | SEQ ID NO: |
|---|---|
| TQHFVQENYLEY | 505 |
| IAFNSGMEPGVVAEKV | 13 |

TABLE 16
HLA-DQA1*0201

| Amino acid sequence of Ligand | SEQ ID NO: |
|---|---|
| FYLLYYTEFTPTEKD | 509 |
| QPQQPQQSFPEQERP | 14 |
| LPSTEDVYDCRVE | 510 |
| TEDVYDCRVEHWGLD | 511 |
| EPRAPWIEQEGPEYWD | 512 |
| LPKPPKPVSKMRMATPLLMQ | 513 |
| LRSLDRNLPSDSQDLGQHGLE | 514 |

TABLE 17
HLA-DQA1*0301/DQB1*0301 (DQ3.1)

| Amino acid sequence of Ligand | SEQ ID NO: |
|---|---|
| LNFDFQAKQLSDP | 506 |
| HKLQDASAEVERLRR | 15 |
| STFDAGAGIALNDH | 507 |
| TAADTAAQITQR | 508 |

TABLE 18
HLA-DRB1*03

| Amino acid sequence of Ligand | SEQ ID NO: |
|---|---|
| IPENLFLKSDGRVKYT | 515 |
| EPLPLKVNEQRKAV | 516 |
| QPPKLLIYWASTRE | 517 |
| LKIDLANRETSI | 518 |
| PTLVPIEYKNMI | 519 |
| RHMGSDWSIEIDGKKYTAQE | 520 |
| DSDKNPLFLDEQLIRAEFQR | 521 |
| QPSVQIQVYQGEREIASHNK | 522 |
| IQMISEAARFQYIEG | 523 |
| WEKMKASEKIFYVYM | 524 |

TABLE 19
HLA-DRB1*11

| Amino acid sequence of Ligand | SEQ ID NO: |
|---|---|
| PTFTSQYRIQGKLE | 525 |
| IQMISEAARFQYIEG | 526 |
| KIFYVYMKRKYEAMT | 527 |
| IVHSATGFKQSSKALQRPVASDFEP | 528 |
| NSNHVASGAGEAAIETQSSSSEEIV | 529 |

TABLE 20
HLA-DRB1*0301

| Amino acid sequence of Ligand | SEQ ID NO: |
|---|---|
| VFLLLLADKVPETSLS | 530 |
| VDTFLEDVKNLYHSEA | 531 |
| VTTLNSDLKYNALDLTN | 532 |
| ISNQLTLDSNTKYFHKLN | 17 |
| ISNQLTLDSNTKYFHKL | 533 |
| ISNQLTLDSNTKYFHK | 534 |
| YANILLDRRVPQTDMTF | 535 |
| NLFLKSDGRIKYTLNKNSLK | 536 |
| IPDNLFLKSDGRIKYTLNKN | 537 |
| IPDNLFLKSDGRIKYTLNK | 538 |
| IPDNLFLKSDGRIKYTLN | 539 |
| IPDNLFLKSDGRIKYTL | 540 |
| NLFLKSDGRIKYTLNK | 541 |
| NLFLKSDGRIKYTLN | 542 |
| GKFAIRPDKKSNPIIRTV | 543 |
| VGSDWRFLRGYHQYA | 544 |
| VDDTQFVRFDSDAASQ | 545 |
| LNKILLDEQAQWK | 546 |
| GPPKLDIRKEEKQIMIDIFH | 547 |
| GPPKLDIRKEEKQIMIDIFHP | 548 |
| KQTISPDYRNMI | 549 |
| LPKPPKPVSKMRMATPL | 550 |
| LPKPPKPVSKMRMATPLLMQALP | 551 |
| LPKPPKPVSKMRMATPLLMQALP | 552 |
| PKPPKPVSKMRMATPL | 553 |
| PKPPKPVSKMRMATPLLMQA | 554 |
| KPPKPVSKMRMATPLLMQ | 555 |
| KPPKPVSKMRMATPLLMQALPM | 556 |
| ATKYGNMTEDHVMHLLQNA | 557 |
| KPRAIVVDPVHGFMY | 558 |
| LLSFVRDLNQYRADI | 559 |
| GDVVAVDIKEKGKDKWIELK | 560 |
| MGRSIKVQLQ | 561 |
| SDKNPLFLDEQLI | 562 |
| KTIAYDEEARR | 563 |
| YLQVPSPSMGRDIKVQFQ | 564 |
| TIRLTAADHRQLQLS | 565 |
| WEKMKASEKIFYVYM | 566 |

TABLE 21
HLA-DRB1*0701

| Amino acid sequence of Ligand | SEQ ID NO: |
|---|---|
| VTQYLNATGNRWCSWSLSQAR | 567 |
| VTQYLNATGNRWCSWSL | 568 |
| KVDLTFSKQHALLCSDYQADYES | 18 |
| KVDLTFSKQHALLCS | 569 |
| FSHDYRGSTSHRL | 570 |
| LPKYFEKKRNTII | 571 |
| APVLISQKLSPIYNLVPVK | 572 |
| VPGLYSPCRAFFNKEELL | 573 |
| VPGLYSPCRAFFNKEB | 574 |
| VGSDWRFLRGYHQYAYDG | 575 |
| RPAGDGTFQKWASVVVPSGQ | 576 |
| RPAGDGTFQKWASVVV | 577 |
| GDGTFQKWASVVVPS | 578 |
| GTFQKWASVVVPSGHL | 579 |
| GTFQKWASVVVPSGQ | 580 |
| GTFQKWASVVVPSGQEQRYTCHV | 581 |
| RETQISKTNTQTYRE | 582 |
| RETQISKTNTQTYRE | 583 |
| RETQISKTNTQTYRE | 584 |
| SLQSPITVEWRAQSESAQSKMLSGIGGFVL | 585 |
| RSNYTPITNPPEVTVLTNSPVELREP | 586 |
| GALANIAVDKANLEIMTKRSN | 587 |
| APSPLPETTENVVCALGLTV | 588 |
| TPSYVAFTDTERLIGDA | 589 |
| TPSYVAFTDTERLIG | 590 |
| KHKVYACEVTHQGL | 591 |
| PKPPKPVSKMRMATPLLMQALP | 592 |
| GDMYPKTWSGMLVGALCALAGVLTI | 593 |
| TSILCYRKREWIK | 594 |
| PAFRFTREAAQDCEV | 595 |
| SAYLAHRNQSLDLAEQELVDCAS | 596 |
| QIYPPNANKIREALAQTHSAIAHWT | 597 |
| PGPLRESIVCYFMVFLQTHI | 598 |
| KALENKKKQLGAGGK | 599 |
| FRKQNPDIVIQYMDDLYVG | 600 |
| AKIELSSSQSTSVNLPYITV | 601 |
| RPGLLGASVLGDDIhT | 602 |
| PKYVKQNTLKLATGMRNVP | 603 |
| RMPTVLQCVNVSVVS | 604 |
| AEGLRALLARSHVER | 605 |
| TIRLTAADHRQLQLS | 606 |
| EYLNKIQNSLSTEWSPCSVT | 607 |
| DALESIMTTKSVSFR | 608 |
| ALTGGMELTRDPTVP | 609 |
| KIFYVYMKRKYEAMT | 610 |

TABLE 22
HLA-DRB1*1501

| Amino acid sequence of Ligand | SEQ ID NO: |
|---|---|
| EAEQLRAYLDGTGVE | 611 |
| DVGVYRAVTPQGRPDA | 20 |
| LEEFGRFASFEAQG | 612 |
| YAYVAREQSCR | 613 |
| AGLTLSLLVICSYLFISRG | 614 |
| VIGLYGNGV | 615 |
| MSIYVYALPLKMLNI | 616 |
| GRHLIFCHSKRKCDELATKL | 617 |
| AHYNIVTFCCKCD | 618 |
| RVVINKDTTIII | 619 |
| PVVHFFKNIVT | 620 |
| SKYLATASTMDHARHGTLPR | 621 |
| AEGLRALLARSHVER | 622 |
| WTTCQSIAFPSKTSASIGSL | 623 |
| LFCGCGHEALTGTEKLIETY | 624 |
| RHRPLQEVYPEANAPIGHNRE | 625 |

### Example 3

### Customization of Odours

For this example, the decrypted MHC profile, obtained with the rapid assay as described above may correspond to positive results for the three following detected MHC peptides of said user with the sequences: EYPDRIMNTF (SEQ ID NO:3), DEYIYRHFE (SEQ ID NO:626) and IAFNSGMEPGWAEKV (SEQ ID NO:13). Alternatively the latter three peptides could be the peptides best matching the MHC profile of said user. For the discussion below it is always assumed that detected MHC peptides of a given user reflect MHC class I or II alleles as has been described in example 2 and the tables 1 and 2 therein. Therefore, MHC profile and peptides detected in a sample of a given user are interchangeable and customization of odours could in principle be based on a genetic MHC profile of a given user (through sequencing, genetic marker analysis such as short tandem repeats etc.).

It has to be understood that although this example is limited to three peptides it can be any number of peptides from tables 1 and 2 (depending on the number of peptides for which a detection zone has been provided with the rapid assay, in the example depicted in FIG. 2 this would be 9 peptides). In principle, however, the list of possible peptides is not limited to those mentioned in tables 1 and 2, it can contain any known MHC peptide detected as analyte in a sample of a given user or any MHC peptide matching to detected analytes in a sample of a given user.

Now imagine said user wants to have a fragrance customized to match its own MHC profile for “enhance” or “complement”. The following gives an example of how this works:

The peptides mentioned above, which can either be the peptides detected in a sample of said user or the peptides matching to the peptides detected in a sample of said user, have been synthesised with standard procedures (e.g. NMI Peptides, Reutlingen Germany). For this example, the Characteristics of synthetic peptides for the customization of odours, amino acid sequence (AS seq.), molecular weight (MW), mass, HPLC-purity, odour note as rated by participants and overall charge are given in the table below:

TABLE 23

| | | | | HPLC | Odour Note | Consistency | |
|---|---|---|---|---|---|---|---|
| No. | AS seq. | MW | Mass | purity | (N = 20) | (N = 20) | Charge |
| 1 | H-EYPDR IMNTF-OH | 1285.44 | 1284.58 | 77.8 | Acidic, Rubber | 0.8 | −1 |
| | (SEQ ID NO: 3) | | | | | | |
| 2 | H-DEYIY RHFE-OH | 1289.41 | 1288.59 | 78.3 | Mild, Acidic | 0.6 | 0 |
| | (SEQ ID NO: 626) | | | | | | |
| 3 | H-IAFNS GMEPG VVAEK V-OH | 1647 | 1646.83 | 88.2 | Mossy | 1 | −2 |
| | (SEQ ID NO: 13) | | | | | | |

The amount of 2.125 mg of each of the three peptides was dissolved in 6 ml of deionised water. Twenty participants have classified the resulting test solution for odour note. In a blind test, odours were presented in a standardized way on 150 mm Ø filter paper (Whatman). The average consistency with which the individual solutions could be recognized has been recorded. These solutions can be used as additive for any existing fragrance product, they can be used to produce a mixture of the synthetic peptides, which can again be applied directly to surfaces of any kind or be added to existing fragrances. Dissolved peptides provide stock solutions for any application for which the surrounding odour environment is intended to be customized to reflect “enhance” and “complement” of a given user or even a group of users.

To explain in more detail what is meant with “enhance” and “complement” we refer to the distance metrics defined in the detailed description of the invention and in example 1. Therein, the MHC profile Rᵢ of a given user i comprises of a set of Boolean {r_{i,1}, r_{i,2}, . . . r_{i,k} . . . r_{i,N}} which contains the information on the detected analytes, e.g. MHC peptides {P₁, P₂, . . . Pₖ, . . . P_{N}}, by the rapid assay. (e.g. N=the number of analytes tested=9 detection zones, FIG. 2).

The general instruction to create a customized odour for “enhance”, where Ψᵢ is the set of indices k of the result r_{i,k}, with a positive result, i.e. r_{i,k}=1, of user i and Pₖ is the MHC peptide corresponding to the detected analyte k is:

Ψᵢ={k|∀r_{i,k}=1}

enhanceᵢ={Pₖ|∀kεΨᵢ}

and the general instruction to customize an odour for “complement”, where Ωᵢ is the set of MHC profiles Rⱼ complementary to the MHC Rᵢ profile of user i with a given range for the quality of match Δd (e.g. Δd=0, optimal match, Δd>0, optimal match and matches within given range), is:

$${Ω}_{i} = \{ {R}_{j} \vert ( ∀ j \neq i ) ⋀ ( \frac{N}{2} + Δ d < {d}_{i , j} \leq \frac{N}{2} + Δ d ) \}$$

Iᵢ is the set of indices j of the complementary MHC profiles Rⱼ out of the set Ωᵢ.

Iᵢ={j|∀RⱼεΩᵢ}

and Ψⱼ is the set of indices k of the result r_{j,k} of the complementary MHC profiles Rⱼ with a positive result, i.e. r_{j,k}=1.

Ψⱼ{k|∀r_{j,k}=1}

the customized odour for the complementary MHC profile Rⱼ is contains the set of analytes Pₖ with the index k of the set Ψⱼ.

complement_{i,j}={Pₖ|∀kεΨⱼ}

The set of customized odours (otherᵢ) complementary to the MHC profile Rᵢ is contains all customized odours (other_{i,j}) where j is element of the set Iᵢ.

complementᵢ={other_{i,j}|∀jεIᵢ}

### REFERENCES

1. Wedekind, C. and D. Penn, MHC genes, body odours, and odour preferences. Nephrology Dialysis Transplantation, 2000. 15: p. 1269-1271.
2. Eggert, F., et al., The major histocompatibility complex and the chemosensory signalling of individuality in humans. Genetica, 1999. 104: p. 265-273.
3. Penn, D. J. and W. K. Potts, The evolution of mating preferences and major histocompatibility complex. The American Naturalist, 1999. 153(2): p. 145-164.
4. Wedekind, C., et al., MHC-dependent mate preferences in humans. Proc. R. Soc. Lond. B, 1995. 260: p. 245-249.
5. Singh, P. B., Chemosensation and genetic individuality. Reproduction, 2001. 121: p. 529-539.
6. Penn, D. J., et al., Individual and gender fingerprints in human body odour. J. R. Soc. Interface, 2006. 4: p. 331-340.
7. Restrepo, D., et al., Odortypes and MHC peptides: complementary chemosignals of MHC haplotype. Trends in Neurosciences, 2006. 29(11).
8. Milinski, M., et al., Mate choice decisions of stickleback females predictably modified by MHC peptide ligands. Prodeedings of the National Academy of Science, 2005. 102(12): p. 4414-4418.
9. Ables, E. M., L. M. Kay, and J. M. Mateo, Rats assess degree of relatedness from human odors. Physiology & Behavior, 2007. 90: p. 726-732.
10. Wyart, C., et al., Smelling a single component of male sweat alters levels of cortisol in women. Journal of Neuroscience, 2007. 27(6): p. 1261-1265.
11. Milinski, M. and C. Wedekind, Evidence for MHC-correlated perfume preferences in humans. Behavioral Ecology, 2001. 12(2): p. 140-149.
12. Jacob, S., et al., Paternally inherited HLA alleles are associated with women's choice of male odor. Nature Genetics, 2002. 30(2): p. 175-179.
13. Garver-Apgar, C. E., et al., Major histocompatibility complex alleles, sexual responsivity, and unfaithfulness in romantic couples. PsychSc, 2006. 17(10): p. 830-836.
14. Milinski, M., The major histocompatibility complex, sexual selection, and mate choice. Annual Review in Ecology, Evolution, and Systematics, 2006. 37: p. 159-186.
15. Nowak, M. A., K. Tarczy-Hornoch, and J. M. Austy, Proc. Natl. Acad. Sci., 1992. 89: p. 10896.
16. DeBoer, R. J. and A. S. Perelson, Proceedings of the Royal Society London B, 1993. 252: p. 171.
17. Reusch, T. B. H., et al., Female sticklebacks count alleles in a strategy of sexual selection explaining MHC polymorphism. Nature, 2001. 414: p. 300-302.
18. Wegner, K. M., et al., Parasite selection for immunogenetic optimality. Science, 2003. 301: p. 1343.
19. Zerefos, P. G., et al., Characterization of the human urine proteome by preparative electrophoresis in combination with 2-DE. Proteomics, 2006. 6: p. 4346-4355.
20. Singer, A. G., G. K. Beauchamp, and K. Yamazaki, Volatile signals of the major histocompatibility complex in male mouse urine. Proc. Natl. Acad. Sci., 1997. 94: p. 2210-2214.
21. Krieger, J., et al., Selective activation of G protein subtypes in the vomeronasal organ upon stimulation with urine-derived compounds. The Journal of Biological Chemistry, 1999. 274(8): p. 4655-4662.
22. Leinders-Zufall, T., et al., MHC class I peptides as chemosensory signals in the vomeronasal organ. Science, 2004. 306(5): p. 1033-1037.
23. Montag, S., et al., “Electronic nose” detects major histocompatibility complex-dependent prerenal and postrenal odor components. Proc. Natl. Acad. Sci., 2001. 98: p. 9249-9254.
24. Boehm, T. and F. Zufall, MHC peptides and the sensory evaluation of genotype. Trends in Neurosciences, 2006. 29(2).
25. Rammensee, H.-G., et al., SYFPEITHI: database for MHC ligands and peptide motifs. Immunogenetics, 1999. 50: p. 213-219.
26. Yewdell, J. W. and J. R. Bennink, Immunodominance in major histocompatibility complex class I-restircet T lymphocyte responses. Annual Review in Immunology, 1999. 17: p. 1-30.
27. Zavazava, N., G. Leimenstoll, and W. Müller-Ruchholtz, Measurement of soluble MHC class I molecules in renal graft patients: a noninvasive allograft monitor. Journal of Clinical Laboratory Analysis, 1990. 4: p. 426-429.
28. Adamashvili, I., et al., Soluble HLA: patterns of expression in normal subjects, autoimmune diseases, and transplant recipients. Rheumatol Int, 2005. 25: p. 491-500.
29. Wobst, B., et al., Molecular forms of soluble HLA in body fluids: potential determinants of body odor cues. Genetica, 1999. 104: p. 275-283.
30. Adamashvili, I., et al., Soluble HLA in saliva of patients with autoimmune rheumatic diseases. Rheumatol Int, 2002. 22: p. 71-76.
31. Hayman, M. W., et al., Soluble human leukodyte antigen: A diagnostic indicator of rheumatoid arthritis. Journal of Immunological Methods, 2006. 315: p. 19-26.
32. Amirghofran, Z., et al., Journal of Cancer Research Clinical Oncology. 128, 2002(443-448).
33. Utsumi, M., et al., Expression of major urinary protein genes in the nasal glands associated with general olfaction. Neurobiology, 1999. 39: p. 227-236.
34. Brennan, P. A. and F. Zufall, Pheromonal communication in vertebrates. Nature, 2006. 444: p. 308
35. Fan, J., et al., Visual perception of male body attractiveness. Proceedings of the Royal Society London B, 2005. 272: p. 219-226.
36. Tovée, M. J., et al., Effects of sexual dimorphism on facial attractiveness. Lancet, 1999. 399: p. 215-216.
37. Perrett, D. I., et al., Effects of sexual dimorphism on facial attractiveness. Nature, 1998. 394: p. 884-887.
38. Buss, D. M., The evolution of desire: strategies of human mating. 1994, New York: Basic Books.
39. Gourab Goshal, P. H., Attractiveness and activity in internet comunities. Physica, 2006. 364: p. 603-609.
40. Schreuder, G. M. T., et al., The HLA Dictionary 2004: a summary of HLA-A, -B, -C,-DRB1/3/4/5 and-DQB1 alleles and their association with serologically defined HLA-A, -B, -C, -DR and-DQ antigens. Tissue Antigens, 2005. 65: p. 1-55.
41. Singh, P. B., R. E. Brown, and B. Roser, MHC antigens in urine as olfactory recognition cues. Nature, 1987. 327: p. 161-164.
42. Reynolds, J. B., B. S. Weir, and C. C. Cockerham, Estimation of the coancestry coefficient: basis for a short-term genetic distance. Genetics, 1983. 105: p. 767-779.
43. Cavalli-Sforza, L. L. and A. W. F. Edwards, Phyligenetic analysis: models and estimation procedures. Evolution, 1967. 32: p. 550-570.
44. Nei, M., Genetic distance between populations. American Naturalist, 1972. 106: p. 283-292.
45. Midleton, D., et al., www.allelefrequencies.net New allele frequency database. Tissue Antigens, 2003. 61: p. 403-407.

1. A method for the preparation of one or more customized odour compositions, said method comprising the following steps:
(a) providing an MHC profile of one or more individuals, wherein said MHC profile contains information on the presence or absence of N different MHC antigens in each individual, wherein N is an integer greater than 3;
(b) for each individual, selecting at least one peptide ligand of an MHC antigen found to be expressed by said individual and/or selecting at least one peptide ligand of an MHC antigen found not to be expressed by said individual;
(c) for each individual, providing at least one compound comprising said at least one peptide ligand selected in step (b), or a fragment or derivative thereof;
(d) for each individual, mixing said at least one compound with a cosmetically acceptable excipient, diluent and/or vehicle to obtain said customized odour composition.

2. The method according to claim 1, wherein N is an integer from 8 to 15.

3. The method according to claim 1, wherein said N different MHC antigens are selected from the group consisting of A*01; A*02; A*0201; A*03; A*11; A*1101; A*24; A*2402; B*07; B*0702; B*08; B*0801; B*13; B*1301; B*15; B*1525; B*35; B*39; B*3901; B*40; B*4001; B*4002; B*44; B*48; B*4801; Cw*01; Cw*0102; Cw*03; Cw*0304; Cw*030401; Cw*04; Cw*0401; Cw*0403; Cw*0501; Cw*06; Cw*0602; Cw*07; Cw*0701; Cw*0702; Cw*08; Cw*0801; DPB1*0201; DPB1*0301; DPB1*0401; DPB1*0402; DPB1*0501; DQA1*0101; DQA1*0102; DQA1*0103; DQA1*0201; DQA1*03; DQA1*0301; DQA1*0401; DQA1*05; DQA1*0501; DQB1*02; DQB1*0201; DQB1*0202; DQB1*0301; DQB1*0302; DQB1*0402; DQB1*0501; DQB1*06; DQB1*0601; DQB1*0602; DRB1*01; DRB1*03; DRB1*0301; DRB1*04; DRB1*07; DRB1*0701; DRB1*08; DRB1*0802; DRB1*080302; DRB1*09; DRB1*090102; DRB1*11; DRB1*110101; DRB1*12; DRB1*120201; DRB1*13; DRB1*14; DRB1*1401; DRB1*15; DRB1*1501; DRB1*150101; and DRB1*160201.

4. The method according to claim 3, wherein said N different MHC antigens are selected from the group consisting of A*01, A*02, A*0201, A*11, A*1101, A*24, A*2402, B*1301, B*3901, B*4001, Cw*03, Cw*030401, Cw*07, Cw*0701, Cw*0702, DPB1*0201, DPB1*0401, DPB1*0402, DQA1*0102, DQA1*03, DQA1*0301, DQA1*05, DQA1*0501, DQB1*02, DQB1*0201, DQB1*0301, DQB1*06, DRB1*04, DRB1*080302, and DRB1*120201.

5. The method according to claim 1, wherein step (b) further comprises selecting peptide ligands of all MHC antigens found to be expressed in said individual, and not selecting any peptide ligand of MHC antigens found not to be expressed in said individual.

6. The method according to claim 1, wherein step (b) further comprises selecting peptide ligands of all MHC antigens found not to be expressed in said individual, and not selecting any peptide ligand of MHC antigens found to be expressed in said individual.

7. The method according to claim 1, wherein step (b) further comprises selecting peptide ligands of 25 to 75% of all MHC antigens found to be expressed in said individual.

8. The method according to claim 7, wherein step (b) further comprises selecting 25 to 75% of all MHC antigens found not to be expressed in said individual.

9. The method according to claim 1, wherein step (a) further comprises determining a plurality of MHC profiles of a plurality of individuals.

10. The method according to claim 1, wherein step (c) further comprises synthesizing said compound.

11. The method according to claim 1, wherein the MHC profile is determined using a test kit for the rapid determination of an individual's MHC profile, said test kit comprising:
at least one solid substrate in contact with a proximal sample application zone, said substrate having N conjugation zones and N distal detection zones, said conjugation zones respectively containing N labelled primary binding reagents capable of binding with an analyte to form an analyte-primary binding reagent conjugate; said distal detection zone respectively having immobilized thereunto, N unlabeled secondary binding reagents capable of binding to a migrating analyte-labeled primary binding reagent conjugate to determine the presence or absence of N analytes which are indicative of defined N-MHC antigens, wherein N is an integer greater than 3, preferably from 6 to 30.

12. An odour composition obtained by the method according to claim 1, wherein said composition comprises at least one peptide.

13. The customized odour composition according to claim 12, wherein said composition comprises at least two different peptides.

14. The customized odour composition according to claim 13, wherein said composition comprises at least three different peptides.

15. The customized odour composition according to claim 12, wherein the concentration of said at least one peptide in the composition ranges from 0.01 to 0.8 gL⁻¹.

16. The customized odour composition according to claim 12, wherein the length of said at least one peptide ranges from 7 to 18 amino acids.

17. The customized odour composition according to claim 12, wherein said peptide(s) comprised in said composition is a/are ligand(s) of an MHC antigen selected from the group consisting of A*01; A*02; A*0201; A*03; A*11; A*1101; A*24; A*2402; B*07; B*0702; B*08; B*0801; B*13; B*1301; B*15; B*1525; B*35; B*39; B*3901; B*40; B*4001; B*4002; B*44; B*48; B*4801; Cw*01; Cw*0102; Cw*03; Cw*0304; Cw*030401; Cw*04; Cw*0401; Cw*0403; Cw*0501; Cw*06; Cw*0602; Cw*07; Cw*0701; Cw*0702; Cw*08; Cw*0801; DPB1*0201; DPB1*0301; DPB1*0401; DPB1*0402; DPB1*0501; DQA1*0101; DQA1*0102; DQA1*0103; DQA1*0201; DQA1*03; DQA1*0301; DQA1*0401; DQA1*05; DQA1*0501; DQB1*02; DQB1*0201; DQB1*0202; DQB1*0301; DQB1*0302; DQB1*0402; DQB1*0501; DQB1*06; DQB1*0601; DQB1*0602; DRB1*01; DRB1*03; DRB1*0301; DRB1*04; DRB1*07; DRB1*0701; DRB1*08; DRB1*0802; DRB1*080302; DRB1*09; DRB1*090102; DRB1*11; DRB1*110101; DRB1*12; DRB1*120201; DRB1*13; DRB1*14; DRB1*1401; DRB1*15; DRB1*1501; DRB1*150101; and DRB1*160201.

18. The customized odour composition according to claim 17, wherein said peptide(s) comprised in said composition is a/are ligand(s) of an MHC antigen selected from the group consisting of A*01, A*02, A*0201, A*11, A*1101, A*24, A*2402, B*1301, B*3901, B*4001, Cw*03, Cw*030401, Cw*07, Cw*0701, Cw*0702, DPB1*0201, DPB1*0401, DPB1*0402, DQA1*0102, DQA1*03, DQA1*0301, DQA1*05, DQA1*0501, DQB1*02, DQB1*0201, DQB1*0301, DQB1*06, DRB1*04, DRB1*080302, and DRB1*120201.

19. A method for enhancing an individual's own body odour, said method comprising the step of administering or applying to the individual the customized odour composition according to claim 12.

20. (canceled)

21. A method for the manufacture of a perfume composition, said method comprising the step of adding a fragrance to the customized odour composition according to claim 12.

22. (canceled)
